(19) United States
(12) Patent Application Publication
(10) Pub. No.: US 2010/0266574 A1
(43) Pub. Date: Oct. 21, 2010

(54) Oligoribonucleotides and Methods of Use Thereof for Treatment of Fibrotic Conditions and Other Diseases

(76) Inventors: Orna Mor, Kiryat Ono (IL); Elena Feinstein, Rehovot (IL)

Correspondence Address: Cooper & Dunham, 1185 Avenue of the Americas, New York, NY, 10036, US

(21) Appl. No.: 11/921,985

(22) PCT Filed: Jun. 8, 2006
(86) PCT No.: PCT/IL06/00671

§ 371 Date: Jan. 22, 2008

Related U.S. Application Data
(60) Provisional application No. 60/689,616, filed on Jun. 10, 2005.

(51) Int. Cl. A61K 39/395; C07H 21/02; C12N 15/63; A61K 31/712; A61P 1/16; A61P 27/06

(52) U.S. Cl. 424/130.1; 536/24.5; 435/320.1; 514/44 A

(57) ABSTRACT

The invention relates to a double-stranded compound, preferably an oligoribonucleotide, which down-regulates the expression of a gene of the TGase family at post-transcriptional level. The invention also relates to a pharmaceutical composition comprising the compound, or a vector capable of expressing the oligoribonucleotide, and a pharmaceutically acceptable carrier. The present invention also contemplates a method of treating a patient suffering from fibrotic disease such as kidney and liver fibrosis and ocular scarring comprising administering to the patient the pharmaceutical composition in a therapeutically effective amount so as to thereby treat the patient. The invention also relates to treatment of fibrotic and other diseases by use of antibodies to TGase polypeptides.

This application claims priority of U.S. provisional patent application Ser. No. 60/689616, filed Jun. 10, 2005, which is hereby incorporated by reference. Throughout this application various patent and scientific publications are cited. The disclosures for these publications in their entireties are hereby incorporated by reference into this application to more fully describe the state of the art to which this invention pertains. In particular co-assigned patent application PCT/IL 2005/000102 filed 27 Jan. 2005 is hereby incorporated by reference into this application.

### BACKGROUND OF THE INVENTION

siRNAs and RNA Interference

RNA interference (RNAi) is a phenomenon involving double-stranded (ds) RNA-dependent gene specific posttranscriptional silencing. Originally, attempts to study this phenomenon and to manipulate mammalian cells experimentally were frustrated by an active, non-specific antiviral defense mechanism which was activated in response to long dsRNA molecules; see Gil et al. 2000, Apoptosis, 5:107-114. Later it was discovered that synthetic duplexes of 21 nucleotide RNAs could mediate gene specific RNAi in mammalian cells, without the stimulation of the generic antiviral defence mechanisms see Elbashir et al. Nature 2001, 411:494-498 and Caplen et al. Proc Natl Acad Sci 2001, 98:9742-9747. As a result, small interfering RNAs (siRNAs), which are short double-stranded RNAs, have become powerful tools in attempting to understand gene function.

Thus, RNA interference (RNAi) refers to the process of sequence-specific post-transcriptional gene silencing in mammals mediated by small interfering RNAs (siRNAs) (Fire et al, 1998, Nature 391, 806) or microRNAs (miRNAs) (Ambros V. Nature 431:7006,350-355(2004); and Bartel D P. Cell. 2004 Jan 23; 116(2): 281-97 MicroRNAs: genomics, biogenesis, mechanism, and function). The corresponding process in plants is commonly referred to as specific post-transcriptional gene silencing or RNA silencing and is also referred to as quelling in fungi. An siRNA is a double-stranded RNA molecule which down-regulates or silences (prevents) the expression of a gene/mRNA of its endogenous (cellular) counterpart. RNA interference is based on the ability of dsRNA species to enter a specific protein complex, where it is then targeted to the complementary cellular RNA and specifically degrades it. Thus, the RNA interference response features an endonuclease complex containing an siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir et al 2001, Genes Dev., 15, 188). In more detail, longer dsRNAs are digested into short (17-29 bp) dsRNA fragments (also referred to as short inhibitory RNAs—“siRNAs”) by type III RNAses (DICER, DROSHA, etc., Bernstein et al., Nature, 2001, v. 409, p. 363-6; Lee et al., Nature, 2003, 425, p. 415-9). The RISC protein complex recognizes these fragments and complementary mRNA. The whole process is culminated by endonuclease cleavage of target mRNA (McManus & Sharp, Nature Rev Genet, 2002, v. 3, p. 737-47; Paddison & Hannon, Curr Opin Mol Ther. 2003 June; 5(3): 217-24). For information on these terms and proposed mechanisms, see Bernstein E., Denli A M. Hannon G J: 2001 The rest is silence. RNA. I; 7(11): 1509-21; Nishikura K.: 2001 A short primer on RNAi: RNA-directed RNA polymerase acts as a key catalyst. Cell. I 16; 107(4): 415-8 and PCT publication WO 01/36646 (Glover et al).

The selection and synthesis of siRNA corresponding to known genes has been widely reported; see for example Chalk A M, Wahlestedt C, Sonnhammer E L. 2004 Improved and automated prediction of effective siRNA Biochem. Biophys. Res. Commun. June 18; 319(1): 264-74; Sioud M, Leirdal M., 2004, Potential design rules and enzymatic synthesis of siRNAs, Methods Mol Biol.; 252:457-69; Levenkova N, Gu Q, Rux J J. 2004, Gene specific siRNA selector Bioinformatics. I 12; 20(3): 430-2. and Ui-Tei K, Naito Y, Takahashi F, Haraguchi T, Ohki-Hamazaki H, Juni A; Ueda R; Saigo K., Guidelines for the selection of highly effective siRNA sequences for mammalian and chick RNA interference Nucleic Acids Res. 2004 I 9;32(3):936-48.Se also Liu Y, Braasch D A, Nulf C J, Corey D R. Efficient and isoform-selective inhibition of cellular gene expression by peptide nucleic acids, Biochemistry, 2004 I 24;43 (7):1921-7. See also PCT publications WO 2004/015107 (Atugen) and WO 02/44321 (Tuschl et al), and also Chiu Y L, Rana T M. siRNA function in RNAi: a chemical modification analysis, RNA 2003 September;9(9):1034-48 and I U.S. Pat. Nos. 5,898,031 and 6,107,094 (Crooke) for production of modified/more stable siRNAs.

Several groups have described the development of DNA-based vectors capable of generating siRNA within cells. The method generally involves transcription of short hairpin RNAs that are efficiently processed to form siRNAs within cells. Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553. These reports describe methods to generate siRNAs capable of specifically targeting numerous endogenously and exogenously expressed genes.

siRNA has recently been successfully used for inhibition in primates; for further details see Tolentino et al., Retina 24(1) February 2004 I 132-138.

Transglutaminase (TGase) Family

The TGase polypeptides (EC 2.3.2.13) are a family of proteins that act as TGase enzymes with cross-linking activities (i.e. they catalyse reactions resulting in protein cross-links and/or covalent incorporation of biogenic amines). TGase polypeptides further catalyse the formation of a covalent glutamyl-lysyl bond, a unique isopeptide bond that is highly resistant to proteolysis and denaturants and that cannot be disrupted by any known vertebrate endopeptidase.

The family comprises 9 different enzymes among which are the factor XIIIa (plasma transglutaminase), keratinocyte transglutaminase (TGaseI also termed TGase 1), epidermal transglutaminase (TGaseIII also termed TGase 3), prostate transglutaminase (TGaseIV also termed TGase 4), Transglutaminase 5 (TGx also termed TGase 5), Transglutaminase 7 (TGz also termed TGase 7) and tissue-type transglutaminase (TGase II). Although the overall primary structure of these enzymes is different, they all share a common amino acid sequence at the active site (Y-G-Q-C-W) and a strict calcium dependence for their activity (Lesort M, Tucholski J, Miller M L, Johnson G V, Tissue transglutaminase: a possible role in neurodegenerative diseases. Prog Neurobiol. 2000 August; 61(5):439-63).

Abberant activity of the enzymes of the TGase family is characteristics of several neurodegenerative diseases, such as Alzheimer disease (AD), Parkinson disease (PD), supranuclear palsy and Huntington disease (HD), is associated with celiac disease, (Transglutaminases—possible drug targets in human diseases, impaired wound healing, autoimmunity, diabetes, articular cartilage calcification, atherosclerosis, cancer metastasis, skin disorders and fibrotic diseases. (Fesus L, Piacentini M, Transglutaminase 2: an enigmatic enzyme with diverse functions. Trends Biochem Sci. 2002 October;27(10):534-9; Karpouzas G A, Terkeltaub R A, New developments in the pathogenesis of articular cartilage calcification. Curr Rheumatol Rep. 1999 December;1(2):121-7; Aeschlimann D, Thomazy V., Protein crosslinking in assembly and remodelling of extracellular matrices: the role of transglutaminases. Connect Tissue Res. 2000;41(1):1-27.; Ishida-Yamamoto A, Iizuka H., Structural organization of comified cell envelopes and alterations in inherited skin disorders. Exp Dermatol. 1998 February;7(1): 1-10)

Transglutaminase I

Transglutaminase type 1 (TGaI) is a member of the TGase class of enzymes that catalyze the cross-linking of proteins, a characteristic feature of epidermal differentiation and squamous metaplasia. TGM1(transglutaminase 1) crosslinks the cornified envelope of mature keratinocytes. TGase I is a Ca(2+)-dependent enzyme which catalyzes epsilon-(gamma-glutamyl)lysine cross-linking of substrate proteins. In the skin such proteins are involucrin and loricrin to generate the cornified envelope at the cell periphery of the stratum corneum(Inada et al Facilitated wound healing by activation of the Transglutaminase 1 gene. Am J Pathol. 2000 December;157(6):1875-82.). Appropriate expression of the TGM1 gene is crucial for proper keratinocyte function as inactivating mutations lead to the debilitating skin disease, lamellar ichthyosis. TGM1 is also expressed in squamous metaplasia, a consequence in some epithelia of vitamin A deficiency or toxic insult that can lead to neoplasia During epithelial cell differentiation transglutaminase 1 is known to cross-link the cornified envelope proteins involucrin and loricrin. TGase I was shown also to be expressed in normal lung and its expression is evident a normal feature of bronchial epithelium and is linked to the process of squamous differentiation occurring in preinvasive lesions. Its activity was also related to pathological keratinization of ocular surface epithelium (Pathological keratinization of ocular surface epithelium. Adv Exp Med Biol. 2002;506(Pt A):641-617; Nakamura T, Nishida K, Dota A, Matsuki M, Yamanishi K, Kinoshita S. Elevated expression of transglutaminase 1 and keratinization-related proteins in conjunctiva in severe ocular surface disease. Invest Ophthalmol Vis Sci. 2001 March;42(3):549-56).

TGase 3

Epidermal-type transglutaminase (TGase 3) cross-links a variety of structural proteins during the formation of the cornified cell envelope in the epidermis. It is called “epidermal” or “hair follicle” Tgase and is a zymogen, requiring proteolytic activation to achieve maximal specific activity. TGase 3 mRNA is also expressed in the brain, stomach, spleen, small intestine, testis, skeletal muscle and skin. The stomach and testis expressed TGase 3 protein in size similar to that observed in the epidermis. In celiac disease epidermal transglutaminase, rather than tissue transglutaminase, is the dominant autoantigen in dermatitis herpetiformis thus explaining why skin symptoms appear in a proportion of patients having gluten sensitive disease (Epidermal transglutaminase (TGase 3) is the autoantigen of dermatitis herpetiformis. Sardy M, Karpati S, Merkl B, Paulsson M, Smyth N. J Exp Med. 2002 March 18;195(6):747-57). TGase 3 was shown to be expressed in upper layers of epidermis. TGase 3 displayed a diffuse cytoplasmic distribution in vitro consistent with its proposed role in the early phase of cornified cell envelope assembly in the cytoplasm (J Dermatol Sci. 2003 August;32(2):95-103. Analysis of epidermal-type transglutaminase (transglutaminase 3) in human stratified epithelia and cultured keratinocytes using monoclonal antibodies. Hitomi K, Presland R B, Nakayama T, Fleckman P, Dale B A, Maki M.)

TGase 5

Transglutaminase5 was originally cloned from keratinocytes, and a partial biochemical characterisation showed its involvement in skin differentiation, in parallel to TGase1 and TGase 3. It was shown to be able to induce cell death when intracellularly overexpressed and to contain GTP binding domains which are similar to those in transglutaminase 2 (Overexpressed transglutaminase 5 triggers cell death. Cadot B, Rufini A, Pietroni V, Ramadan S, Guerrieri P, Melino G, Candi E. Amino Acids. 2004 July;26(4):405-8). Moreover, it was shown that GTP and ATP inhibit TGase 5 cross-linking activity in vitro, and Ca2+ is capable of completely reversing this inhibition. In addition, TGase 5 mRNA is present in different adult and foetal tissues, suggesting a role for TGase 5 outside the epidermis (Biochem J. 2004 July 1;381(Pt 1):313-9 Transglutaminase 5 is regulated by guanine-adenine nucleotides. Candi E, Paradisi A, Terrinoni A, Pietroni V, Oddi S, Cadot B, Jogini V, Meiyappan M, Clardy J, Finazzi-Agro A, Melino G).

Fibrotic Diseases

Fibrotic diseases are all characterized by the excess deposition of a fibrous material within the extracellular matrix, which contributes to abnormal changes in tissue architecture and interferes with normal organ function. Unfortunately, although fibrosis is widely prevalent, debilitating and often life threatening, there is no effective treatment currently available.

All tissues damaged by trauma respond by the initiation of a wound-healing program. Fibrosis, a type of disorder characterized by excessive scarring, occurs when the normal self-limiting process of wound healing response is disturbed, and causes excessive production and deposition of collagen. As a result, normal organ tissue is replaced with scar tissue, which eventually leads to the functional failure of the organ.

Fibrosis may be initiated by diverse causes and in various organs. Liver cirrhosis, pulmonary fibrosis, sarcoidosis, keloids and kidney fibrosis are all chronic conditions associated with progressive fibrosis, thereby causing a continuous loss of normal tissue function.

Acute fibrosis (usually with a sudden and severe onset and of short duration) occurs as a common response to various forms of trauma including accidental injuries (particularly injuries to the spine and central nervous system), infections, surgery, ischemic illness (e.g. cardiac scarring following heart attack), burns, environmental pollutants, alcohol and other types of toxins, acute respiratory distress syndrome, radiation and chemotherapy treatments).

Ocular Surgery and Ocular Scarring

Contracture of scar tissue resulting from eye surgery may often occur. Glaucoma surgery to create new drainage channels often fails due to scarring and contraction of tissues and the generated drainage system may be blocked requiring additional surgical intervention. Current anti-scarring regimens (Mitomycin C or 5FU) are limited due to the complications involved (e.g. blindness) e.g. see Cordeiro M F, Gay J A, Khaw P T., Human anti-transforming growth factor-beta2 antibody: a new glaucoma anti-scarring agent Invest Ophthalmol Vis Sci. 1999 September;40(10):2225-34. Also there may be contraction of scar tissue formed after corneal trauma or corneal surgery, for example laser or surgical treatment for myopia or refractive error in which contraction of tissues may lead to inaccurate results. Scar tissue may be formed on/in the vitreous humor or the retina, for example, and may eventually causes blindness in some diabetics, and may be formed after detachment surgery, called proliferative vitreoretinopathy (PVR). PVR is the most common complication following retinal detachment and is associated with a retinal hole or break. PVR refers to the growth of cellular membranes within the vitreous cavity and on the front and back surfaces of the retina containing retinal pigment epithelial (RPE) cells. These membranes, which are essentially scar tissues, exert traction on the retina and may result in recurrences of retinal detachment, even after an initially successful retinal detachment procedure.

Scar tissue may be formed in the orbit or on eye and eyelid muscles after squint, orbital or eyelid surgery, or thyroid eye disease, and where scarring of the conjunctiva occurs as may happen after glaucoma surgery or in cicatricial disease, inflammatory disease, for example, pemphigoid, or infective disease, for example, trachoma. A further eye problem associated with the contraction of collagen-comprising tissues is the opacification and contracture of the lens capsule after cataract extraction. Ocular diseases include wound healing, cataract, dry eye, sterile corneal ulceration, recurrent epithelial erosion, corneal neovascularization, pterygium, conjuctivochalasis, glaucoma, PVR, and ocular fibrosis.

Cataract

A cataract is a clouding of the lens in the eye that affects vision. Most cataracts are related to aging. By age 80, more than half of all Americans either have a cataract or have had cataract surgery. A cataract can occur in either or both eyes.

Age-related cataracts develop in two ways:

1. Protein aggregates reduce the sharpness of the image reaching the retina.

2. The clear lens slowly changes to a yellowish/brownish color, adding a brownish tint to vision.

Although most cataracts are related to aging, there are other types of cataract:
Secondary cataract. Cataracts can form after surgery for other eye conditions, such as glaucoma. Cataracts also can develop in people who have other health problems, such as diabetes. Cataracts are sometimes linked to steroid use.
Traumatic cataract. Cataracts can develop after an eye injury, sometimes years later.
Congenital cataract. Some babies are born with cataracts or develop them in childhood, often in both eyes. These cataracts may be so small that they do not affect vision. If they do, the lenses may need to be removed.
Radiation cataract. Cataracts can develop after exposure to certain types of radiation.

Liver Fibrosis

Liver fibrosis (LF) is a generally irreversible consequence of hepatic damage of several etiologies. In the Western world, the main etiologic categories are: alcoholic liver disease (30-50%), viral hepatitis (30%), biliary disease (5-10%), primary hemochromatosis (5%), and drug-related and cryptogenic cirrhosis of, unknown etiology, (10-15%). Wilson's disease, α₁-antitrypsin deficiency and other rare diseases also have liver fibrosis as one of the symptoms. Liver cirrhosis, the end stage of liver fibrosis, frequently requires liver transplantation and is among the top ten causes of death in the Western world.

Kidney Fibrosis and Related Conditions

Chronic Renal Failure (CRF)

Chronic renal failure is a gradual and progressive loss of the ability of the kidneys to excrete wastes, concentrate urine, and conserve electrolytes. CRF is slowly progressive. It most often results from any disease that causes gradual loss of kidney function, and fibrosis is the main pathology that produces CRF.

Diabetic Nephropathy

Diabetic nephropathy, hallmarks of which are glomerulosclerosis and tubulointerstitial fibrosis, is the single most prevalent cause of end-stage renal disease in the modern world, and diabetic patients constitute the largest population on dialysis. Such therapy is costly and far from optimal. Transplantation offers a better outcome but suffers from a severe shortage of donors. More targeted therapies against diabetic nephropathy (as well as against other types of kidney pathologies) are not developed, since molecular mechanisms underlying these pathologies are largely unknown. Identification of an essential functional target gene that is modulated in the disease and affects the severity of the outcome of diabetes nephropathy has a high diagnostic as well as therapeutic value.

Origins of Kidney Pathology

Many pathological processes in the kidney (e.g., glomerular nephritis, physical obstructions, toxic injuries, metabolic and immunological diseases) eventually culminate in similar or identical morphological changes, namely glomerulosclerosis and tubulointerstitial fibrosis. Thus, different types of insults converge on the same single genetic program resulting in two hallmarks of fibrosis: the proliferation of fibroblasts and overproduction by them of various protein components of connective tissue. In addition, thickening of the basal membrane in the glomeruli accompanies interstitial fibrosis and culminates in glomerulosclerosis.

Pulmonary Fibrosis

Interstitial pulmonary fibrosis (IPF) is scarring of the lung caused by a variety of inhaled agents including mineral particles, organic dusts, and oxidant gases, or by unknown reasons (idiopathic lung fibrosis). The disease afflicts millions of individuals worldwide, and there are no effective therapeutic approaches. A major reason for the lack of useful treatments is that few of the molecular mechanisms of disease have been defined sufficiently to design appropriate targets for therapy (Lasky J A., Brody A R. (2000), “Interstitial fibrosis and growth factors”, Environ Health Perspect.;108 Suppl 4:751-62).

Cardiac Fibrosis

Heart failure is unique among the major cardiovascular disorders in that it alone is increasing in prevalence while there has been a striking decrease in other conditions. Some of this can be attributed to the aging of the populations of the United States and Europe. The ability to salvage patients with myocardial damage is also a major factor, as these patients may develop progression of left ventricular dysfunction due to deleterious remodelling of the heart.

The normal myocardium is composed of a variety of cells, cardiac myocytes and noncardiomyocytes, which include endothelial and vascular smooth muscle cells and fibroblasts.

Structural remodeling of the ventricular wall is a key determinant of clinical outcome in heart disease. Such remodeling involves the production and destruction of extracellular matrix proteins, cell proliferation and migration, and apoptotic and necrotic cell death. Cardiac fibroblasts are crucially involved in these processes, producing growth factors and cytokines that act as autocrine and paracrine factors, as well as extracellular matrix proteins and proteinases. Recent studies have shown that the interactions between cardiac fibroblasts and cardiomyocytes are essential for the progression of cardiac remodeling of which the net effect is deterioration in cardiac function and the onset of heart failure (Manabe I, Shindo T, Nagai R. (2002), “Gene expression in fibroblasts and fibrosis: involvement in cardiac hypertrophy”, Circ Res. 13;91(12):1103-13).

Burns and Scars

A particular problem which may arise, particularly in fibrotic disease, is contraction of tissues, for example contraction of scars. Contraction of tissues comprising extracellular matrix components, especially of collagen-comprising tissues, may occur in connection with many different pathological conditions and with surgical or cosmetic procedures. Contracture, for example, of scars, may cause physical problems, which may lead to the need for medical treatment, or it may cause problems of a purely cosmetic nature. Collagen is the major component of scar and other contracted tissue and as such is the most important structural component to consider. Nevertheless, scar and other contracted tissue also comprises other structural components, especially other extracellular matrix components, for example, elastin, which may also contribute to contraction of the tissue. Contraction of collagen-comprising tissue, which may also comprise other extracellular matrix components, frequently occurs in the healing of burns. The burns may be chemical, thermal or radiation burns and may be of the eye, the surface of the skin or the skin and the underlying tissues. It may also be the case that there are burns on internal tissues, for example, caused by radiation treatment. Contraction of burnt tissues is often a problem and may lead to physical and/or cosmetic problems, for example, loss of movement and/or disfigurement.

Skin grafts may be applied for a variety of reasons and may often undergo contraction after application. As with the healing of burnt tissues the contraction may lead to both physical and cosmetic problems. It is a particularly serious problem where many skin grafts are needed as, for example, in a serious burns case.

Contraction is also a problem in production of artificial skin. To make a true artificial skin it is necessary to have an epidermis made of epithelial cells (keratinocytes) and a dermis made of collagen populated with fibroblasts. It is important to have both types of cells because they signal and stimulate each other using growth factors. The collagen component of the artificial skin often contracts to less than one tenth of its original area when populated by fibroblasts.

Cicatricial contraction, contraction due to shrinkage of the fibrous tissue of a scar, is common. In some cases the scar may become a vicious cicatrix, a scar in which the contraction causes serious deformity. A patient's stomach may be effectively separated into two separate chambers in an hour-glass contracture by the contraction of scar tissue formed when a stomach ulcer heals. Obstruction of passages and ducts, cicatricial stenosis, may occur due to the contraction of scar tissue. Contraction of blood vessels may be due to primary obstruction or surgical trauma, for example, after surgery or angioplasty. Stenosis of other hollow visci, for examples, ureters, may also occur. Problems may occur where any form of scarring takes place, whether resulting from accidental wounds or from surgery. Conditions of the skin and tendons which involve contraction of collagen-comprising tissues include post-trauma conditions resulting from surgery or accidents, for example, hand or foot tendon injuries, post-graft conditions and pathological conditions, such as scleroderma, Dupuytren's contracture and epidermolysis bullosa. Scarring and contraction of tissues in the eye may occur in various conditions, for example, the sequelae of retinal detachment or diabetic eye disease (as mentioned above). Contraction of the sockets found in the skull for the eyeballs and associated structures, including extra-ocular muscles and eyelids, may occur if there is trauma or inflammatory damage. The tissues contract within the sockets causing a variety of problems including double vision and an unsightly appearance.

The mechanism and control of contraction of tissues comprising extracellular matrix components, for example, collagen-comprising tissues, is still poorly understood. Some degree of contraction appears to be part of the healing process, but the trigger for contraction is not known.

For further information on different types of fibrosis see: Molina V, Blank M, Shoenfeld Y. (2002), “Fibrotic diseases”, Harefuah, 141(11): 973-8, 1009; Yu L, Noble N A, Border W A (2002), “Therapeutic strategies to halt renal fibrosis”, Curr Opin Pharmacol. 2(2):177-81; Keane W F, Lyle P A. (2003), “Recent advances in management of type 2 diabetes and nephropathy: lessons from the RENAAL study”, Am J Kidney Dis. 41(3 Suppl 2): S22-5; Bohle A, Kressel G, Muller C A, Muller G A. (1989), “The pathogenesis of chronic renal failure”, Pathol Res Pract. 185(4):421-40; Kikkawa R, Togawa M; Isono M; Isshiki K, Haneda M. (1997), “Mechanism of the progression of diabetic nephropathy to renal failure”, Kidney Int Suppl. 62:S39-40; Bataller R, Brenner D A. (2001), “Hepatic stellate cells as a target for the treatment of liver fibrosis”, Semin Liver Dis. 21(3):437-51; Gross T J, Hunninghake G W, (2001) “Idiopathic pulmonary fibrosis”, N Engl J Med. 345(7):517-25; Frohlich E D. (2001) “Fibrosis and ischemia: the real risks in hypertensive heart disease”, Am J Hypertens;14(6 Pt 2):194S-199S; Friedman S L. (2003), “Liver fibrosis—from bench to bedside”, J Hepatol. 38 Suppl 1:S38-53; Albanis E, Safadi R, Friedman S L. (2003), “Treatment of hepatic fibrosis: almost there”, Curr Gastroenterol Rep. 5(1):48-56; (Weber K T. (2000), “Fibrosis and hypertensive heart disease”, Curr Opin Cardiol. 15(4):264-72).

Osteoarthritis

Among the main characteristics of osteoarthritis are the degradation of articular cartilage and the formation of new bone at the joint edges, so-called osteophytes. See Van den Berg W B., Growth factors in experimental osteoarthritis: transforming growth factor beta pathogenic? J Rheumatol Suppl. 1995 February;43:143-5; Scharstuhl A, Glansbeek H L, Van Beuningen H M, Vitters E L, Van der Kraan P M, Van den Berg W B., Inhibition of endogenous TGF-beta during experimental osteoarthritis prevents osteophyte formation and impairs cartilage repair. J Immunol. 2002 July 1;169(1):507-14; Karpouzas G A, Terkeltaub R A., New developments in the pathogenesis of articular cartilage calcification. Curr Rheumatol Rep. 1999 December;1(2):121-7.

Neurological Diseases

Polyglutamine diseases are a group of neurological diseases that are caused by expansion of CAG trinucleotide repeats coding for polyglutamine insert. Polyglutamine diseases include Huntington's disease (HD), spinobulbar muscular atrophy, dentatorubral-pallidoluysian atrophy and spinocerebellar ataxias (SCAs) 1, 2, 3, 6, 7 and 17. All these diseases are characterized by the presence of expansion of polyglutamine stretches (exceeding 35-40 glutamines), thus forming intranuclear aggregates, which leads to neuronal death. Alzheimer's disease (AD) is the most common cause of cognitive impairment in older patients and is expected to increase greatly in prevalence. Neurofibrillary degeneration, associated with the formation of paired helical filaments (PHF), is one of the critical neuropathological hallmarks of Alzheimer's disease (AD). Parkinson disease is a neurodegenerative disorder of aging characterized by a selective and progressive loss of dopaminergic neurons within the substantia nigra. See also Mastroberardino P G, Iannicola C, Nardacci R, Bernassola F, De Laurenzi V, Melino G, Moreno S, Pavone F, Oliverio S, Fesus L, Piacentini M. Tissue transglutaminase ablation reduces neuronal death and prolongs survival in a mouse model of Huntington's disease. Cell Death Differ. 2002 September;9(9):873-80; Karpuj M V, Becher M W, Springer J E, Chabas D, Youssef S, Pedotti R, Mitchell D, Steinman L., Prolonged survival and decreased abnormal movements in transgenic model of Huntington disease, with administration of the transglutaminase inhibitor cystamine. Nat Med. 2002 February;8(2):143-9; Citron B A, Suo Z, SantaCruz K, Davies P J, Qin F, Festoff B W., Protein crosslinking, tissue transglutaminase, alternative splicing and neurodegeneration. Neurochem Int. 2002 January;40(1):69-78; Chen J S, Mehta K., Tissue transglutaminase: an enzyme with a split personality. Int J Biochem Cell Biol. 1999 August;31(8):817-36.

Although the above discussion of diseases and disorders relates in particular to humans, animals and in particular mammals may exhibit the conditions described above or similar or analogous conditions

In conclusion, there are no effective modes of therapy for the prevention and/or treatment of fibrosis in general and for its related pathologies and certainly no effective treatment for contraction of tissues, nor is there effective treatment for ocular scarring. Treatments that are available suffer from, inter alia, the drawbacks of severe side effects due to the lack of selective targeting and there is a need therefore to develop novel compounds and methods of treatment for these purposes.

### SUMMARY OF THE INVENTION

The invention provides novel double stranded oligonlonucleotides. These oligoribonucleotides inhibit the TGase family of genes, in particular one or more of TGase 1, TGase 3, TGase 5 and TGase 7 by the mechanism of RNA interference. The invention also provides a pharmaceutical composition comprising such oligoribonucleotides, and vectors capable of expressing the ribonucleotides.

The present invention also provides a method of treating a patient suffering from a fibrosis-related pathology comprising administering to the patient the oligoribonucleotide typically as a pharmaceutical composition, in a therapeutically effective amount so as to thereby treat the patient. The present invention also contemplates treating other diseases and conditions. The invention also relates to treatment of fibrotic and other diseases by use of an antibody to a TGase polypeptide in particular to one or more of TGase 1, TGase 3, TGase 5 and TGase 7 polypeptide.

### BRIEF DESCRIPTION OF THE FIGURES

FIG. 1. This figure sets forth the nucleotide sequence of the human TGase I gene—SEQ ID NO:1.

FIG. 2 This figure sets forth the amino acid sequence of the human TGase I corresponding polypeptide—SEQ ID NO:2.

### DETAILED DESCRIPTION OF THE INVENTION

The present invention relates generally to compounds which down-regulate i.e. inhibit expression of the TGase gene family particularly to novel small interfering RNAs (siRNAs), and to the use of these novel siRNAs in the treatment of various diseases and medical conditions in particular fibrotic diseases. Moreover, the present invention relates generally to compounds which inhibit expression of one or more of the TGase gene family particularly the TGase I gene, TGase 3 gene, TGase 5 gene and/or TGase 7gene and particularly to novel small interfering RNAs (siRNAs), and to the use of these novel siRNAs in combination with anti TGase gene family siRNAs in the treatment of various diseases and medical conditions in particular fibrotic diseases, cataract and glaucoma. The fibrotic diseases are in particular kidney fibrosis, liver fibrosis, and ocular scarring, cataract, glaucoma and other diseases related to aberrant expression of any of the genes of the Transglutaminase gene family.

Thus, the inhibitor of TGaseII expression (transcription or translation) or polypeptide activity may be inter alia siRNA, antibodies, preferably neutralizing antibodies or fragments thereof, including single chain antibodies, antisense oligonucleotides, antisense DNA or RNA molecules, proteins, polypeptides and peptides including peptido-mimetics and dominant negatives, and also expression vectors expressing all the above. Additional inhibitors may be small chemical molecules, which generally have a molecular weight of less than 2000 daltons, more preferably less than 1000 daltons, even more preferably less than 500 daltons. These inhibitors may act as follows: small molecules may affect expression and/or activity; antibodies may affect activity; all kinds of antisense may affect TGaseII expression; and dominant negative polypeptides and peptidomimetics may affect activity; expression vectors may be used inter alia for delivery of antisense or dominant-negative polypeptides or antibodies.

The present invention provides methods and compositions for inhibiting expression of one or more of the target genes of the TGase gene family in particular one or more of TGase 1, TGase 3, TGase 5 and TGase 7 genes in vivo. In general, the method includes administering oligoribonucleotides, such as small interfering RNAs (i.e., siRNAs) that are targeted to a particular mRNA and hybridize to, or interact with, it under biological conditions (within the cell), or a nucleic acid material that can produce siRNA in a cell, in an amount sufficient to inhibit expression of a target gene by an RNA interference mechanism. In particular, the subject method can be used to inhibit expression of one or more of the TGase1, TGase 3, TGase 5 and TGase 7genes for treatment of disease. Additionally the siRNAs of the invention can be used in vitro as part of a compound screening system to look for small compounds that compete with, or overcome effect of, siRNAs.

In accordance with the present invention, the siRNA molecules or inhibitors such as antibodies of the Transglutaminase(“TGase”)family of genes, in particular at least one of TGase 1, TGase 3, TGase 5 and TGase 7 genes may be used as drugs to treat various pathologies including fibrosis related pathologies (as defined below) and also to treat ocular diseases including cataract, glaucoma, cardiovascular diseases, neurological diseases, polyglutamine diseases (including Huntington's disease (HD), spinobulbar muscular atrophy, dentatorubral-pallidoluysian atrophy and spinocerebellar ataxias (SCAs) 1, 2, 3, 6, 7 and 17), Alzheimer's and Parkinson's disease. and osteoarthritis.

As used herein, the term “TGase 1 gene” is defined as any homolog including any allelic variant thereof of TGase 1 gene having preferably 90% homology, more preferably 95% homology, and even more preferably 98% homology to the amino acid encoding region of SEQ ID NO:1, or nucleic acid sequences which bind to the gene under conditions of highly stringent hybridization, which are well-known in the art (for example, see Ausubel et al., Current Protocols in Molecular Biology, John Wiley and Sons, Baltimore, Md. (1988), updated in 1995 and 1998).

Similarly, as used herein, the term “TGase 3 gene”, or “TGase 5 gene” or “TGase 7gene”, is defined as any homolog including any allelic variant thereof of the TGase 3 gene or TGase 5 gene or TGase 7gene respectively having preferably 90% homology, more preferably 95% homology, and even more preferably 98% homology to the amino acid encoding region of the TGase 3 gene or TGase 5 gene or TGase 7gene respectively, or nucleic acid sequences which bind to the TGase 3 gene or TGase 5 gene or TGase 7gene respectively under conditions of highly stringent hybridization, which are well-known in the art (for example, see Ausubel et al., Current Protocols in Molecular Biology, John Wiley and Sons, Baltimore, Md. (1988), updated in 1995 and 1998). The Genebank references for the genes, which set forth the amino acid encoding region for each gene, are as follows: TGase 3—GI:39777600; TGase 5-GI:4759229 and 42518071 (TGase 5 has 2 splice variants); and TGase 7-GI:16445034.

As used herein, the term “Transglutaminase I polypeptide”, or “TGase I polypeptide”, or is defined as any homolog of TGase I polypeptide having preferably 90% homology, more preferably 95% homology, and even more preferably 98% homology to SEQ ID NO:2, as either full-length or fragments or a domain thereof, as a mutant of the polypeptide encoded by a spliced variant nucleic acid sequence, as a chimera with other polypeptides, provided that any of the above has the same or substantially the same biological function as the TGase I polypeptide. TGase I polypeptide, or a TGase I polypeptide homolog, may be present in different forms, including but not limited to soluble protein, membrane-bound (either in purified membrane preparations or on a cell surface), bead-bound, or any other form presenting TGase I polypeptide or fragments and polypeptides derived thereof.

Similarly, as used herein, the term “TGase 3 polypeptide”, or “TGase 5 polypeptide” or “TGase 7 polypeptide”, is defined as any homolog of TGase 3 polypeptide or TGase 5 polypeptide or TGase 7polypeptide respectively having preferably 90% homology, more preferably 95% homology, and even more preferably 98% homology to the amino acid sequence of TGase 3 polypeptide or TGase 5 polypeptide or TGase 7polypeptide respectively, as either full-length or fragments or a domain thereof, as a mutant of the polypeptide encoded by a spliced variant nucleic acid sequence, as a chimera with other polypeptides, provided that any of the above has the same or substantially the same biological function as the TGase 3, 5, or 7 polypeptide respectively. TGase 3, 5, or 7 polypeptide respectively, or a TGase 3, 5, or 7 polypeptide homolog, may be present in different forms, including but not limited to soluble protein, membrane-bound (either in purified membrane) preparations or on a cell surface), bead-bound, or any other form presenting TGase 3, 5, or 7 polypeptide or fragments and polypeptides derived thereof. The Genebank references for the polypeptides, which set forth the amino acid sequence, are given above.

As used herein, an “interactor” is a molecule with which a TGase polypeptide binds or interacts or activates in nature; for example, a molecule on the surface of a cell that expresses a TGase polypeptide, a molecule on the surface of a second cell or a cytoplasmic molecule. An interactor may be a ligand that is activated by TGase alone or by TGase as a part of a complex with other components. An interactor may be a component of a signal transduction pathway that facilitates transduction of an extracellular signal from TGase through the cell membrane and into the cell. An interactor, for example, can be a second intercellular protein that mediates downstream signaling from TGase. The interactor is a molecule with which TGase binds in competition with a known TGase substrate (e.g. fibronectin).

As used herein, the term “lysyl donor” or “K donor” is defined as any polypeptide having the ability to donate a lysyl side chain to allow the formation of gamma-glutamyl-lysine bonds during transglutamination process.

As used herein, the term “glutamyl donor” or “Q donor” is defined as any polypeptide having the ability to donate glutamine side chain to allow the formation of gamma-glutamyl-lysine bonds during transglutamination process.

The present invention provides double-stranded oligoribonucleotides (siRNAs), which down-regulate (inhibit) the expression of any one of the TGase gene family. The present invention in particular provides double-stranded oligoribonucleotides (siRNAs), which down-regulate the expression of genes TGase I, TGase 3, TGase 5 and TGase 7. The downregulation of the expression of each transglutminase can be measured by e.g., measuring the amount of the lysyl-glutamyl crosslinked material produced in the presence of the siRNAs or by direct assessment of the amounts of TGase mRNA or polypeptide. The amount of TGase mRNA may be measured by e.g., by Northern blotting, RNase protection, RT-PCR or real-time PCR. The amount of TGase polypeptide may be measured by immunoblotting or by immunoprecipitation or by ELISA with TGase-specific antibodies.

An siRNA of the invention is a duplex oligoribonucleotide in which the sense strand is derived from the mRNA sequence of a TGase gene, and the antisense strand is complementary to the sense strand. In general, some deviation from the target mRNA sequence is tolerated without compromising the siRNA activity (see e.g. Czauderna et al 2003 Nucleic Acids Research 31(11), 2705-2716). An siRNA of the invention inhibits gene expression on a post-transcriptional level with or without destroying the mRNA. Without being bound by theory, siRNA may target the mRNA for specific cleavage and degradation and/or may inhibit translation from the targeted message.

More particularly, the invention provides a compound having the structure:
5′(N)ₓ-Z3′ antisense strand
3′Z′-(N′)_{y}5′ sense strand
wherein each N and N′ is a ribonucleotide which may be modified or unmodified in its sugar residue and (N)ₓ and (N′)_{y} is oligomer in which each consecutive N or N′ is joined to the next N or N′ by covalent bond;
wherein each of x and y is an integer between 17 and 40;
wherein each of Z and Z′ may be present or absent, but if present is dTdT and is covalently attached at the 3′ terminus;
and wherein the sequence of (N)ₓ comprises any one of the antisense sequences present in Tables A through I.

It will be readily understood by those skilled in the art that the compounds of the present invention consist of a multitude of nucleotides which are linked through a covalent linkage; this covalent linkage may be a phosphodiester linkage, a phosphothioate linkage, or a combination of both, along the length of the nucleotide sequence of the individual strand. Other possible backbone modifications are described inter alia in U.S. Pat. Nos. 5,587,361; 6,242,589; 6,277,967; 6,326,358; 5,399,676; 5,489,677; and 5,596,086.

In particular embodiments, x and y may preferably be an integer between about 17 to about 27, most preferably from about 18 to about 23. In a particular embodiment of the compound of the invention, x may be equal to y (viz., x=y) and in preferred embodiments x=y=19, and x=y=21. In a particularly preferred embodiment x=y=19.

In one embodiment of the compound of the invention, Z and Z′ are both absent; in another embodiment Z or Z′ is present.

In one embodiment of the compound of the invention, all of the ribonucleotides of the compound are unmodified in their sugar residues.

In some embodiments of the compound of the invention, at least one ribonucleotide is modified in its sugar residue, preferably a modification at the 2′ position. The modification at the 2′ position is preferably selected from the group comprising amino, fluoro, methoxy, alkoxy and alkyl, and in a most preferred embodiment the modification at the 2′ position is methoxy (2′-0-methyl).

In some embodiments of the invention, alternating ribonucleotides are modified in both the antisense and the sense strands of the compound.

In particularly preferred embodiments of the invention, the antisense strand is phophorylated at the 5′terminus, and may or may not be phophorylated at the 3′terminus; and the sense strand may or may not be phophorylated at the 5′terminus and at the 3′terminus.

In another embodiment of the compound of the invention, the ribonucleotides at the 5′ and 3′ termini of the antisense strand are modified in their sugar residues, and the ribonucleotides at the 5′ and 3′ termini of the sense strand are unmodified in their sugar residues.

The invention further provides a vector capable of expressing any of the aforementioned oligoribonucleotides in a cell.

The invention also provides a composition comprising one or more of the compounds of the invention and a carrier, preferably a pharmaceutically acceptable carrier.

The invention also provides a composition comprising a carrier and one or more of the compounds of the invention in an amount effective to down-regulate expression in a cell of a gene of the TGase family which comprises a sequence substantially complementary to the sequence of (N)ₓ.

The invention also provides a composition comprising a carrier and one or more of the compounds of the invention in an amount effective to down-regulate expression in a cell of a one or more genes of the TGase family, in particular gene TGase 1, 3 5 or 7 which comprises a sequence substantially complementary to the sequence of (N)ₓ.

The invention also provides a method of inhibiting the expression of a TGase gene by at least 50% as compared to a control comprising contacting an mRNA transcript of the gene with one or more of the compounds of the invention.

In one embodiment the compound is inhibiting a gene of the TGase family, whereby the inhibition of TGase is selected from the group comprising inhibition of TGase function (which may be examined by an enzymatic assay or a binding assay with a known interactor of the native gene/polypeptide, inter alia), inhibition of TGase protein (which may be examined by Western blotting, ELISA or immuno-precipitation, inter alia) and inhibition of TGase mRNA expression (which may be examined by Northern blotting, quantitative RT-PCR, in-situ hybridisation or microarray hybridisation, inter alia).

In a particular embodiment the compound is inhibiting TGase I, whereby the inhibition of TGase I is selected from the group comprising inhibition of TGase I function (which may be examined by an enzymatic assay or a binding assay with a known interactor of the native gene/polypeptide, inter alia), inhibition of TGase I protein (which may be examined by Western blotting, ELISA or immuno-precipitation, inter alia) and inhibition of TGase I mRNA expression (which may be examined by Northern blotting, quantitative RT-PCR, in-situ hybridisation or microarray hybridisation, inter alia).

In other particular embodiment the compound is inhibiting TGase 3, 5 or 7, whereby the inhibition of TGase 3, 5 or 7,respectively is selected from the group comprising inhibition of TGase 3, 5 or 7 function respectively (which may be examined by an enzymatic assay or a binding assay with a known interactor of the native gene/polypeptide, inter alia), inhibition of TGase 3, 5 or 7 protein (which may be examined by Western blotting, ELISA or immuno-precipitation, inter alia) and inhibition of TGase 3, 5 or 7 mRNA expression (which may be examined by Northern blotting, quantitative RT-PCR, in-situ hybridisation or microarray hybridisation, inter alia).

The invention also provides a method of treating a patient suffering from fibrosis or a fibrosis related pathology comprising administering to the patient a composition of the invention in a therapeutically effective dose so as to thereby treat the patient.

The invention also provides a method of treating a patient suffering from a pathology related to aberrant cross-linking of cellular proteins via transglutaminase proteins comprising administering to the patient a composition of the invention in a therapeutically effective dose so as to thereby treat the patient.

The invention also provides a use of a therapeutically effective amount of one or more compounds of the invention for the preparation of a composition for promoting recovery in a patient suffering from fibrosis or a fibrosis related pathology or of pathology related to aberrant crosslinking of cellular proteins via transglutaminase enzymes. Fibrotic diseases or diseases in which fibrosis is evident (fibrosis related pathology) include both acute and chronic forms of fibrosis of organs, including all etiological variants of the following: pulmonary fibrosis, including interstitial lung disease and fibrotic lung disease, liver fibrosis, cardiac fibrosis including myocardial fibrosis, kidney fibrosis including chronic renal failure, skin fibrosis including scleroderma, keloids and hypertrophic scars; myelofibrosis (bone marrow fibrosis); all types of ocular scarring including proliferative vitreoretinopathy (PVR) and scarring resulting from surgery to treat cataract or glaucoma; inflammatory bowel disease of variable etiology, macular degeneration, Grave's ophthalmopathy, drug induced ergotism, keloid scars, scleroderma, psoriasis, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease, and collagenous colitis.

The compounds of the invention may be used to treat many other diseases and conditions apart from fibrotic diseases. Other indications may be ocular diseases including cataract and glaucoma, cardiovascular diseases especially cardiac hypertrophy, atherosclerosis/restenosis, neurological diseases, including polyglutamine diseases (such as Huntington's disease), spinobulbar muscular atrophy, dentatorubral-pallidoluysian atrophy and spinocerebellar ataxias (SCAB) 1, 2, 3, 6, 7 and 17, Alzheimer's disease and Parkinson's disease.

The compound may have homologs wherein up to two of the ribonucleotides in each terminal region a base is altered; the terminal region refers to the four terminal ribonucleotides e.g. refers to bases 1-4 and/or 16-19 in a 19-mer sequence and to bases 1-4 and/or 18-21 in a 21-mer sequence.

The preferred oligonucleotides of the invention are the siRNA oligonucleotides corresponding to TGase I which are set forth in Table A1. The most preferred oligonucleotides of the invention are human TGase I oligonucleotides of Table A1 in particular TGM1_{—}1 and TGM1_{—}11

The presently most preferred compound of the invention is a blunt-ended 19-mer oligonucleotide, i.e. x=y=19 and Z and Z′ are both absent; the oligonucleotide is phophorylated at the 5′ position of the antisense strand and at the 3′ position of the sense strand wherein alternating ribonucleotides are modified at the 2′ position in both the antisense and the sense strands, wherein the moiety at the 2′ position is methoxy (2′-0-methyl) and wherein the ribonucleotides at the 5′ and 3′ termini of the antisense strand are modified in their sugar residues, and the ribonucleotides at the 5′ and 3′ termini of the sense strand are unmodified in their sugar residues. The presently most preferred such compounds are such modified oligonucleotides comprising the sequences depicted in Table A1, in particular the human TGase I oligonucleotides of Table A1 especially TGM1_{—}1 and TGM1_{—}11.

In one aspect of the invention the oligonucleotide comprises a double-stranded structure, whereby such double-stranded structure comprises
a first strand and a second strand, whereby
the first strand comprises a first stretch of contiguous nucleotides and the second strand comprises a second stretch of contiguous nucleotides, whereby
the first stretch is either complementary or identical to a nucleic acid sequence coding for a TGase polypeptide and whereby the second stretch is either identical or complementary to a nucleic acid sequence coding for a TGase polypeptide.

In an embodiment the first stretch and/or the second stretch comprises from about 14 to 40 nucleotides, preferably about 18 to 30 nucleotides, more preferably from about 19 to 27 nucleotides and most preferably from about 19 to 23 nucleotides, in particular from about 19 to 21 nucleotides.

Additionally, further nucleic acids according to the present invention comprise at least 14 contiguous nucleotides of any one of the SEQ. ID. NO. 3 to last SEQ. ID. NO (any of the 19-mers or 21-mers in Tables A-I), and more preferably 14 contiguous nucleotide base pairs at any end of the double-stranded structure comprised of the first stretch and second stretch as described above.

The term “treatment” as used herein refers to administration of a therapeutic substance effective to ameliorate symptoms associated with a disease, to lessen the severity or cure the disease, or to prevent the disease from occurring.

In a particular embodiment, the administration comprises intravenous administration. In another particular embodiment the administration comprises topical or local administration such as for example administration to the eye via intravitreous or anterior chamber injection.

Additionally, the present invention provides a method of regulating a pathology or disease (as recited above) in a patient in need of such treatment by administering to a patient a therapeutically effective dose of at least inhibitor e.g. at least one antisense (AS) oligonucleotide or at least one siRNA against the nucleic acid sequences or a dominant negative peptide directed against the TGase sequences or TGase proteins or an antibody directed against the TGase polypeptide. Additionally the present invention provides a method of treating a patient suffering from a disorder comprising administering to the patient one or more inhibitors of human TGase in a therapeutically effective dose so as to thereby treat the patient. In a preferred method the inhibitors are siRNAs.

Delivery: Delivery systems aimed specifically at the enhanced and improved delivery of siRNA into mammalian cells have been developed, see, for example, Shen et al (FEBS letters 539: 111-114 (2003)), Xia et al., Nature Biotechnology 20: 1006-1010 (2002), Reich et al., Molecular Vision 9: 210-216 (2003), Sorensen et al. (J. Mol. Biol. 327: 761-766 (2003), Lewis et al., Nature Genetics 32: 107-108 (2002) and Simeoni et al., Nucleic Acids Research 31, 11: 2717-2724 (2003). siRNA has recently been successfully used for inhibition in primates; for further details see Tolentino et al, Retina 24(1) February 2004 I 132-138. Respiratory formulations for siRNA are described in U.S. patent application No. 2004/0063654 of Davis et al. Cholesterol-conjugated siRNAs (and other steroid and lipid conjugated siRNAs) can been used for delivery see Soutschek et al Nature 432: 173-177(2004) Therapeutic silencing of an endogenous gene by systemic administration of modified siRNAs; and Lorenz et al. Bioorg. Med. Chemistry. Lett. 14:4975-4977 (2004) Steroid and lipid conjugates of siRNAs to enhance cellular uptake and gene silencing in liver cells.

The siRNAs or pharmaceutical compositions of the present invention are administered and dosed in accordance with good medical practice, taking into account the clinical condition of the individual patient, the disease to be treated, the site and method of administration, scheduling of administration, patient age, sex, body weight and other factors known to medical practitioners.

The “therapeutically effective dose” for purposes herein is thus determined by such considerations as are known in the art. The dose must be effective to achieve improvement including but not limited to improved survival rate or more rapid recovery, or improvement or elimination of symptoms and other indicators as are selected as appropriate measures by those skilled in the art. The compounds of the present invention can be administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Liquid forms may be prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles. In addition, under certain circumstances the compositions for use in the novel treatments of the present invention may be formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention and they include liposomes and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art. In one specific embodiment of this invention topical and transdermal formulations are particularly preferred.

In general, the active dose of compound for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-4 weeks or longer. Treatment for many years or even lifetime treatment is also envisaged for some of the indications disclosed herein.

The present invention also provides for a process of preparing a pharmaceutical composition which comprises:
obtaining at one or more double stranded compound of the invention; and admixing said compound with a pharmaceutically acceptable carrier.

The present invention also provides for a process of preparing a pharmaceutical composition which comprises admixing a compound of the present invention with a pharmaceutically acceptable carrier.

In a preferred embodiment, the compound used in the preparation of a pharmaceutical composition is admixed with a carrier in a pharmaceutically effective amount. In a particular embodiment the compound of the present invention is conjugated to a steroid or to a lipid or to another suitable molecule; in a specific example the conjugation is to cholesterol.

Modifications or analogs of nucleotides can be introduced to improve the therapeutic properties of the nucleotides. Improved properties include increased nuclease resistance and/or increased ability to permeate cell membranes.

Accordingly, the present invention also includes all analogs of, or modifications to, a oligonucleotide of the invention that does not substantially affect the function of the polynucleotide or oligonucleotide. In a preferred embodiment such modification is related to the base moiety of the nucleotide, to the sugar moiety of the nucleotide and/or to the phosphate moiety of the nucleotide.

In embodiments of the invention, the nucleotides can be selected from naturally occurring or synthetically modified bases. Naturally occurring bases include adenine, guanine, cytosine, thymine and uracil. Modified bases of the oligonucleotides include inosine, xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogs of nucleotides can be prepared wherein the structures of the nucleotides are fundamentally altered and are better suited as therapeutic or experimental reagents. An example of a nucleotide analog is a peptide nucleic acid (PNA) wherein the deoxyribose (or ribose) phosphate backbone in DNA (or RNA) is replaced with a polyamide backbone similar to that found in peptides. PNA analogs have been shown to be resistant to degradation by enzymes and to have extended lives in vivo and in vitro. Further, PNAs have been shown to bind more strongly to a complementary DNA sequence than to a DNA molecule. This observation is attributed to the lack of charge repulsion between the PNA strand and the DNA strand. Other modifications that can be made to oligonucleotides include polymer backbones, cyclic backbones, or acyclic backbones.

In one embodiment the modification is a modification of the phosphate moiety, whereby the modified phosphate moiety is selected from the group comprising phosphothioate.

The compounds of the present invention can be synthesized by any of the methods that are well-known in the art for synthesis of ribonucleic (or deoxyribonucleic) oligonucleotides. Such synthesis is, among others, described in Beaucage S. L. and Iyer R. P., Tetrahedron 1992; 48: 2223-2311, Beaucage S. L. and Iyer R. P., Tetrahedron 1993; 49: 6123-6194 and Caruthers M. H. et. al., Methods Enzymol. 1987; 154: 287-313, the synthesis of thioates is, among others, described in Eckstein F., Annu. Rev. Biochem. 1985; 54: 367-402, the synthesis of RNA molecules is described in Sproat B., in Humana Press 2005 Edited by Herdewijn P.; Kap. 2: 17-31 and respective downstream processes are, among others, described in Pingoud A. et. al., in IRL Press 1989 Edited by Oliver R. W. A.; Kap. 7: 183-208 and Sproat B., in Humana Press 2005 Edited by Herdewijn P.; Kap. 2: 17-31 (supra).

Other synthetic procedures are known in the art e.g. the procedures as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and these procedures may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

The oligonucleotides of the present invention can be synthesized separately and joined together post-synthetically, for example, by ligation (Moore et al., 1992, Science 256, 9923; Draper et al., International PCT publication No. WO93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

It is noted that a commercially available machine (available, inter alia, from Applied Biosystems) can be used; the oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present invention, two or more such sequences can be synthesized and linked together for use in the present invention.

The compounds of the invention can also be synthesized via a tandem synthesis methodology, for example as described in US patent application publication No. US2004/0019001 (McSwiggen) wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker.

The compounds of the present invention can be delivered either directly or with viral or non-viral vectors. When delivered directly the sequences are generally rendered nuclease resistant. Alternatively the sequences can be incorporated into expression cassettes or constructs such that the sequence is expressed in the cell as discussed herein below. Generally the construct contains the proper regulatory sequence or promoter to allow the sequence to be expressed in the targeted cell. Vectors optionally used for delivery of the compounds of the present invention are commercially available, and may be modified for the purpose of delivery of the compounds of the present invention by methods known to one of skill in the art.

It is also envisaged that a long oligonucleotide (typically 25-500 nucleotides in length) comprising one or more stem and loop structures, where stem regions comprise the sequences of the oligonucleotides of the invention, may be delivered in a carrier, preferably a pharmaceutically acceptable carrier, and may be processed intracellularly by endogenous cellular complexes (e.g. by DROSHA and DICER as described above) to produce one or more smaller double stranded oligonucleotides (siRNAs) which are oligonucleotides of the invention. This oligonucleotide can be termed a tandem shRNA construct. It is envisaged that this long oligonucleotide is a single stranded oligonucleotide comprising one or more stem and loop structures, wherein each stem region comprises a sense and corresponding, antisense siRNA sequence of a TGase gene. In particular, it is envisaged that this oligonucleotide comprises sense and antisense siRNA sequences as depicted in any one of Tables A through I, which are located below in Example 1.

As used herein, the term “polypeptide” refers to, in addition to a polypeptide, an oligopeptide, peptide and a full protein.

Animal model systems: Testing the active siRNAs of the invention may be done in predictive animal models. Several models for kidney fibrosis are described in Example 3.

Two models of liver fibrosis in rats are the Bile Duct Ligation (BDL) with sham operation as controls, and CCl₄ poisoning, with olive oil fed animals as controls, as described in the following references: Lotersztajn S, Julien B, Teixeira-Clerc F, Grenard P, Mallat A, Hepatic Fibrosis: Molecular Mechanisms and Drug Targets. Annu Rev Pharmacol Toxicol. 2004 Oct. 7; Uchio K, Graham M, Dean N M, Rosenbaum J, Desmouliere A., Down-regulation of connective tissue growth factor and type I collagen mRNA expression by connective tissue growth factor antisense oligonucleotide during experimental liver fibrosis. Wound Repair Regen. 2004 January-February; 12(1):60-6; and Xu X Q, Leow C K, Lu X, Zhang X, Liu J S, Wong W H, Asperger A, Deininger S, Eastwood Leung H C., Molecular classification of liver cirrhosis in a rat model by proteomics and bioinformatics Proteomics. 2004 October; 4(10):3235-45.

Models for ocular scarring are well known in the art e.g. Sherwood M B et al., J Glaucoma. 2004 October; 13(5):407-12. A new model of glaucoma filtering surgery in the rat; Miller M H et al., Ophthalmic Surg. 1989 May; 20(5):350-7. Wound healing in an animal model of glaucoma fistulizing surgery in the rabbit; vanBockxmeer F M et al., Retina. 1985 Fall-Winter; 5(4): 239-52. Models for assessing scar tissue inhibitors; Wiedemann P et al., J Pharmacol Methods. 1984 August; 12(1): 69-78. Proliferative vitreoretinopathy: the rabbit cell injection model for screening of antiproliferative drugs.

Models of cataract are described in the following publications: The role of Src family kinases in cortical cataract formation. Zhou J, Menko A S. Invest Ophthalmol Vis Sci. 2002 July; 43(7):2293-300; Bioavailability and anticataract effects of a topical ocular drug delivery system containing disulfiram and hydroxypropyl-beta-cyclodextrin on selenite-treated rats. Wang S, Li D, Ito Y, Nabekura T, Wang S, Zhang J, Wu C. Curr Eye Res. 2004 July; 29(1):51-8; and Long-term organ culture system to study the effects of UV-Airradiation on lens transglutaminase. Weinreb O, Dovrat A.; Curr Eye Res. 2004 July; 29(1):51-8.

Antibody Production

By the term “antibody” as used in the present invention is meant both poly- and mono-clonal complete antibodies as well as fragments thereof, such as Fab, F(ab′)2, miniantibody (minibody) and Fv, which are capable of binding the epitopic determinant. These antibody fragments retain the ability to selectively bind with its antigen or receptor and are exemplified as follows, inter alia:
(1) Fab, the fragment which contains a monovalent antigen-binding fragment of an antibody molecule can be produced by digestion of whole antibody with the enzyme papain to yield a light chain and a portion of the heavy chain;
(2) (Fab′)2, the fragment of the antibody that can be obtained by treating whole antibody with the enzyme pepsin without subsequent reduction; F(ab′2) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.
(5) Miniantibody (minibody), defined as a genetically engineered molecule containing variable regions of the light chain and variable regions of the heavy chain (scFv or single chain variable fragment) linked by a suitable polypeptide linker combined with constant Fc regions.

Such fragments having antibody functional activity can be prepared by methods known to those skilled in the art (e.g. Bird et al. (1988) Science 242:423-426)

Conveniently, antibodies may be prepared against the immunogen or portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art, as described generally in Harlow and Lane (1988), Antibodies: A Laboratory Manual, Cold Spring Harbor Laboratory, Cold Spring Harbor, N.Y., and Borrebaeck (1992), Antibody Engineering—A Practical Guide, W.H. Freeman and Co., NY.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific; that is, the sera can be absorbed against related immunogens so that no cross-reactive antibodies remain in the sera, rendering it monospecific.

For producing monoclonal antibodies the technique involves hyperimmunization of an appropriate donor with the immunogen, generally a mouse, and isolation of splenic antibody-producing cells. These cells are fused to an immortal cell, such as a myeloma cell, to provide a fused cell hybrid that is immortal and secretes the required antibody. The cells are then cultured, in bulk, and the monoclonal antibodies harvested from the culture media for use.

For producing recombinant antibody see generally Huston et al. (1991) “Protein engineering of single-chain Fv analogs and fusion proteins” in Methods in Enzymology (J J Langone, ed., Academic Press, New York, N.Y.) 203:46-88; Johnson and Bird (1991) “Construction of single-chain Fvb derivatives of monoclonal antibodies and their production in Escherichia coli in Methods in Enzymology (J J Langone, ed.; Academic Press, New York, N.Y.) 203:88-99; Mernaugh and Mernaugh (1995) “An overview of phage-displayed recombinant antibodies” in Molecular Methods In Plant Pathology (R P Singh and U S Singh, eds.; CRC Press Inc., Boca Raton, Fla.:359-365). In particular scFv antibodies are described in WO 2004/007553 (Tedesco and Marzari). Additionally, messenger RNAs from antibody-producing B-lymphocytes of animals, or hybridoma can be reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. (For a general discussion of conjugation of fluorescent or enzymatic moieties see Johnstone & Thorpe (1982.), Immunochemistry in Practice, Blackwell Scientific Publications, Oxford). The binding of antibodies to a solid support substrate is also well known in the art (for a general discussion, see Harlow & Lane (1988) Antibodies: A Laboratory Manual, Cold Spring Harbor Laboratory Publications, New York; and Borrebaeck (1992), Antibody Engineering—A Practical Guide, W.H. Freeman and Co.). The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹⁴C and iodination.

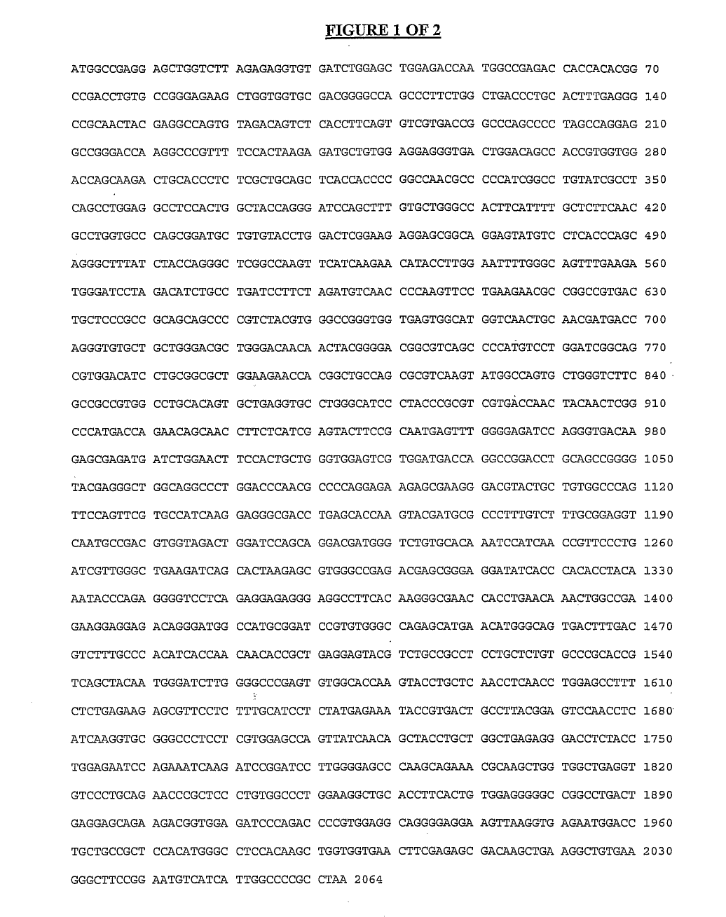

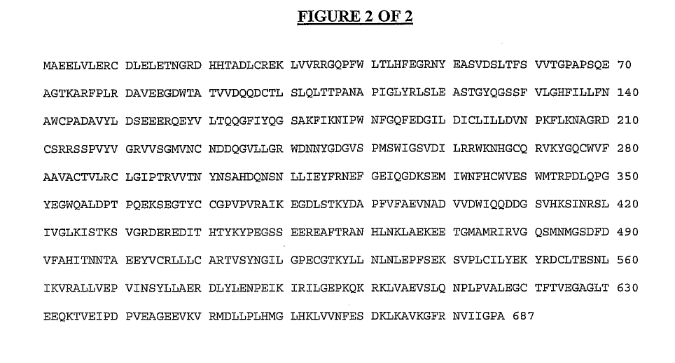

Additional compounds which are also considered to be useful in the treatment of the diseases and disorders discussed herein may be antisense DNA molecules (which can be generated using the sequence in FIG. 1 by methods known in the art), catalytic RNAs such as ribozymes, polypeptides such as dominant negative peptides (which can be generated using the sequence in FIG. 2 by methods known in the art) or other polypeptide inhibitors. Antisense DNA molecules which comprise the siRNA sequences disclosed herein (with the appropriate nucleic acid modifications stemming from the differences between DNA and RNA) are particularly desirable and may be used in the same capacity as their corresponding siRNAs for all uses and methods disclosed herein.

Screening:

The compounds and compositions of the present invention may be used in a screening assay for identifying and isolating compounds which modulate the activity of Transglutaminases, in particular TGases I, 3, 5, 7 in particular, compounds which modulate TGase crosslinking activity, fibrotic disease, ocular scarring, cataract and glaucoma. The compounds to be screened comprise inter alia substances such as small chemical molecules, antibodies especially neutralizing antibodies, antisense oligonucleotides, antisense DNA or RNA molecules, polypeptides and dominant negatives, and expression vectors.

The inhibitory activity of the compounds of the present invention on TGase gene expression may be used to determine the interaction of an additional compound with the TGase gene or polypeptide, e.g., if the additional compound competes with the oligoribonucleotides of the present invention for TGase inhibition, or if the additional compound rescues said inhibition. The inhibition or activation can be tested by various means, such as, inter alia, assaying for the TGase mRNA or polypeptide, a product of the activity of the TGase polypeptide, radiolabeled or fluorescent competition assays.

### Methods

### General Methods in Molecular Biology

Standard molecular biology techniques known in the art and not specifically described were generally followed as in Sambrook et al., Molecular Cloning: A Laboratory Manual, Cold Spring Harbor Laboratory Press, New York (1989), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In situ (In cell) PCR in combination with Flow Cytometry can be used for detection of cells containing specific DNA and mRNA sequences (Testoni et al., 1996, Blood 87:3822.) Methods of performing RT-PCR are also well known in the art.

The present invention is illustrated in detail below with reference to Examples, but is not to be construed as being limited thereto.

### Examples

### Example 1

### Generation of Sequences for Active siRNA Compounds

Using proprietary algorithms and the known sequence of each TGase gene, the sequences of many potential siRNAs were generated and subsequently selected. These are shown in Tables A through I which follow below.

Tables A through I describe many 19-mer and 21-mer siRNAs which inhibit TGases I, 3 5 and 7.

TGase 1

Table A1 shows the fourteen preferred 19-mer siRNAs. Ten of these have been synthesized and tested for activity, as shown in Table A2. Nine of these (all except TGM1_{—}3) showed activity against one or both of rabbit or human TGase 1 expression. TGM1_{—}1 and TGM1_{—}11 showed the best activity against both rabbit and human TGase 1. Thus these two siRNAs are preferred for use in animal (rabbit) studies with the expectation that they will also be active in humans.

Table B below shows additional 19-mer siRNAs which have been generated by the proprietary algorithms but not yet synthesized. Table C below shows additional 21-mer siRNAs which have been generated by the proprietary algorithms

TGases 3, 5 and 7.

Tables D and E below describe 19 and 21-mer siRNAs, respectively, corresponding to TGase 3. Similarly Tables F and G, describe respectively 19 and 21-mer siRNAs corresponding to TGase 5, and Tables H and I describe respectively 19 and 21-mer siRNAs corresponding to TGase7.

TABLE A1
TGase I: 19-mers (selected)

| | | 0ligo | | | human | mouse | rabbit |
|---|---|---|---|---|---|---|---|
| name | Method | Length | Sense siRNA | AntiSense siRNA | 4507474 | 31982704 | 166769 |
| (TGM1_8) | Single Sp | 19 | GCAGGAGUAUGUUCUUAAU | ATTAAGAACATACTCCTGC | [872-890] | — | — |
| (TGM1_1) | Single Sp | 19 | GCAGCAAACCCAAUGUGUA | TACACATTGGGTTTGCTGC | [1771-1789] | — | [1794-1812] |
| (TGM1_9) | Cross Sp | 19 | CUCCCUGGAUGACAAUGGA | TCCATTGTCATCCAGGGAG | [1070-1088] | [1146-1164] | [1113-1131] |
| (TGM1_2) | Cross Sp | 19 | GAACUCCCUGGAUGACAAU | ATTGTCATCCAGGGAGTTC | [1067-1085] | [1143-1161] | [1110-1128] |
| (TGM1_10) | Cross Sp | 19 | GGAGAUCCUGCUUAGCUAC | GTAGCTAAGCAGGATCTCC | [1157-1175] | — | [1200-1218] |
| (TGM1_11) | Cross Sp | 19 | UGGAGAUCCUGCUUAGCUA | TAGCTAAGCAGGATCTCCA | [1156-1174] | — | [1199-1217] |
| (TGM1_12) | Cross Sp | 19 | GUGGAGAUCCUGCUUAGCU | AGCTAAGCAGGATCTCCAC | [1155-1173] | — | [1198-1216] |
| (TGM1_3) | Cross Sp | 19 | GGCAGCAAACCCAAUGUGU | ACACATTGGGTTTGCTGCC | [1770-1788] | — | [1793-1811] |
| (TGM1_13) | Cross Sp | 19 | CCAUGCUGCUCAAUGUCUC | GAGACATTGAGCAGCATGG | [2071-2089] | — | [2094-2112] |
| (TGM1_4) | Cross Sp | 19 | GUGACAAGGUGUACUGGCA | TGCCAGTACACCTTGTCAC | [1579-1597] | — | [1622-1840] |
| (TGM1_14) | Cross Sp | 19 | GGUGAAUAGUGACAAGGUG | CACCTTGTCACTATTCACC | [1571-1589] | — | [1614-1632] |
| (TGM1_5) | Single Sp | 19 | CCAUGGACAUCUACUUUGA | TCAAAGTAGATGTCCATGG | — | — | [1358-1376] |
| (TGM1_6) | Single Sp | 19 | CUGGAACUAUGGCCAGUUU | AAACTGGCCATAGTTCCAG | — | — | [984-1002] |
| (TGM1_7) | Single Sp | 19 | CCAUCAUUGGCAAAUUCCA | TGGAATTTGCCAATGATGG | — | — | [767-785] |

| | rat | Cow |
|---|---|---|
| name | 13928911 | 38641347 |
| (TGM1_8) | — | — |
| (TGM1_1) | — | — |
| (TGM1_9) | [1117-1135] | — |
| (TGM1_2) | [1114-1132] | — |
| (TGM1_10) | [1204-4222] | — |
| (TGM1_11) | [1203-1221] | — |
| (TGM1_12) | [1202-1220] | — |
| (TGM1_3) | — | [1376-1394] |
| (TGM1_13) | — | — |
| (TGM1_4) | — | — |
| (TGM1_14) | — | — |
| (TGM1_5) | — | — |
| (TGM1_6) | — | — |
| (TGM1_7) | — | — |

TABLE A2

Results of inhibition of TGase 1
expression by ten of the selected siRNAs

| | | |
|---|---|---|
| | | % Inhibition of |
| | % Inhibition of rabbit | human TGase 1 |
| | TGase 1 expression | expression (tested |
| | (tested 48hr following | 48hr following |
| | cotransfection with | transfection to |
| | rabbit TGase 1 into 293 | SKBR3 human cell |
| Sense siRNA | cell line) | line) |

| | | | |
|---|---|---|---|
| TGM1_11 | UGGAGAUCCUGCUUAGCUA | 95 | 85 |
| TGM1_12 | GUGGAGAUCCUGCUUAGCU | 0 | 55 |
| TGM1_5 | CCAUGGACAUCUACUUUGA | 60 | 90 |
| TGM1_13 | CCAUGCUGCUCAAUGUCUC | 40 | 90 |
| TGM1_6 | CUGGAACUAUGGCCAGUUU | 30 | 10 |
| TGM1_14 | GGUGAAUAGUGACAAGGUG | 50 | 70 |
| TGM1_8 | GCAGGAGUAUGUUCUUAAU | 0 | 94 |
| TGM1_1 | GCAGCAAACCCAAUGUGUA | 85 | 90 |
| TGM1_10 | GGAGAUCCUGCUUAGCUAC | 20 | 60 |
| TGM1_3 | GGCAGCAAACCCAAUGUGU | 0 | 0 |

TABLE B
TGase 1:19-mers

| | Oligo | | | human | mouse | rabbit | rat | CoW GI |
|---|---|---|---|---|---|---|---|---|
| Method | Length | Sense siRNA | AntiSense siRNA | 4507474 | 31982704 | 165769 | 13928911 | 31343554 |

| | | | | | | | | |
|---|---|---|---|---|---|---|---|---|
| Cross | 19 | AUAAGCACCCAGAAGGCUC | GAGCCUUCUGGGUGCUUAU | 1711-1729 | 1787-1805 | 1754-1772 | 1758-1776 | — |
| Sp | | | | | | | | |
| Cross | 19 | CUAUAAGCACCCAGAAGGC | GCCUUCUGGGUGCUUAUAG | 1709-1727 | 1785-1803 | 1752-1770 | 1756-1774 | — |
| Sp | | | | | | | | |
| Cross | 19 | UCUAUAAGCACCCAGAAGG | CCUUCUGGGUGCUUAUAGA | 1708-1726 | 1784-1802 | 1751-1769 | 1755-1773 | — |
| Sp | | | | | | | | |
| Single | 19 | GCAAUGAGAUCUACAUCCU | AGGAUGUAGAUCUCAUUGC | 799-817 | 875-893 | 842-860 | 846-864 | — |
| Sp | | | | | | | | |
| Single | 19 | GGAGUAUGUUCUUAAUGAG | CUCAUUAAGAACAUACUCC | 875-893 | — | — | — | — |
| Sp | | | | | | | | |
| Cross | 19 | GGUGAACUCCCUGGAUGAC | GUCAUCCAGGGAGUUCACC | 1064-1082 | 1140-1158 | 1107-1125 | 1111-1129 | — |
| Sp | | | | | | | | |
| Cross | 19 | AUGGUGAACUCCCUGGAUG | CAUCCAGGGAGUUCACCAU | 1062-1080 | 1138-1156 | 1105-1123 | 1109-1127 | — |
| Sp | | | | | | | | |
| Cross | 19 | CUCUGCCAUGGUGAACUCC | GGAGUUCACCAUGGCAGAG | 1055-1073 | 1131-1149 | 1098-1116 | 1102-1120 | — |
| Sp | | | | | | | | |
| Cross | 19 | CAUGGUGAACUCCCUGGAU | AUCCAGGGAGUUCACCAUG | 1061-1079 | 1137-1155 | 1104-1122 | 1108-1126 | — |
| Sp | | | | | | | | |
| Cross | 19 | UAUAAGCACCCAGAAGGCU | AGCCUUCUGGGUGCUUAUA | 1710-1728 | 1786-1804 | 1753-1771 | 1757-1775 | — |
| Sp | | | | | | | | |
| Cross | 19 | AACUCCCUGGAUGACAAUG | CAUUGUCAUCCAGGGAGUU | 1068-1086 | 1144-1162 | 1111-1129 | 1115-1133 | — |
| Sp | | | | | | | | |
| Cross | 19 | CUGCCAUGGUGAACUCCCU | AGGGAGUUCACCAUGGCAG | 1057-1075 | 1133-1151 | 1100-1118 | 1104-1122 | — |
| Sp | | | | | | | | |
| Cross | 19 | UCUGCCAUGGUGAACUCCC | GGGAGUUCACCAUGGCAGA | 1056-1074 | 1132-1150 | 1099-1117 | 1103-1121 | — |
| Sp | | | | | | | | |
| Cross | 19 | UGGUGAACUCCCUGGAUGA | UCAUCCAGGGAGUUCACCA | 1063-1081 | 1139-1157 | 1106-1124 | 1110-1128 | — |
| Sp | | | | | | | | |
| Cross | 19 | AGCAGUGGCAUCUUCUGCU | AGCAGAAGAUGCCACUGCU | 1476-1494 | 1552-1570 | 1519-1537 | 1523-1541 | — |
| Sp | | | | | | | | |
| Cross | 19 | CCUUUGACCCCCGCAAUGA | UCAUUGCGGGGGUCAAAGG | 787-805 | 863-881 | 830-848 | 834-852 | — |
| Sp | | | | | | | | |
| Cross | 19 | UCCCUGGAUGACAAUGGAG | CUCCAUUGUCAUCCAGGGA | 1071-1089 | 1147-1165 | 1114-1132 | 1118-1136 | — |
| Sp | | | | | | | | |
| Cross | 19 | CCAUGGUGAACUCCCUGGA | UCCAGGGAGUUCACCAUGG | 1060-1078 | 1136-1154 | 1103-1121 | 1107-1125 | — |
| Sp | | | | | | | | |
| Cross | 19 | CCCUGGAUGACAAUGGAGU | ACUCCAUUGUCAUCCAGGG | 1072-1090 | 1148-1166 | 1115-1133 | 1119-1137 | — |
| Sp | | | | | | | | |
| Single | 19 | GGCAGCCUUUCCAUAUGCU | AGCAUAUGGAAAGGCUGCC | 511-529 | — | 554-566 | — | — |
| Sp | | | | | | | | |
| Cross | 19 | UCUCUGCCAUGGUGAACUC | GAGUUCACCAUGGCAGAGA | 1054-1072 | 1130-1148 | 1097-1115 | 1101-1119 | — |
| Sp | | | | | | | | |
| Cross | 19 | UGAACUCCCUGGAUGACAA | UUGUCAUCCAGGGAGUUCA | 1066-1084 | 1142-1160 | 1109-1127 | 1113-1131 | — |
| Sp | | | | | | | | |
| Single | 19 | CUGUGAUGCUGAUCAAUCA | UGAUUGAUCAGCAUCACAG | 1870-1888 | — | — | — | — |
| Sp | | | | | | | | |
| Cross | 19 | ACUCCCUGGAUGACAAUGG | CCAUUGUCAUCCAGGGAGU | 1069-1087 | 1145-1163 | 1112-1130 | 1116-1134 | — |
| Sp | | | | | | | | |
| Single | 19 | CCAGUGGGCAGAAUCUGAA | UUCAGAUUCUGCCCACUGG | 679-697 | — | — | — | — |
| Sp | | | | | | | | |
| Single | 19 | AGAUCUACAUCOUCUUCAA | UUGAAGAGGAUGUAGAUCU | 805-823 | 881-899 | 850-866 | 852-870 | — |
| Sp | | | | | | | | |
| Cross | 19 | UGCUCAAUGUCUCAGGCCA | UGGCCUGAGACAUUGAGCA | 2077-2095 | 2153-2171 | 2100-2118 | 2124-2142 | — |
| Sp | | | | | | | | |
| Cross | 19 | GUGAACUCCCUGGAUGACA | UGUCAUCCAGGGAGUUCAC | 1065-1083 | 1141-1159 | 1108-1126 | 1112-1130 | — |
| Sp | | | | | | | | |
| Single | 19 | CCAUCAUCGGCAAGUUUCA | UGAAACUUGCCGAUGAUGG | 724-742 | 800-818 | — | 771-789 | — |
| Sp | | | | | | | | |
| Cross | 19 | UGCCAUGGUGAACUCCCUG | CAGGGAGUUCACCAUGGCA | 1058-1076 | 1134-1152 | 1101-1119 | 1105-1123 | — |
| Sp | | | | | | | | |
| Cross | 19 | GCAGUGGCAUCUUCUGCUG | CAGCAGAAGAUGCCACUGC | 1477-1495 | 1553-1571 | 1520-1538 | 1524-1542 | — |
| Sp | | | | | | | | |
| Single | 19 | GCAUCACCCUUGAGUUACU | AGUAACUCAAGGGUGAUGC | 565-583 | — | — | — | — |
| Sp | | | | | | | | |
| Cross | 19 | AGAUCCUGCUUAGCUACCU | AGGUAGCUAAGCAGGAUCU | 1159-1177 | — | 1202-1220 | 1206-1224 | — |
| Sp | | | | | | | | |
| Cross | 19 | ACUGUCACCAACUUCAACU | AGUUGAAGUUGGUGACAGU | 1269-1287 | — | 1312-1330 | 1316-1334 | — |
| Sp | | | | | | | | |
| Single | 19 | CCAGACCUCUCUCUGACAU | AUGUCAGAGAGAGGUCUGG | 2151-2161 | — | — | 2198-2216 | — |
| Sp | | | | | | | | |
| Single | 19 | CGAAGACUGGCGACAAGAA | UUCUUGUCGCCAGUCUUCG | — | — | — | 907-925 | — |
| Sp | | | | | | | | |
| Cross | 19 | CUGUCACCAACUUCAACUC | GAGUUGAAGUUGGUGACAG | 1270-1288 | — | 1313-1331 | 1317-1335 | — |
| Sp | | | | | | | | |
| Single | 19 | CCACACCGAUGAGUUUGAA | UUCAAACUCAUCGGUGUGG | — | — | 510-527 | 514-532 | — |
| Sp | | | | | | | | |
| Cross | 19 | GAGAUCCUGCUUAGCUACC | GGUAGCUAAGCAGGAUCUC | 1158-1176 | — | 1201-1219 | 1205-1223 | — |
| Sp | | | | | | | | |
| Single | 19 | CUCCCCUAAUGCCAUCAUU | AAUGAUGGCAUUAGGGGAG | — | — | — | 760-778 | — |
| Sp | | | | | | | | |
| Single | 19 | GGCAGCCCUUCCACAUAAU | AUUAUGUGGAAGGGCUGCC | — | — | — | 558-576 | — |
| Sp | | | | | | | | |
| Single | 19 | CCAUCAUUGGCAAGUUUCA | UGAAACUUGCCAAUGAUGG | 724-742 | — | 767-779 | 771-789 | — |
| Sp | | | | | | | | |
| Single | 19 | GAAGACUGGCGACAAGAAU | AUUCUUGUCGCCAGUCUUC | — | — | — | 908-926 | — |
| Sp | | | | | | | | |
| Single | 19 | CGAUGAGUUUGAAUAUGAC | GUCAUAUUCAAACUCAUCG | — | — | 517-533 | 520-538 | — |
| Sp | | | | | | | | |
| Cross | 19 | CAGAGGACAUUGUGUACGU | ACGUACACAAUGUCCUCUG | 835-853 | — | 878-896 | — | — |
| Sp | | | | | | | | |
| Cross | 19 | AAUGUCUCAGGCCACGUCA | UGACGUGGCCUGAGACAUU | 2082-2100 | — | 2105-2123 | — | — |
| Sp | | | | | | | | |
| Cross | 19 | AUGCGGCAGAUGACGACUG | CAGUCGUCAUCUGCCGCAU | 244-262 | — | 317-335 | — | — |
| Sp | | | | | | | | |
| Cross | 19 | AGUGACAAGGUGUACUGGC | GCCAGUACACCUUGUCACU | 1578-1596 | — | 1621-1639 | — | — |
| Sp | | | | | | | | |
| Cross | 19 | CUGGUGCCCAGAGGACAUU | AAUGUCCUCUGGGCACCAG | 827-845 | — | 870-888 | — | — |
| Sp | | | | | | | | |
| Cross | 19 | GCGUGGAGAUCCUGCUUAG | CUAAGCAGGAUCUCCACGC | 1153-1171 | — | 1196-1214 | — | — |
| Sp | | | | | | | | |
| Cross | 19 | UGGCCAGUGCUGGGUCUUU | AAAGACCCAGCACUGGCCA | 1202-1220 | — | 1245-1263 | — | — |
| Sp | | | | | | | | |
| Cross | 19 | GAAUAGUGACAAGGUGUAC | GUACACCUUGUCACUAUUC | 1574-1592 | — | 1617-1635 | — | — |
| Sp | | | | | | | | |
| Cross | 19 | AAUGAGUCUGGGAGAAUUU | AAAUUCUCCCAGACUCAUU | 888-906 | — | 931-949 | — | — |
| Sp | | | | | | | | |
| Cross | 19 | AUUGCCAGCUUGGACAGCC | GGCUGUCCAAGCUGGCAAU | 2379-2397 | — | 2402-2420 | — | — |
| Sp | | | | | | | | |
| Cross | 19 | GGAUGAUGGCAGCUUCAAG | CUUGAAGCUGCCAUCAUCC | 1604-1622 | — | 1647-1665 | — | — |
| Sp | | | | | | | | |
| Cross | 19 | AGCAAUGGGACUGGAGUCA | UGACUCCAGUCCCAUUGCU | 2547-2565 | — | 2567-2585 | — | — |
| Sp | | | | | | | | |
| Cross | 19 | AUCGGAAACAACCCCGAGG | CCUCGGGGUUGUUUCCGAU | 585-603 | — | 628-646 | — | — |
| Sp | | | | | | | | |
| Cross | 19 | GAUGGCAGCUUCAAGAUUG | CAAUCUUGAAGCUGCCAUC | 1608-1626 | — | 1651-1669 | — | — |
| Sp | | | | | | | | |
| Cross | 19 | CAUGCUGCUCMUGUCUCA | UGAGACAUUGAGCAGCAUG | 2072-2090 | — | 2095-2113 | — | — |
| Sp | | | | | | | | |
| Cross | 19 | AGGACAUUGUGUACGUGGA | UCCACGUACACAAUGUCCU | 838-856 | — | 881-899 | — | — |
| Sp | | | | | | | | |
| Cross | 19 | AUGAUGGCAGCUUCAAGAU | AUCUUGAAGCUGCCAUCAU | 1606-1624 | — | 1649-1667 | — | — |
| Sp | | | | | | | | |
| Cross | 19 | CAAUGUCUCAGGCCACGUC | GACGUGGCCUGAGACAUUG | 2081-2099 | — | 2104-2122 | — | — |
| Sp | | | | | | | | |
| Cross | 19 | UGGAUGACAAUGGAGUCCU | AGGACUCCAUUGUCAUCCA | 1075-1093 | — | 1118-1136 | — | — |
| Sp | | | | | | | | |
| Cross | 19 | UCGGAAACAACCCCGAGGU | ACCUCGGGGUUGUUUCCGA | 586-604 | — | 629-647 | — | — |
| Sp | | | | | | | | |
| Cross | 19 | AUUGUGUACGUGGACCAUG | CAUGGUCCACGUACACAAU | 843-861 | — | 886-904 | — | — |
| Sp | | | | | | | | |
| Cross | 19 | AUGCUGCUCAAUGUCUCAG | CUGAGACAUUGAGCAGCAU | 2073-2091 | — | 2096-2114 | — | — |
| Sp | | | | | | | | |
| Cross | 19 | UGUACGUGGACCAUGAGGA | UCCUCAUGGUCCACGUACA | 847-865 | — | 890-908 | — | — |
| Sp | | | | | | | | |
| Cross | 19 | AUAGUGACAAGGUGUACUG | CAGUACACCUUGUCACUAU | 1576-1594 | — | 1619-1637 | — | — |
| Sp | | | | | | | | |
| Cross | 19 | CAUUGUGUACGUGGACCAU | AUGGUCCACGUACACAAUG | 842-860 | — | 885-903 | — | — |
| Sp | | | | | | | | |
| Cross | 19 | CCCAGAGGACAUUGUGUAC | GUACACAAUGUCCUCUGGG | 833-851 | — | 876-894 | — | — |
| Sp | | | | | | | | |
| Cross | 19 | GCUCAUUGCCAGCUUGGAC | GUCCAAGCUGGCAAUGAGC | 2375-2393 | — | 2398-2416 | — | — |
| Sp | | | | | | | | |
| Cross | 19 | CAGGAUGAUGGCAGCUUCA | UGAAGCUGCCAUCAUCCUG | 1602-1620 | — | 1645-1663 | — | — |
| Sp | | | | | | | | |
| Cross | 19 | AAUGCGGCAGAUGACGACU | AGUCGUCAUCUGCCGCAUU | 243-261 | — | 316-334 | — | — |
| Sp | | | | | | | | |
| Cross | 19 | GAGGACAUUGUGUACGUGG | CCACGUACACAAUGUCCUC | 837-855 | — | 880-898 | — | — |
| Sp | | | | | | | | |
| Cross | 19 | CAGUGGCAUCUUCUGCUGC | GCAGCAGAAGAUGCCACUG | 1478-1496 | — | 1521-1539 | — | — |
| Sp | | | | | | | | |
| Cross | 19 | CCAGUGCUGGGUCUUUGCU | AGCAAAGACCCAGCACUGG | 1205-1223 | — | 1248-1266 | — | — |
| Sp | | | | | | | | |
| Cross | 19 | GGACAUUGUGUACGUGGAC | GUCCACGUACACAAUGUCC | 839-857 | — | 882-900 | — | — |
| Sp | | | | | | | | |
| Single | 19 | GAGAGGGCAUGCUAGUAGU | ACUACUAGCAUGCCCUCUC | 397-415 | — | 440-452 | — | — |
| Sp | | | | | | | | |
| Cross | 19 | UGUCUCAGGCCACGUCAAG | CUUGACGUGGCCUGAGACA | 2084-2102 | — | 2107-2125 | — | — |
| Sp | | | | | | | | |
| Cross | 19 | CUGCUCAAUGUCUCAGGCC | GGCCUGAGACAUUGAGCAG | 2076-2094 | — | 2099-2117 | — | — |
| Sp | | | | | | | | |
| Cross | 19 | CUCAAUGUCUCAGGCCACG | CGUGGCCUGAGACAUUGAG | 2079-2097 | — | 2102-2120 | — | — |
| Sp | | | | | | | | |
| Cross | 19 | AUGGCAGCUUCAAGAUUGU | ACAAUCUUGAAGCUGCCAU | 1609-1627 | — | 1652-1670 | — | — |
| Sp | | | | | | | | |
| Cross | 19 | CAGCGUGGAGAUCCUGCUU | AAGCAGGAUCUCCACGCUG | 1151-1169 | — | 1194-1212 | — | — |
| Sp | | | | | | | | |
| Cross | 19 | GAGGUGAAUAGUGACAAGG | CCUUGUCACUAUUCACCUC | 1569-1587 | — | 1612-1630 | — | — |
| Sp | | | | | | | | |
| Cross | 19 | AGUGGCAUCUUCUGCUGCG | CGCAGCAGAAGAUGCCACU | 1479-1497 | — | 1522-1540 | — | — |
| Sp | | | | | | | | |
| Cross | 19 | GACAUUGUGUACGUGGACC | GGUCCACGUACACAAUGUC | 840-858 | — | 883-901 | — | — |
| Sp | | | | | | | | |
| Cross | 19 | CCAGAGGACAUUGUGUACG | CGUACACAAUGUCCUCUGG | 834-852 | — | 877-895 | — | — |
| Sp | | | | | | | | |
| Cross | 19 | CCUGGAUGACAAUGGAGUC | GACUCCAUUGUCAUCCAGG | 1073-1091 | — | 1116-1134 | — | — |
| Sp | | | | | | | | |
| Cross | 19 | UGAAUAGUGACAAGGUGUA | UACACCUUGUCACUAUUCA | 1573-1591 | — | 1616-1634 | — | — |
| Sp | | | | | | | | |
| Single | 19 | CCAAGAUAGGGGCACACUA | UAGUGUGCCCCUAUCUUGG | 2585-2603 | — | — | — | — |
| Sp | | | | | | | | |
| Cross | 19 | CAUUGCCAGCUUGGACAGC | GCUGUCCAAGCUGGCAAUG | 2378-2396 | — | 2401-2419 | — | — |
| Sp | | | | | | | | |
| Cross | 19 | UCAAUGUCUCAGGCCACGU | ACGUGGCCUGAGACAUUGA | 2080-2098 | — | 2103-2121 | — | — |
| Sp | | | | | | | | |
| Cross | 19 | AUGAGUCUGGGAGAAUUUA | UAAAUUCUCCCAGACUCAU | 889-907 | — | 932-950 | — | — |
| Sp | | | | | | | | |
| Cross | 19 | GUGAAUAGUGACAAGGUGU | ACACCUUGUCACUAUUCAC | 1572-1590 | — | 1615-1633 | — | — |
| Sp | | | | | | | | |
| Cross | 19 | CAGUGCUGGGUCUUUGCUG | CAGCAAAGACCCAGCACUG | 1206-1224 | — | 1249-1267 | — | — |
| Sp | | | | | | | | |
| Cross | 19 | AGCGUGGAGAUCCUGCUUA | UAAGCAGGAUCUCCACGCU | 1152-1170 | — | 1195-1213 | — | — |
| Sp | | | | | | | | |
| Cross | 19 | UCUCAGGCCACGUCAAGGA | UCCUUGACGUGGCCUGAGA | 2086-2104 | — | 2109-2127 | — | — |
| Sp | | | | | | | | |
| Cross | 19 | GUGUACGUGGACCAUGAGG | CCUCAUGGUCCACGUACAC | 846-864 | — | 889-907 | — | — |
| Sp | | | | | | | | |
| Cross | 19 | UGCCCAGAGGACAUUGUGU | ACACAAUGUCCUCUGGGCA | 831-849 | — | 874-892 | — | — |
| Sp | | | | | | | | |
| Cross | 19 | UUGUGUACGUGGACCAUGA | UCAUGGUCCACGUACACAA | 844-862 | — | 887-905 | — | — |
| Sp | | | | | | | | |
| Cross | 19 | UCAUUGCCAGCUUGGACAG | CUGUCCAAGCUGGCAAUGA | 2377-2395 | — | 2400-2418 | — | — |
| Sp | | | | | | | | |
| Cross | 19 | CGUGGAGAUCCUGCUUAGC | GCUAAGCAGGAUCUCCACG | 1154-1172 | — | 1197-1215 | — | — |
| Sp | | | | | | | | |
| Single | 19 | GGGAACAGAUGCUAAUAAA | UUUAUUAGCAUCUGUUCCC | 2678-2696 | — | — | — | — |
| Sp | | | | | | | | |
| Cross | 19 | AUGUGGCCAUGCAGGUGGA | UCCACCUGCAUGGCCACAU | 1813-1831 | — | 1836-1854 | — | — |
| Sp | | | | | | | | |
| Single | 19 | CAAGGACAUUGCCCCAAGA | UCUUGGGGCAAUGUCCUUG | 2572-2590 | — | — | — | — |
| Sp | | | | | | | | |
| Cross | 19 | GCCCAGAGGACAUUGUGUA | UACACAAUGUCCUCUGGGC | 832-850 | — | 875-893 | — | — |
| Sp | | | | | | | | |
| Cross | 19 | UGAUGGCAGCUUCAAGAUU | AAUCUUGAAGCUGCCAUCA | 1607-1625 | — | 1650-1668 | — | — |
| Sp | | | | | | | | |
| Cross | 19 | ACAUUGUGUACGUGGACCA | UGGUCCACGUACACAAUGU | 841-859 | — | 884-902 | — | — |
| Sp | | | | | | | | |
| Cross | 19 | GCUCAAUGUCUCAGGCCAC | GUGGCCUGAGACAUUGAGC | 2078-2096 | — | 2101-2119 | — | — |
| Sp | | | | | | | | |
| Cross | 19 | AUGUCUCAGGCCACGUCAA | UUGACGUGGCCUGAGACAU | 2083-2101 | — | 2106-2124 | — | — |
| Sp | | | | | | | | |
| Cross | 19 | UAGUGACAAGGUGUACUGG | CCAGUACACCUUGUCACUA | 1577-1595 | — | 1620-1638 | — | — |
| Sp | | | | | | | | |
| Cross | 19 | CUGGAUGACAAUGGAGUCC | GGACUCCAUUGUCAUCCAG | 1074-1092 | — | 1117-1135 | — | — |
| Sp | | | | | | | | |
| Cross | 19 | GGUGCCCAGAGGACAUUGU | ACAAUGUCCUCUGGGCACC | 829-847 | — | 872-890 | — | — |
| Sp | | | | | | | | |
| Cross | 19 | GCUGCUCAAUGUCUCAGGC | GCCUGAGACAUUGAGCAGC | 2075-2093 | — | 2098-2116 | — | — |
| Sp | | | | | | | | |
| Cross | 19 | UGCUGCUCAAUGUCUCAGG | CCUGAGACAUUGAGCAGCA | 2074-2092 | — | 2097-2115 | — | — |
| Sp | | | | | | | | |
| Cross | 19 | UAUGGCCAGUGCUGGGUCU | AGACCCAGCACUGGCCAUA | 1200-1218 | — | 1243-1261 | — | — |
| Sp | | | | | | | | |
| Cross | 19 | AGGUGAAUAGUGACAAGGU | ACCUUGUCACUAUUCACCU | 1570-1588 | — | 1613-1631 | — | — |
| Sp | | | | | | | | |
| Cross | 19 | CUCAUUGCCAGCUUGGACA | UGUCCAAGCUGGCAAUGAG | 2376-2394 | — | 2399-2417 | — | — |
| Sp | | | | | | | | |
| Cross | 19 | AAUAGUGACAAGGUGUACU | AGUACACCUUGUCACUAUU | 1575-1593 | — | 1618-1636 | — | — |
| Sp | | | | | | | | |
| Cross | 19 | UGUGUACGUGGACCAUGAG | CUCAUGGUCCACGUACACA | 845-863 | — | 888-906 | — | — |
| Sp | | | | | | | | |
| Cross | 19 | GGCAGGAUGAUGGCAGCUU | AAGCUGCCAUCAUCCUGCC | 1600-1618 | — | 1643-1661 | — | — |
| Sp | | | | | | | | |
| Cross | 19 | GGGCCAUGCUGCUCAAUGU | ACAUUGAGCAGCAUGGCCC | 2068-2086 | — | 2091-2109 | — | — |
| Sp | | | | | | | | |
| Cross | 19 | GCAGGAUGAUGGCAGCUUC | GAAGCUGCCAUCAUCCUGC | 1601-1619 | — | 1644-1662 | — | — |
| Sp | | | | | | | | |
| Cross | 19 | UCAGGCCACGUCAAGGAGA | UCUCCUUGACGUGGCCUGA | 2088-2106 | — | j2111-2129 | — | — |
| Sp | | | | | | | | |
| Cross | 19 | AUGGCCAGUGCUGGGUCUU | AAGACCCAGCACUGGCCAU | 1201-1219 | — | 1244-1262 | — | — |
| Sp | | | | | | | | |
| Single | 19 | GCACCACACAGACGAGUAU | AUACUCGUCUGUGUGGUGC | 464-482 | — | 507-523 | — | — |
| Sp | | | | | | | | |
| Cross | 19 | UGGUGCCCAGAGGACAUUG | CAAUGUCCUCUGGGCACCA | 828-846 | — | 871-889 | — | — |
| Sp | | | | | | | | |
| Cross | 19 | GUGCCCAGAGGACAUUGUG | CACAAUGUCCUCUGGGCAC | 830-848 | — | 873-891 | — | — |
| Sp | | | | | | | | |
| Cross | 19 | GGCCAUGCUGCUCAAUGUC | GACAUUGAGCAGCAUGGCC | 2069-2087 | — | 2092-2110 | — | — |
| Sp | | | | | | | | |
| Cross | 19 | GAUGAUGGCAGCUUCAAGA | UCUUGAAGCUGCCAUCAUC | 1605-1623 | — | 1648-1666 | — | — |
| Sp | | | | | | | | |
| Cross | 19 | AGGAUGAUGGCAGCUUCAA | UUGAAGCUGCCAUCAUCCU | 1603-1621 | — | 1646-1664 | — | — |
| Sp | | | | | | | | |
| Cross | 19 | AGAGGACAUUGUGUACGUG | CACGUACACAAUGUCCUCU | 836-854 | — | 879-897 | — | — |
| Sp | | | | | | | | |
| Cross | 19 | GCCAUGCUGCUCAAUGUCU | AGACAUUGAGCAGCAUGGC | 2070-2088 | — | 2093-2111 | — | — |
| Sp | | | | | | | | |
| Cross | 19 | AGUGCUGGGUCUUUGCUGG | CCAGCAAAGACCCAGCACU | 1207-1225 | — | 1250-1268 | — | — |
| Sp | | | | | | | | |
| Cross | 19 | AGGAUGUGGCCAUGCAGGU | ACCUGCAUGGCCACAUCCU | 1810-1828 | — | 1833-1851 | — | — |
| Sp | | | | | | | | |
| Cross | 19 | AUCUCUGCCAUGGUGAACU | AGUUCACCAUGGCAGAGAU | 1053-1071 | — | 1096-1114 | — | 659-677 |
| Sp | | | | | | | | |
| Cross | 19 | AGCUCAUUGCCAGCUUGGA | UCCAAGCUGGCAAUGAGCU | 2374-2392 | — | 2397-2415 | — | 1980-1998 |
| Sp | | | | | | | | |
| Cross | 19 | GGUCAUCUCUGCCAUGGUG | CACCAUGGCAGAGAUGACC | 1049-1067 | — | 1092-1110 | — | 655-673 |
| Sp | | | | | | | | |
| Cross | 19 | CCAGCUCAUUGCCAGCUUG | CAAGCUGGCAAUGAGCUGG | 2372-2390 | — | 2395-2413 | — | 1978-1996 |
| Sp | | | | | | | | |
| Cross | 19 | GGGUCAUCUCUGCCAUGGU | ACCAUGGCAGAGAUGACCC | 1048-1066 | — | 1091-1109 | — | 654-672 |
| Sp | | | | | | | | |
| Cross | 19 | CAGCUCAUUGCCAGCUUGG | CCAAGCUGGCAAUGAGCUG | 2373-2391 | — | 2396-2414 | — | 1979-1997 |
| Sp | | | | | | | | |
| Cross | 19 | GUCAUCUCUGCCAUGGUGA | UCACCAUGGCAGAGAUGAC | 1050-1068 | — | 1093-1111 | — | 656-674 |
| Sp | | | | | | | | |
| Cross | 19 | CAUCUCUGCCAUGGUGAAC | GUUCACCAUGGCAGAGAUG | 1052-1070 | — | 1095-1113 | — | 658-676 |
| Sp | | | | | | | | |
| Cross | 19 | GCCAGCUCAUUGCCAGCUU | AAGCUGGCAAUGAGCUGGC | 2371-2389 | — | 2394-2412 | — | 1977-1995 |
| Sp | | | | | | | | |
| Cross | 19 | UCAUCUCUGCCAUGGUGAA | UUCACCAUOGCAGAGAUGA | 1051-1069 | — | 1094-1112 | — | 657-675 |
| Sp | | | | | | | | |
| Single | 19 | UGACCGGUGUGGAUUUGCU | AGCAAAUCCACACCGGUCA | — | — | 458-470 | — | — |
| Sp | | | | | | | | |
| Single | 19 | GCAAUGAAAUCUACAUCCU | AGGAUGUAGAUUUCAUUGC | — | — | 842-860 | — | — |
| Sp | | | | | | | | |
| Single | 19 | GCACCACACAGAUGAGUUU | AAACUCAUCUGUGUGGUGC | — | — | 507-525 | — | — |
| Sp | | | | | | | | |
| Single | 19 | GGCAGCCUUUCCACUUAGU | ACUAAGUGGAAAGGCUGCC | — | — | 554-572 | — | — |
| Sp | | | | | | | | |
| Single | 19 | GGAUGGCAAUAAACUAUUU | AAAUAGUUUAUUGCCAUCC | — | — | 2705-2723 | — | — |
| Sp | | | | | | | | |
| Single | 19 | GCAGGAUUAUGUGCUGAAU | AUUCAGCACAUAAUCCUGC | — | — | 915-933 | — | — |
| Sp | | | | | | | | |
| Single | 19 | GGAACUAUGGCCAGUUUGA | UCAAACUGGCCAUAGUUCC | — | — | 986-1004 | — | — |
| Sp | | | | | | | | |

TABLE C
TGase 1:21-mers

| | Oligo | | | human | rat | mouse | rabbit | dog | Cow | Chimpanzee |
|---|---|---|---|---|---|---|---|---|---|---|
| Source | Length | Sense sIRNA | AntiSense siRNA | 4507474 | 13928911 | 31982704 | 165769 | 50978753 | 38641347 | 55640444 |
| Single Sp | 21 | CAAGGACAUUGCCCCAAGAUA | UAUCUUGGGGCAAUGUCCUUG | 2572-2592 | 2616-2635 | 2645-2664 | — | — | — | — |
| Single Sp | 21 | GCAAUGAGAUCUACAUCCUCU | AGAGGAUGUAGAUCUCAUUGC | 799-819 | 846-866 | 875-895 | 842-862 | 23-738 | 05-425 | 722-742 |
| Single Sp | 21 | GCAGGAGUAUGUUCUUAAUGA | UCAUUAAGAACAUACUCCUGC | 872-892 | — | — | — | — | — | 795-815 |
| Single Sp | 21 | CCAUGAUUCUGUCUGGAACUU | AAGUUCCAGACAGAAUCAUGG | 1364-1384 | — | 1440-1460 | — | 1283-1303 | 970-990 | 1287-1307 |
| Single Sp | 21 | GGAGUAUGUUCUUAAUGAGUC | GACUCAUUAAGAACAUACUCC | 875-895 | 926-942 | 955-971 | — | 794-814 | 481-501 | 798-818 |
| Single Sp | 21 | CCAGGAGUGUGAAGUACAGAU | AUCUGUACUUCACACUCCUGG | 2192-2212 | — | — | — | 2111-2131 | 1798-1818 | 2049-2069 |
| Single Sp | 21 | GCAGGAUGAUGGCAGCUUCAA | UUGAAGCUGCCAUCAUCCUGC | 1601-1621 | 1648-1668 | 1677-1694 | 1644-1664 | 1521-1540 | 208-1227 | 1524-1544 |
| Cross Sp | 21 | UGAACUCCCUGGAUGACAAUG | CAUUGUCAUCCAGGGAGUUCA | 1066-1086 | 1113-1133 | 1142-1162 | 1109-1129 | 485-1005 | 572-692 | 989-1009 |
| Cross Sp | 21 | CCAGCUCAUUGCCAGCUUGGA | UCCAAGCUGGCAAUGAGCUGG | 2372-2392 | 2419-2433 | 2448-2468 | 2395-2415 | 2291-2311 | 1978-1998 | 7299-2249 |
| Cross Sp | 21 | CCAUGCUGCUCAAUGUCUCAG | CUGAGACAUUGAGCAGCAUGG | 2071-2091 | 2124-2138 | 2153-2167 | 2094-2114 | 1990-2010 | 677-1689 | 1928-1948 |
| Single Sp | 21 | CCAUCAUCGGCAAGUUUCAGU | ACUGAAACUUGCCGAUGAUGG | 724-744 | 771-789 | 800-820 | — | 543-663 | 330-350 | 647-667 |
| Single Sp | 21 | GCAUCACCCUUGAGUGACUCA | UGAGUAACUCAAGGGUGAUGC | 565-585 | — | — | — | — | — | 488-508 |
| Single Sp | 21 | GGUGCUGGAUGCCUGCUUAUA | UAUAAGCAGGCAUCCAGCACC | 968-988 | 1015-1030 | 1044-1059 | 1011-1026 | 887-898 | — | 891-911 |
| Cross Sp | 21 | GAACUCCCUGGAUGACAAUGG | CCAUUGUCAUCCAGGGAGUUC | 1067-1087 | 1114-1134 | 1143-1163 | 1110-1130 | 986-1006 | 673-693 | 990-1010 |
| Cross Sp | 21 | AUGGUGAACUCCCUGGAUGAC | GUCAUCCAGGGAGUUCACCAU | 1062-1082 | 1109-1129 | 1138-1158 | 1105-1125 | 981-1001 | 668-688 | 985-1005 |
| Cross Sp | 21 | UCUCUGCCAUGGUGAACUCCC | GGGAGUUCACCAUGGCAGAGA | 1054-1074 | 1101-1121 | 1130-1150 | 1097-1117 | 473-992 | 660-680 | 977-997 |
| Cross Sp | 21 | CCAUGGUGAACUCCCUGGAUG | CAUCCAGGGAGUUCACCAUGG | 1060-1080 | 1107-1127 | 1136-1156 | 1103-1123 | 479-999 | 666-686 | 1983-1003 |
| Cross Sp | 21 | GCGUGGAGAUCCUGCUUAGCU | AGCUAAGCAGGAUCUCCACGC | 1153-1173 | 1202-1220 | 1231-1249 | 1196-1216 | 1072-1084 | 759-771 | 1076-1096 |
| Cross Sp | 21 | UUGUGUACGUGGACCAUGAGG | CCUCAUGGUCCACGUACACAA | 844-864 | — | 922-940 | 887-907 | 765-783 | 452-470 | 767-787 |
| Cross Sp | 21 | UGGAGAUCCUGCUUAGCUACC | GGUAGCUAAGCAGGAUCUCCA | 1156-1176 | 1203-1223 | 1232-1252 | 1199-1219 | — | — | 1079-1099 |
| Cross Sp | 21 | UGCUCAAUGUCUCAGGCCACG | CGUGGCCUGAGACAUUGAGCA | 2077-2097 | 2124-2142 | 2153-2171 | 2100-2120 | 1996-2016 | 1683-1703 | 1934-1954 |
| Cross Sp | 21 | AUAGUGACAAGGUGUACUGGC | GCCAGUACACCUUGUCACUAU | 1576-1596 | — | — | 1619-1639 | — | — | 1499-1519 |
| Cross Sp | 21 | GGGCCAUGCUGCUCAAUGUCU | AGACAUUGAGCAGCAUGGCCC | 2068-2088 | 2115-2135 | 2144-2164 | 2091-2111 | 1990-2007 | 1674-1689 | 1925-1945 |
| Cross Sp | 21 | ACAUUGUGUACGUGGACCAUG | CAUGGUCCACGUACACAAUGU | 841-861 | — | 1702-1712 | 884-904 | 760-780 | — | 764-784 |
| Single Sp | 21 | CUGAUCGCAUCACCCUUGAGU | ACUCAAGGGUGAUGCGAUCAG | 559-579 | — | — | — | — | — | 482-502 |
| Cross Sp | 21 | GGUCAUCUCUGCCAUGGUGAA | UUCACCAUGGCAGAGAUGACC | 1049-1069 | 1096-1116 | 1130-1145 | 1092-1112 | 968-988 | 655-675 | 972-992 |
| Cross Sp | 21 | UCAUCUCUGCCAUGGUGAACU | AGUUCACCAUGGCAGAGAUGA | 1051-1071 | 1101-1118 | 1130-1147 | 1094-1114 | 970-990 | 657-677 | 974-994 |
| Cross Sp | 21 | CUCCCUGGAUGACAAUGGAGU | ACUCCAUUGUCAUCCAGGGAG | 1070-1090 | 1117-1137 | 1146-1166 | 1113-1133 | 989-1006 | 676-693 | 993-1013 |
| Cross Sp | 21 | GGAGAUCCUGCUUAGCUACCU | AGGUAGCUAAGCAGGAUCUCC | 1157-1177 | 1204-1224 | 1233-1253 | 1200-1220 | — | — | 1080-1100 |
| Cross Sp | 21 | UGAAUAGUGACAAGGUGUACU | AGUACACCUUGUCACUAUUCA | 1573-1593 | — | — | 1616-1636 | — | — | 1496-1516 |
| Cross Sp | 21 | UCAUUGCCAGCUUGGACAGCC | GGCUGUCCAAGCUGGCAAUGA | 2377-2397 | — | 2453-2471 | 2400-2420 | 2296-2316 | 1983-2003 | 2234-2254 |
| Cross Sp | 21 | 4GAGGACAUUGUGUACGUGGA | UCCACGUACACAAUGUCCUCU | 836-856 | — | 1702-1712 | 879-899 | 755-775 | — | 758-779 |
| Cross Sp | 21 | GCAGCGUGGAGAUCCUGCUUA | UAAGCAGGAUCUCCACGCUGC | 1150-1170 | 1197-1217 | 1226-1244 | 1193-1213 | 1069-1084 | 756-771 | 1073-1093 |
| Cross Sp | 21 | CAAUGUCUCAGGCCACGUCAA | UUGACGUGGCCUGAGACAUUG | 2081-2101 | 2128-2148 | 2157-2177 | 2104-2124 | 2000-2020 | 1692-1704 | 1938-1958 |
| Cross Sp | 21 | UAUGGCCAGUGCUGGGUCUUU | AAAGACCCAGCACUGGCCAUA | 1200-1220 | 1247-1267 | 1279-1296 | 1243-1263 | 1119-1138 | 809-825 | 1123-1143 |
| Cross Sp | 21 | CUAUAAGCACCCAGAAGGCUC | GAGCCUUCUGGGUGCUUAUAG | 1709-1729 | 1756-1776 | 1785-1805 | 1752-1772 | 1628-1648 | 1316-1335 | 1632-1652 |
| Cross Sp | 21 | GCUGCUCAAUGUCUCAGGCCA | UGGCCUGAGACAUUGAGCAGC | 2075-2095 | 2124-2142 | 2153-2171 | 2098-2118 | 1994-2014 | — | 1932-1952 |
| Cross Sp | 21 | AGGACAUUGUGUACGUGGACC | GGUCCACGUACACAAUGUCCU | 838-858 | — | 1702-1712 | 881-901 | 757-777 | — | 761-781 |
| Single Sp | 21 | GCACCACACAGACGAGUAUGA | UCAUACUCGUCUGUGUGGUGC | 464-484 | — | — | 507-523 | 383-399 | 387-407 | — |
| Cross Sp | 21 | GGUGAAUAGUGACAAGGUGUA | UACACCUUGUCACUAUUCACC | 1571-1591 | — | — | 1614-1634 | — | 1177-1188 | 1494-1514 |
| Single Sp | 21 | GAGAGGGCAUGCUAGUAGUGA | UCACUACUAGCAUGCCCUCUC | 397-417 | — | — | 440-452 | 316-328 | — | 320-340 |
| Cross Sp | 21 | GAACAUGAAGCCCCUGGAGCA | UGCUCCAGGGGCUUCAUGUUC | 1337-1357 | 1384-1404 | 1413-1424 | 1380-1400 | 1256-1276 | 943-963 | 1260-1280 |
| Cross Sp | 21 | GCAGCAAACCCAAUGUGUAUG | CAUACACAUUGGGUUUGCUGC | 1771-1791 | — | — | 1794-1812 | 1690-1708 | 1377-1397 | 1694-1714 |
| Cross Sp | 21 | UGGAGCACCUGAACCAUGAUU | AAUCAUGGUUCAGGUGCUCCA | 1351-1371 | 1398-1418 | 1435-1447 | 1396-1408 | 1270-1290 | 957-977 | 1274-1294 |
| Cross Sp | 21 | CACCCGUACUGUCACCAACUU | AAGUUGGUGACAGUACGGGUG | 1262-1282 | 1310-1329 | 1339-1358 | 1305-1325 | 1182-1201 | 868-888 | 1185-1205 |
| Cross Sp | 21 | GUACUGUCACCAACUUCAACU | AGUUGAAGUUGGUGACAGUAC | 1267-1287 | 1314-1334 | 1343-1363 | 1312-1330 | 1188-1206 | 873-893 | 1190-1210 |
| Cross Sp | 21 | UGGUCUACAUGAAGUACGACA | UGUCGUACUUCAUGUAGACCA | 1531-1551 | 1580-1598 | 1609-1627 | 1574-1594 | 1452-1470 | 1137-1157 | 1454-1474 |
| Single Sp | 21 | GGCUGCUGUUCAUGCCGAAAU | AUUUCGGCAUGAACAGCAGCC | 225-245 | — | — | — | — | — | 148-168 |
| Cross Sp | 21 | GCACCUGAACCAUGAUUCUGU | ACAGAAUCAUGGUUCAGGUGC | 1355-1375 | 1402-1422 | 1435-1451 | 1398-1418 | 1274-1294 | 961-981 | 1278-1298 |
| Cross Sp | 21 | CCAGUGGGCAGAAUCUGAACC | GGUUCAGAUUCUGCCCACUGG | 679-699 | — | — | — | 598-618 | 285-305 | 602-622 |
| Cross Sp | 21 | UACAGAUUGUCUUCAAGAACC | GGUUCUUGAAGACAAUCUGUA | 2206-2226 | — | — | — | 2125-2145 | 1812-1832 | 2063-2083 |
| Cross Sp | 21 | UAAGCACCCAGAAGGCUCAGA | UCUGAGCCUUCUGGGUGCUUA | 1712-1732 | 1759-1779 | 1788-1808 | 1755-1772 | 1635-1651 | 318-1338 | 1635-1655 |
| Cross Sp | 21 | CCCGUACUGUCACCAACUUCA | UGAAGUUGGUGACAGUACGGG | 1264-1284 | 1311-1331 | 1340-1360 | 1307-1327 | 1183-1203 | 870-890 | 1187-1207 |
| Cross Sp | 21 | AGUACAGAUUGUCUUCAAGAA | UUCUUGAAGACAAUCUGUACU | 2204-2224 | — | — | — | 2123-2143 | 1810-1830 | 2061-2081 |
| Cross Sp | 21 | AGAACAUGAAGCCCCUGGAGC | GCUCCAGGGGCUUCAUGUUCU | 1336-1356 | 1383-1403 | 1412-1424 | 1379-1391 | 1255-1275 | 942-962 | 1259-1279 |
| Cross Sp | 21 | CCUGAACCAUGAUUCUGUCUG | CAGACAGAAUCAUGGUUCAGG | 1358-1378 | — | 1435-1451 | — | 1277-1294 | 964-984 | 1281-1301 |
| Cross Sp | 21 | GGAACUACGGCCAGUUUGACC | GGUCAAACUGGCCGUAGUUCC | 943-963 | 998-1010 | — | 986-1006 | — | 549-569 | 866-886 |
| Cross Sp | 21 | GAACCAUGAUUCUGUCUGGAA | UUCCAGACAGAAUCAUGGUUC | 1361-1381 | — | 1437-1457 | — | 1280-1300 | 967-987 | 1284-1304 |
| Cross Sp | 21 | UGGUGAACUCCCUGGAUGACA | UGUCAUCCAGGGAGUUCACCA | 1063-1083 | 1110-1130 | 1139-1159 | 1106-1126 | 982-1002 | 669-689 | 986-1006 |
| Cross Sp | 21 | CUCAAUGUCUCAGGCCACGUC | GACGUGGCCUGAGACAUUGAG | 2079-2099 | 2126-2142 | 2155-2171 | 2102-2122 | 1998-2018 | 1692-1704 | 1936-1956 |
| Cross Sp | 21 | GGACAUUGUGUACGUGGACCA | UGGUCCACGUACACAAUGUCC | 839-859 | — | 1702-1712 | 882-902 | 758-778 | — | 762-782 |
| Cross Sp | 21 | UCUCAGGCCACGUCAAGGAGA | UCUCCUUGACGUGGCCUGAGA | 2086-2106 | — | — | 2109-2129 | 2005-2025 | 1692-1712 | 1943-1963 |
| Cross Sp | 21 | UGUCUCAGGCCACGUCAAGGA | UCCUUGACGUGGCCUGAGACA | 2084-2104 | 2131-2151 | 2160-2180 | 2107-2127 | 2003-2023 | 1692-1710 | 1941-1961 |
| Cross Sp | 21 | AUGUCUCAGGCCACGUCAAGG | CCUUGACGUGGCCUGAGACAU | 2083-2103 | 2130-2150 | 2159-2179 | 2106-2126 | 2002-2022 | 1692-1709 | 1940-1960 |
| Cross Sp | 21 | CUCAUUGCCAGCUUGGACAGC | GCUGUCCAAGCUGGCAAUGAG | 2376-2396 | 2423-2442 | 2452-2471 | 2399-2419 | 2295-2315 | 1982-1998 | 2233-2253 |
| Cross Sp | 21 | CAGCUCAUUGCCAGCUUGGAC | GUCCAAGCUGGCAAUGAGCUG | 2373-2393 | 2420-2433 | 2449-2469 | 2396-2416 | 2292-2312 | 1979-1998 | 2230-2250 |
| Cross Sp | 21 | CAGAGGACAUUGUGUACGUGG | CCACGUACACAAUGUCCUCUG | 835-855 | — | — | 878-898 | — | — | 758-778 |
| Cross Sp | 21 | CCCUGGAUGACAAUGGAGUCC | GGACUCCAUUGUCAUCCAGGG | 1072-1092 | 1119-1137 | 1148-1166 | 1115-1135 | 994-1011 | 678-698 | 995-1015 |
| Cross Sp | 21 | GCCCAGAGGACAUUGUGUACG | CGUACACAAUGUCCUCUGGGC | 832-852 | 881-897 | 908-926 | 875-895 | 751-771 | 438-456 | 755-775 |
| Cross Sp | 21 | CUAUGGCCAGUGCUGGGUCUU | AAGACCCAGCACUGGCCAUAG | 1199-1219 | 1246-1266 | 1279-1295 | 1242-1262 | 1118-1138 | 809-825 | 1122-1142 |
| Cross Sp | 21 | UCUAUAAGCACCCAGAAGGCU | AGCCUUCUGGGUGCUUAUAGA | 1708-1728 | 1755-1775 | 1784-1804 | 1751-1771 | 1627-1647 | 1316-1334 | 1631-1651 |
| Cross Sp | 21 | GGCCAUGCUGCUCAAUGUCUC | GAGACAUUGAGCAGCAUGGCC | 2069-2089 | 2124-2136 | 2153-2165 | 2092-2112 | 1990-2008 | 1675-1689 | 1926-1946 |
| Cross Sp | 21 | AAACAACCCCGAGGUGGGCAA | UUGCCCACCUCGGGGUUGUUU | 590-610 | 637-657 | 676-686 | 633-653 | — | 206-216 | 513-533 |
| Cross Sp | 21 | GUGCCCAGAGGACAUUGUGUA | UACACAAUGUCCUCUGGGCAC | 830-850 | 881-897 | 906-926 | 873-893 | 749-769 | 436-456 | 753-773 |
| Cross Sp | 21 | AAUGUCUCAGGCCACGUCAAG | CUUGACGUGGCCUGAGACAUU | 2082-2102 | 2129-2149 | 2158-2178 | 2105-2125 | 2001-2021 | 16924704 | 1939-1959 |
| Cross Sp | 21 | UGUGUACGUGGACCAUGAGGA | UCCUCAUGGUCCACGUACACA | 845-865 | — | 922-941 | 888-908 | 765-784 | 452-471 | 768-788 |
| Cross Sp | 21 | GUCAUCUCUGCCAUGGUGAAC | GUUCACCAUGGCAGAGAUGAC | 1050-1070 | 1101-1117 | 1130-1146 | 1093-1113 | 969-989 | 656-676 | 973-993 |
| Cross Sp | 21 | GGUGCCCAGAGGACAUUGUGU | ACACAAUGUCCUCUGGGCACC | 829-849 | 876-896 | 905-925 | 872-892 | 748-768 | 435-455 | 752-772 |
| Cross Sp | 21 | GAUGAUGGCAGCUUCAAGAUU | AAUCUUGAAGCUGCCAUCAUC | 1605-1625 | 1652-1671 | 1681-1700 | 1648-1668 | 1527-1543 | 1211-1231 | 1531-1548 |
| Cross Sp | 21 | UGAUGGCAGCUUCAAGAUUGU | ACAAUCUUGAAGCUGCCAUCA | 1607-1627 | 1658-1671 | 1683-1700 | 1650-1670 | 1527-1543 | 1217-1233 | 1531-1550 |
| Cross Sp | 21 | CAGGAUGAUGGCAGCUUCAAG | CUUGAAGCUGCCAUCAUCCUG | 1602-1622 | 1649-1669 | 1878-1694 | 1645-1665 | 1521-1541 | 1208-1228 | 1525-1545 |
| Cross Sp | 21 | AUGAUGGCAGCUUCAAGAUUG | CAAUCUUGAAGCUGCCAUCAU | 1606-1626 | 1653-1671 | 1682-1700 | 1649-1669 | 1527-1543 | 1212-1232 | 1531-1549 |
| Cross Sp | 21 | GGAUGAUGGCAGCUUCAAGAU | AUCUUGAAGCUGCCAUCAUCC | 1604-1624 | 1651-1671 | 1680-1700 | 1647-1667 | 1527-1543 | 1210-1230 | 1531-1547 |
| Cross Sp | 21 | UUGACCCCCGCAAUGAGAUCU | AGAUCUCAUUGCGGGGGUCAA | 790-810 | 837-857 | 866-886 | 833-853 | — | 396-416 | 713-733 |
| Cross Sp | 21 | CGGCAGCAAACCCAAUGUGUA | UACACAUUGGGUUUGCUGCCG | 1769-1789 | 1817-1836 | 1846-1865 | 1793-1812 | 1689-1708 | 1375-1395 | 1692-1712 |
| Cross Sp | 21 | CCCGCAAUGAGAUCUACAUCC | GGAUGUAGAUCUCAUUGCGGG | 796-816 | 843-863 | 872-892 | — | 723-735 | 402-422 | 719-739 |
| Cross Sp | 21 | AACCAUGAUUCUGUCUGGAAC | GUUCCAGACAGAAUCAUGGUU | 1362-1382 | — | 1438-1458 | — | 1281-1301 | 968-988 | 1285-1305 |
| Cross Sp | 21 | CAAUGAGAUCUACAUCCUCUU | AAGAGGAUGUAGAUCUCAUUG | 800-820 | 847-867 | 876-896 | 843-863 | 723-739 | 406-426 | 723-743 |
| Cross Sp | 21 | UGGAACUACGGCCAGUUUGAC | GUCAAACUGGCCGUAGUUCCA | 942-962 | 989-1009 | — | 985-1005 | — | 548-568 | 865-885 |
| Cross Sp | 21 | AUGUGGAGGAGAAGGCCAUCG | CGAUGGCCUUCUCCUCCACAU | 1630-1650 | — | — | 1675-1691 | 1549-1567 | 1236-1256 | 1553-1573 |
| Cross Sp | 21 | ACUACGGCCAGUU0GACCACG | CGUGGUCAAACUGGCCGUAGU | 946-966 | 998-1011 | 1030-1040 | 989-1007 | — | 552-572 | 869-889 |
| Cross Sp | 21 | UGAACCAUGAUUCUGUCUGGA | UCCAGACAGAAUCAUGGUUCA | 1360-1380 | — | 1436-1456 | — | 1279-1299 | 966-986 | 1283-1303 |
| Cross Sp | 21 | GCAAACCCAAUGUGUAUGCCA | UGGCAUACACAUUGGGUUUGC | 1774-1794 | 1821-1841 | 1850-1870 | 1797-1812 | — | 1380-1400 | 1697-1717 |
| Cross Sp | 21 | AACAUGAAGCCCCUGGAGCAC | GUGCUCCAGGGGCUUCAUGUU | 1338-1358 | 1385-1405 | 1414-1424 | 1381-1401 | 1257-1277 | 944-964 | 1261-1281 |
| Cross Sp | 21 | CUGUCACCAACUUCAACUCCG | CGGAGUUGAAGUUGGUGACAG | 1270-1290 | 1317-1335 | 1348-1364 | 1313-1331 | 1189-1207 | 876-896 | 1193-1213 |
| Cross Sp | 21 | CCCCCGCAAUGAGAUCUACAU | AUGUAGAUCUCAUUGCGGGGG | 794-814 | 841-861 | 870-890 | 837-857 | 713-733 | 400-420 | 717-737 |
| Cross Sp | 21 | CAAGCAGCACACCUUCCGUCU | AGACGGAAGGUGUGCUGCUUG | 2123-2143 | 2170-2188 | 2199-2217 | 2146-2164 | 2046-2062 | 1729-1749 | 1980-2000 |
| Cross Sp | 21 | UUUGACCCCCGCAAUGAGAUC | GAUCUCAUUGCGGGGGUCAAA | 789-809 | 836-856 | 865-885 | 832-848 | — | 395-415 | 712-732 |
| Cross Sp | 21 | UCCUGAUUGGGAACUGGUCUG | CAGACCAGUUCCCAAUCAGGA | 1090-1110 | 1139-1153 | 1168-1182 | 1133-1153 | — | 696-716 | 1013-1033 |
| Cross Sp | 21 | GGCAGCAAACCCAAUGUGUAU | AUACACAUUGGGUUUGCUGCC | 1770-1790 | 1817-1837 | 1846-1866 | 1793-1812 | 1689-1708 | 1376-1396 | 1693-1713 |
| Cross Sp | 21 | UGACCCCCGCAAUGAGAUCUA | UAGAUCUCAUUGCGGGGGUCA | 791-811 | 838-858 | 867-887 | 834-854 | — | p97-417 | 714-734 |
| Cross Sp | 21 | UGAGAUCUACAUCCUCUUCAA | UUGAAGAGGAUGUAGAUCUCA | 803-823 | 850-870 | 879-899 | 850-866 | 723-739 | 409-429 | 726-746 |
| Cross Sp | 21 | CCACCCGUACUGUCACCAACU | AGUUGGUGACAGUACGGGUGG | 1261-1281 | 1310-1328 | — | 1304-1324 | 1182-1200 | 867-887 | 1184-1204 |
| Cross Sp | 21 | GGAGCACCUGAACCAUGAUUC | GAAUCAUGGUUCAGGUGCUCC | 1352-1372 | 1399-1419 | 1435-1448 | 1396-1408 | 1271-1291 | 958-978 | 1275-1295 |
| Cross Sp | 21 | AAGUACAGAUUGUCUUCAAGA | UCUUGAAGACAAUCUGUACUU | 2203-2223 | — | — | — | 2122-2142 | 1809-1829 | 2060-2080 |
| Cross Sp | 21 | CUGGAACUACGGCCAGUUUGA | UCAAACUGGCCGUAGUUCCAG | 941-961 | 988-1008 | — | 984-1004 | — | 547-567 | 864-884 |
| Cross Sp | 21 | GAACUACGGCCAGUUUGACCA | UGGUCAAACUGGCCGUAGUUC | 944-964 | 998-1011 | 1020-1040 | 987-1007 | — | 550-570 | 867-887 |
| Cross Sp | 21 | AGCACCUGAACCAUGAUUCUG | CAGAAUCAUGGUUCAGGUGCU | 1354-1374 | 1401-1421 | 1435-1450 | 1397-1417 | 1273-1293 | 960-980 | 1277-1297 |
| Cross Sp | 21 | AUGAUUCUGUCUGGAACUUCC | GGAAGUUCCAGACAGAAUCAU | 1366-1386 | — | — | — | — | 972-992 | 1289-1309 |
| Cross Sp | 21 | CUUUGACCCCCGCAAUGAGAU | AUCUCAUUGCGGGGGUCAAAG | 788-808 | 835-855 | 2964-884 | 1831-848 | — | 394-414 | 711-731 |
| Cross Sp | 21 | AAGCAGCACACCUUCCGUCUG | CAGACGGAAGGUGUGCUGCUU | 2124-2144 | 2171-2188 | 2200-2217 | 2147-2164 | 2046-2063 | 1730-1750 | 1981-2001 |
| Cross Sp | 21 | ACAGAUUGUCUUCAAGAACCC | GGGUUCUUGAAGACAAUCUGU | 2207-2227 | 2255-2274 | 2283-2303 | — | 2126-2146 | 1813-1833 | 2064-2084 |
| Cross Sp | 21 | AUAAGCACCCAGAAGGCUCAG | CUGAGCCUUCUGGGUGCUUAU | 1711-1731 | 1758-1778 | 1787-1807 | 1754-1772 | 1630-1650 | 1317-1337 | 1634-1654 |
| Cross Sp | 21 | AUGAGAUCUACAUCCUCUUCA | UGAAGAGGAUGUAGAUCUCAU | 802-822 | 849-869 | 878-898 | 845-865 | 723-739 | 408-428 | 725-745 |
| Cross Sp | 21 | AAUGAGAUCUACAUCCUCUUC | GAAGAGGAUGUAGAUCUCAUU | 801-821 | 848-868 | 877-897 | 844-864 | 723-739 | 407-427 | 724-744 |
| Cross Sp | 21 | CGUACUGUCACCAACUUCAAC | GUUGAAGUUGGUGACAGUACG | 1266-1286 | 1313-1333 | 1342-1362 | 1312-1329 | 1188-1205 | 872-892 | 1189-1209 |
| Cross Sp | 21 | GUCCUGAUUGGGAACUGGUCU | AGACCAGUUCCCAAUCAGGAC | 1089-1109 | 1139-1153 | 1168-1182 | 1132-1152 | 1008-1028 | 695-715 | 1012-1032 |
| Cross Sp | 21 | GUACAGAUUGUCUUCAAGAAC | GUUCUUGAAGACAAUCUGUAC | 2205-2225 | — | — | — | 21242144 | 1811-1831 | 2082-2082 |
| Cross Sp | 21 | CCACGGCAGCAAACCCAAUGU | ACAUUGGGUUUGCUGCCGUGG | 1766-1786 | 1817-1833 | 1846-1862 | 1793-1809 | 1689-1702 | 1372-1392 | 1689-1709 |

TABLE D
TGase 3:19-mers

| | Oligo | | | | |
|---|---|---|---|---|---|
| Method | Length | Sense siRNA | AntiSense siRNA | human 39777600 | mouse 6678330 |
| Single Sp | 19 | CGAAGACAGUGACAGUGAA | UUCACUGUCACUGUCUUCG | [1570-1588] | [1590-1601] |
| Single Sp | 19 | GCAUCUGGCUCUCAAUCUU | AAGAUUGAGAGGCAGAUGC | [595-613] | — |
| Single Sp | 19 | GCAACAAGUUCCCUGCAAU | AUUGCAGGGAACUUGUUGC | [2065-2083] | — |
| Single Sp | 19 | GCAGAACAUCCCAUAAAGA | UCUUUAUGGGAUGUUCUGC | [1680-1698] | — |
| Single Sp | 19 | GAAGGACUCUGCCACAAUG | CAUUGUGGCAGAGUCCUUC | [1640-1658] | — |
| Single Sp | 19 | GCACGCUUGUACAUGAAGU | ACUUCAUGUACAAGCGUGC | [1618-1061 | — |
| Single Sp | 19 | CGGCACGCUUGUACAUGAA | UUCAUGUACAAGCGUGCCG | [1616-1634] | — |
| Single Sp | 19 | CAACGGCACGCUUGUACAU | AUGUACAAGCGUGCCGUUG | [161145311 | — |
| Single Sp | 19 | CCUGGACCAUCAUCUACAA | UUGUAGAUGAUGGUCCAGG | [1597-1615] | [1616-1634] |
| Single Sp | 19 | GCAAAGAAGUCAACCUGGU | ACCAGGUUGACUUCUUUGC | [1522-1540] | [1541-1552] |
| Single Sp | 19 | CUCUAGGAGUCCAGAGUAU | AUACUCUGGACUCCUAGAG | [43-61] | — |
| Single Sp | 19 | CAAUGGGUUUGGAAACAGA | UCUGUUUCCAAACCCAUUG | [1447-1485] | — |
| Single Sp | 19 | CAAUAGCAAUGAUGACAAU | AUUGUCAUCAUUGCUAUUG | [707-725] | [731-743] |
| Single Sp | 19 | GUGCCAUGAUCAAUAGCAA | UUGCUAUUGAUCAUGGCAC | [697-715] | — |
| Single Sp | 19 | GCAGAAAUGACCCCAAAUA | UAUUUGGGGUCAUUUCUGC | [661-679] | [680-698] |
| Single Sp | 19 | CUCAGCAUCUGCCUCUCAA | UUGAGAGGCAGAUGCUGAG | [591-609] | — |
| Single Sp | 19 | CUGGAACUUUGGACAGUUU | AAACUGUCCAAAGUUCCAG | [500-578] | [579-597] |
| Single Sp | 19 | AAACCGAAUUGGCAUGAUU | AAUCAUGCCAAUUCGGUUU | [539-678 | [562-5731 |
| Single Sp | 19 | GAAGAUGGCGGCAUCAUCU | AGAUGAUGCCGGCAUCUUC | [507-525] | — |
| Single Sp | 19 | CGUUCAUACUGCUUUUUAA | UUAAAAAGCAGUAUGAACG | [424-442] | — |
| Single Sp | 19 | GGACGUUCAUACUGCUUUU | AAAAGCAGUAUGAACGUCC | [421-439] | — |
| Single Sp | 19 | CAAUGGCCCUCCAGAUCUU | AAGAUCUGGAGGGCCAUUG | [360-388] | — |
| Single Sp | 19 | CCAUAGGACGGUACACAAU | AUUGUGUACCGUCCUAUGG | [355-373] | — |
| Single Sp | 19 | GCAAUACUCUGACUAUCAG | CUGAUAGUCAGAGUAUUGC | [313-331] | — |
| Single Sp | 19 | CCAGCAAUGGCAAUACUCU | AGAGUAUUGCCAUUGCUGG | [304-322] | — |
| Single Sp | 19 | GGCCAGCAAUGGCAAUACU | AGUAUUGCCAUUGCUGGCC | [302-320] | — |
| Single Sp | 19 | CGAAGGCUGUGUUUCCACU | AGUGGAAACACAGCCUUCG | [244-262] | — |
| Single Sp | 19 | CCAUGACGAAGGCUGUGUU | AACACAGCCUUCGUCAUGG | [238-258] | — |
| Single Sp | 19 | CACCCUAACUCAAAAUAAA | UUUAUUUUGAGUUAGGGUG | [2573-2591] | — |
| Single Sp | 19 | GCACUCACACCCUAACUCA | UGAGUUAGGGUGUGAGUGC | [2566-2584] | — |
| Single Sp | 19 | GGAGUUCAUUGUCUCCACA | UGUGGAGACAAUGAACUCC | [197-215] | — |
| Single Sp | 19 | CCUAUGACUUGAUCACUUU | AAAGUGAUCAAGUCAUAGG | [2308-2326] | — |
| Single Sp | 19 | CCACCCUGUCCUAUGACUU | AAGUCAUAGGACAGGGUGG | [2299-2317] | — |
| Single Sp | 19 | GGAGAGCUCACCAUGGAAU | AUUCCAUGGUGAGCUCUCC | [2163-2181] | — |
| Single Sp | 19 | GGCCUUGGCUCUAACCAAA | UUUCGUUAGAGCCAAGGCC | [174-192] | — |
| Single Sp | 19 | GUACAAACUUGGAcAAcAC | GUGUUGUCCAAGUUUGUAC | [2137-2155] | — |
| Single Sp | 19 | GCAAUCAAGGCCAUGUUGU | ACAACAUGGCCUuGAUUGc | [2079-2097] | [2099-2112] |
| Single Sp | 19 | CAAGUUCCCUGCAAUCAAG | CUUGAUUGCAGGGAACUUG | [2069-2081 | — |
| Single Sp | 19 | GACUUCUCCUGCAACAAGU | ACUUGUUGCAGGAGAAGUC | [2055-2073] | — |
| Single Sp | 19 | GGAGUCCAGAGUAUCAACU | AGUUGAUACUCUGGACUCC | [48-66] | — |
| Single SP | 19 | CAACAUGAUCCGGAUCACA | UGUGAUCCGGAUCAUGUUG | [1738-1754] | [1755-1770] |
| Single Sp | 19 | CAGACAACAUGAUCCGGAU | AUCCGGAUCAUGUUGUCUG | [1732-1750] | [1751-1769] |
| Single Sp | 19 | UGAAGUCAGACAACAUGAU | AUCAUGUUGUCUGACUUCA | [1726-1744] | [1745-1763] |
| Single Sp | 19 | AGAAGUACCUGAAGUCAGA | UCUGACUUCAGGUACUUCU | [1717-1735] | — |
| Single Sp | 19 | CGUACGCUCAGUAUGAGAA | UUCUCAUACUGAGCGUACG | [1702-7214] | [1727-1738] |
| Single Sp | 19 | GAUCUCGUACGCUCAGUAU | AUACUGAGCGUACGAGAUC | [1697-1716] | — |
| Single Sp | 19 | AGAUCUCGUACGCUCAGUA | UACUGAGCGUACGAGAUCU | [1696-1714] | — |
| Single Sp | 19 | CCAUAAAGAUCUCGUACGC | GCGUACGAGAUCUUUAUGG | [1690-1708] | — |
| Single Sp | 19 | GAACAUCCCAUAAAGAUCU | AGAUCUUUAUGGGAUGUUC | [16133-1701] | |

TABLE E
TGase 3:21-mers

| | Oligo | | | human | mouse |
|---|---|---|---|---|---|
| Method | Length | Sense siRNA | AntiSense siRNA | 39777600 | 6678330 |
| Single Sp | 21 | GCAACAAGUUCCCUGCAAUCA | UGAUUGCAGGGAACUUGUUGC | [2065-2085] | — |
| Single Sp | 21 | CGAAGACAGUGACAGUGAACA | UGUUCACUGUCACUGUCUUCG | [1570-1590] | [1590-1601] |
| Single Sp | 21 | CAAGGGUAGUGAUAGCGUAUG | CAUACGCUAUCACUACCCUUG | [998-1018] | [1024-1034] |
| Single Sp | 21 | CAACUCAGCUCAUGACACAGA | UCUGUGUCAUGAGCUGAGUUG | [928-948] | — |
| Single Sp | 21 | ACAAGCGCAUCACACAGACAA | UUGUCUGUGUGAUGCGCUUGU | [88-108] | [109-125] |
| Single Sp | 21 | GCGUGGAGAUCCUCAAAAAUU | AAUUUUUGAGGAUCUCCAcGC | [793-813] | [814-827] |
| Single Sp | 21 | GCAGCGUGGAGAUCCUCAAAA | UUUUGAGGAUCUCCACGCUGC | [790-810] | [814-8271 |
| Single Sp | 21 | UGAGUGCCAUGAUCAAUAGCA | UGCUAUUGAUCAUGGCACUCA | [694-714] | [713-725] |
| Single Sp | 21 | GGUGCUGAGUGCCAUGAUCAA | UUGAUCAUGGCACUCAGCACC | [889-709] | [708-7251 |
| Single Sp | 21 | CAGCAGAAAUGACCCCAAAUA | UAUUUGGGGUCAUUUCUGCUG | [659-679] | [880-6981] |
| Single Sp | 21 | CUACUGAUGUGGCCAGCAGAA | UUCUGCUGGCCACAUCAGUAG | [646-6661 | [667-677] |
| Single Sp | 21 | GCCUCUCAAUCUUGGAUAGGA | UCCUAUCCAAGAUUGAGAGGC | [601-821] | — |
| Single Sp | 21 | GCAUCUGCCUCUCAAUCUUGG | CCAAGAUUGAGAGGCAGAUGC | [595-815] | — |
| Single Sp | 21 | CUGGAACUUUGGACAGUUUGA | UCAAACUGUCCAAAGUUCCAG | [560-580] | [579-599] |
| Single Sp | 21 | GAAGAUGCCGGCAUCAUCUUU | AAAGAUGAUGCCGGCAUCUUC | [507-527] | — |
| Single Sp | 21 | GGAAGAUGCCGGCAUCAUCUU | AAGAUGAUGCCGGCAUCUUCC | [506-526] | — |
| Single Sp | 21 | UCAGGAAGAUGCCGGCAUCAU | AUGAUGCCGGCAUCUUCCUGA | [503-523] | — |
| Single Sp | 21 | GGACGUUCAUACUGCUUUUUA | UAAAAAGCAGUAUGAACGUCC | [421-441] | — |
| Single Sp | 21 | CUCUAGGAGUCCAGAGUAUCA | UGAUACUCUGGACUCCUAGAG | [43-63] | — |
| Single Sp | 21 | CAAUGGCCCUCCAGAUCUUCU | AGAAGAUCUGGAGGGCCAUUG | [370-390] | — |
| Single Sp | 21 | GCAAUACUCUGACUAUCAGCA | UGCUGAUAGUCAGAGUAUUGC | [313-333] | — |
| Single Sp | 21 | GGCCAGCAAUGGCAAUACUCU | AGAGUAUUGCCAUUGCUGGCC | [302-322] | — |
| Single Sp | 21 | GCACUCACACCCUAACUCAAA | UUUGAGUUAGGGUGUGAGUGC | [2566-2586] | — |
| Single Sp | 21 | AGAAGCUGGUCUAGACUGUUU | AAACAGUCUAGACCAGCUUCU | [2482-2482] | — |
| Single Sp | 21 | CGAAGGCUGUGUUUCCACUCU | AGAGUGGAAACACAGCCUUCG | [244-264] | — |
| Single Sp | 21 | CCACCCUGUCCUAUGACUUGA | UCAAGUCAUAGGACAGGGUGG | [2299-23191 | — |
| Single Sp | 21 | GGAGAGCUCACCAUGGAAUGA | UCAUUCCAUGGUGAGCUCUCC | [2163-2183] | — |
| Single Sp | 21 | CCAUCGAUGUAGCCGAAUGAA | UUCAUUCGGCUACAUCGAUGG | [2098-2118] | — |
| Single Sp | 21 | GCCAUGUUGUCCAUCGAUGUA | UACAUCGAUGGACAACAUGGC | [2088-2108] | — |
| Single Sp | 21 | CAACAAGUUCCCUGCAAUCAA | UUGAUUGCAGGGAACUUGUUG | [2066-2086] | — |

TABLE F
TGase 5:19-mers

| | Oligo | | | human | human | rat | mouse |
|---|---|---|---|---|---|---|---|
| Method | Length | Sense siRNA | AntiSense siRNA | 42518071 | 4759229 | 34856752 | 27229193 |
| Single Sp | 19 | CAACAGCAAUGAUGAUAAU | AUUAUCAUCAUUGCUGUUG | [695-713] | [449-467] | [751-764] | [791-804] |
| Single Sp | 19 | GCAGGAGUAUGUCAUGAAU | AUUCAUGACAUACUCCUGC | [479-497] | [233-251] | [534-545] | [574-585] |
| Single Sp | 19 | GAAGUGGCCUCUUCAAGAA | UUCUUGAAGAGGCCACUUC | [1984-2002] | [1738-1756] | — | — |
| Single Sp | 19 | GAACUUCCAUGUCUGGAAU | AUUCCAGACAUGGAAGUUC | [1010-1028] | [764-782] | — | — |
| Single Sp | 19 | UGAGUAUUAUGACAACACA | UGUGUUGUCAUAAUACUCA | [953-971] | [707-725] | [1008-1026] | — |
| Single Sp | 19 | GAAGCCUGAUCAUAGAUGA | UCAUCUAUGAUCAGGUUUC | [937-955] | [691-709] | [994-1010] | [1034-1050] |
| Single Sp | 19 | CGAUACAGAUGGAAACCUG | CAGGUUUCCAUCUGUAUCG | [928-944] | [680-698] | — | — |
| Single Sp | 19 | CUACCCGUGUGAUCACCAA | UUGGUGAUCACACCGGUAG | [892-910] | [646-664] | [949-965] | [989-1005] |
| Single Sp | 19 | CCAUGUCCCUGGAACUAUG | CAUAGUUCCAGGGACAUGG | [540-558] | [294-312] | [601-613] | [635-653] |
| Single Sp | 19 | AAAGGCAGAUCCAAGCUAA | UUAGCUUGGAUCUGCCUUU | [2086-2104] | [1840-1858] | [2138-2156] | [2178-2196] |
| Single Sp | 19 | GCAUCAUUCUGGAGACCGU | ACGGUCUCCAGAAUGAUGC | [2050-2068] | [1804-1822] | — | — |
| Single Sp | 19 | CCAUACAGGUGAUAUUUUC | GAAAAUAUCACCUGUAUGG | [1918-1936] | [1672-1690] | — | — |
| Single Sp | 19 | AGAAAAUCCUGGUGAACAA | UUGUUCACCAGGAUUUUCU | [1828-1846] | [1582-1600] | [1880-1898] | [1920-1938] |
| Single Sp | 19 | GAAAAGCAGUCCUGAGAAA | UUUCUCAGGACUGCUUUUC | [1814-1832] | [1568-1586] | [1870-1884] | [1912-1924] |
| Single Sp | 19 | UCAUCACACUCUCUCCUAA | UUAGGAGAGAGUGUGAUGA | [1699-1717] | [1453-1471] | — | — |
| Single Sp | 19 | UCAAGGACCUCAAAGUGAA | UUCACUUUGAGGUCCUUGA | [1615-1633] | [1369-1387] | — | — |
| Single Sp | 19 | CCAGUGAUGUGGUGCAAGU | ACUUGCACCACAUCACUGG | [1510-1528] | [1264-1282] | — | — |
| Single Sp | 19 | AGAGGCAGGUGUUUCUGAA | UUCAGAAACACCUGCCUCU | [1366-1384] | [1120-1138] | [1421-1436] | — |
| Single Sp | 19 | CUACAAGUAUGAAGAAGGA | UCCUUCUUCAUACUUGUAG | [1337-1355] | [1091-1109] | [1392-1409] | [1432-1449] |
| Single Sp | 19 | AGAUCACUGUGGACCACCU | AGGUGGUCCACAGUGAUCU | 10 [82-100] | [82-100] | — | — |
| Single Sp | 19 | UCAUCUUCGUGGUUGAAAC | GUUUCAACCACGAAGAUGA | [178-196] | [178-196] | — | — |
| Single Sp | 19 | ACAUCAUCUUCGUGGUUGA | UCAACCACGAAGAUGAUGU | [175-193] | [175-193] | — | — |
| Single Sp | 19 | ACAACAUCAUCUUCGUGGU | ACCACGAAGAUGAUGUUGU | [172-190] | [172-190] | — | — |
| Single Sp | 19 | UCACAGACCUCCAGAGCUC | GAGCUCUGGAGGUCUGUGA | [34-52] | [34-52] | [113-131] | [129-147] |
| Single Sp | 19 | GGAUUUUGGGGAAUAAGAA | UUCUUAUUCCCCAAAAUCC | [976-994] | [730-748] | — | — |
| Single Sp | 19 | GCAGGAUUUUGGGGAAUAA | UUAUUCCCCAAAAUCCUGC | [973-991] | [727-745] | — | — |
| Single Sp | 19 | CCCGUGUGAUCACCAACUU | AAGUUGGUGAUCACACGGG | [895-913] | [649-667] | [950-968] | [990-1008] |
| Single Sp | 19 | GCAAUGCUGGGUCUUUGCU | AGCAAAGACCCAGCAUUGC | [836-854] | [590-608] | — | [931-949] |
| Single Sp | 19 | GCCUGAAGCUGCUAGACAA | UUGUCUAGCAGCUUCAGGC | [589-607] | [343-361] | — | — |
| Single Sp | 19 | CUGGAACUAUGGACAGUUU | AAACUGUCCAUAGUUCCAG | [548-566] | [302-320] | [603-621] | [643-661] |
| Cross Sp | 19 | CUCCAGAGCUCCAGAAAUA | UAUUUCUGGAGCUCUGGAG | [42-60] | [42-60] | [121-132] | [137-148] |
| Single Sp | 19 | GGGAGUUCAUCCUGCUUUU | AAAAGCAGGAUGAACUCCC | [409-427] | [464-479] | — | — |
| Single Sp | 19 | GGUACCUCUUGAAAAUCCA | UGGAUUUUCAAGAGGUACC | [349-367] | — | — | — |
| Single Sp | 19 | GUCGGUACCUCUUGAAAAU | AUUUUCAAGAGGUACCGAC | [346-384] | — | — | — |
| Single Sp | 19 | GAAGCAACAAGUUUAAGGA | UCCUUAAACUUGUUGCUUC | [2110-2128] | [1864-1882] | — | — |
| Single Sp | 19 | GUGGCCUCUUCAAGAAAcA | UGUUUCUUGAAGAGGCCAC | [1987-2005] | [1741-1759] | — | — |
| Single Sp | 19 | GAAGGAAGUGGCCUCUUCA | UGAAGAGGCCACUUCCUUC | [1980-1998] | [1734-1752] | [2032-2048] | [2072-2088] |
| Single Sp | 19 | GAACCAGCCACUCUCCAUA | UAUGGAGAGUGGCUGGUUC | [1904-1922] | [1658-1676] | — | — |
| Single Sp | 19 | GCAUCACGAUUAAUGUUCU | AGAACAUUAAUCGUGAUGC | [1870-1888] | [1824-1642] | — | [1962-1980] |
| Single Sp | 19 | CAAGCAUCACGAUUAAUGU | ACAUUAAUCGUGAUGCUUG | [1867-1885] | [1621-1639] | — | — |
| Single Sp | 19 | GGAGUCCUGAGAAAAUCCU | AGGAUUUUCUCAGGACUGC | [1819-1837] | [1573-1591] | [1871-1889] | [1912-1929] |
| Single Sp | 19 | CAAACUGAUCCGCAUCAGU | ACUGAUGCGGAUCAGCUUG | [1781-1799] | [1535-1553] | — | [1873-1891] |
| Single Sp | 19 | GGAAGUACCUGUCAACAGA | UCUGUUGACAGGUACUGGC | [1762-17801 | [1516-1534] | [1814-1832] | — |
| Single Sp | 19 | CACACUCUCUCCUAAAGAA | UUCUUUAGGAGAGAGUGUG | [1703-1721] | [1457-1475] | — | — |
| Single Sp | 19 | CCAGUUCAAGGACCUCAAA | UUUGAGGUCCUUGAACUGG | [1610-1528] | [1364-1382] | [1663-1680] | [1703-1720] |
| Single Sp | 19 | CCAACAUGGGCCAGGAUAU | AUAUCCUGGCCCAUGUUGG | [1558-1576] | [1312-1330] | — | — |
| Single Sp | 19 | GUGCAAGUCUCCCUGAAAU | AUUUCAGGGAGACUUGCAC | [1521-1539] | [1275-1293] | — | — |
| Single Sp | 19 | GGAGCAGAGUUGCAACCUU | AAGGUUGCAACUCUGCUcc | [1431-1449] | [1185-1203] | — | — |
| Single Sp | 19 | GGGAUGACAUCACAGAGAA | UUCUCUGUGAUGUCAUCCC | [1318-1338] | [1072-1090] | — | [1415-14301 |
| Single Sp | 19 | GCAAUUUUAUCAGCACAAA | UUUGUGCUGAUAAAAUUGC | [1279-12971 | [1033-1051] | [1336-1349] | [1376-13891 |
| Single Sp | 19 | GAGUUCUGUUGGCAAUUUU | AAAAUUGCCAACAGAACUC | [1268-1286] | [1022-1040] | — | — |
| Single Sp | 19 | CGCCCUUUGUGUUUUCGAU | AUCGAAAACACAAAGGGCG | [1183-1201] | [937-9551 | — | — |
| Single Sp | 19 | GAAGGAUACUAUCUGGAAC | GUUCCAGAUAGUAUCCUUC | [995-1013] | [749-787] | — | — |
| Single Sp | 19 | GAAGAAGGAUACUAUCUGG | CCAGAUAGUAUCCUUCUUC | [992-1010] | [748-764] | [1047-1065] | — |
| Single Sp | 19 | GUAAUAAGAAGAAGGAUAC | GUAUCCUUCUUCUUAUUCC | [985-1003] | [739-757] | [1041-1058] | — |
| Single Sp | 19 | UCAACCUCACCCUGUACUU | AAGUACAGGGUGAGGUUGA | [124-142] | [124-142] | [209-221] | [225-237] |
| Single Sp | 19 | CCUUCAACCUCACCCUGUA | UACAGGGUGAGGUUGAAGG | [121-1391 | [121-139] | — | — |
| Single Sp | 19 | ACACGGAGGAGAUCACUGU | ACAGUGAUCUCCUCCGUGU | [73-91] | [73-91] | — | — |
| Single Sp | 19 | GAAAUAAUGUGCGGCACQA | UGGUGCCGCACAUUAUUUC | [55-73] | [55-73] | — | — |
| Single Sp | 19 | UGGACAACAUCAUCUUCGU | ACGAAGAUGAUGUUGUCCA | [169-187] | [169-187] | — | — |
| Single Sp | 19 | UCACCCUGUACUUCAGGAA | UUCCUGAAGUACAGGGUGA | [130-148] | [130-148] | [209-221] | [225-237] |
| Single Sp | 19 | ACCUCACCCUGUACUUCAG | CUGAAGUACAGGGUGAGGU | [127-145] | [127-145] | [209-221] | [225-237] |
| Single Sp | 19 | AUAAUGUGCGGCACCACAC | GUGUGGUGCCGCACAUUAU | [58-76] | [58-76] | [139-155] | [153-171] |
| Single Sp | 19 | CGGAGGAGAUCACUGUGGA | UCCACAGUGAUCUCCUCCG | [76-94] | [78-94] | — | — |
| Single Sp | 19 | CUAGAAGUGGCCCUCACAG | CUGUGAGGGCCACUUCUAG | [21-39] | [21-39] | [100-118] | [116-134] |
| Single Sp | 19 | AGCCAGGCCUGGACAACAU | AUGUUGUCCAGGCCUGGCU | [160-178] | [180-178] | — | — |
| Single Sp | 19 | UGGACCACCUGCUUGUUCG | CGAACAAGCAGGUGGUCCA | [91-109] | [91-109] | — | — |
| Single Sp | 19 | UCACUGUGGACCACCUGCU | AGCAGGUGGUCCACAGUGA | [85-103] | [85-103] | — | — |
| Single Sp | 19 | GGAGAUCACUGUGGACCAC | GUGGUCCACAGUGAUCUCC | [80-98] | [80-98] | — | — |
| Cross Sp | 19 | AAGUGGCCCUCACAGACCU | AGGUCUGUGAGGGCCACUU | [25-43] | [25-43] | [106-122] | [122-138] |
| Cross Sp | 19 | AGAAGUGGCCCUCACAGAC | GUCUGUGAGGGCCACUUCU | [23-41] | [23-41] | [108-120] | [122-136] |
| Single Sp | 19 | UAAUGUGCGGCACCACACG | CGUGUGGUGCCGCACAUUA | [59-77] | [59-77] | [139-156] | [154-172] |
| Single Sp | 19 | GACAACACAGGCAGGAUUU | AAAUCCUGCCUGUGUUGUC | [963-981] | [717-735] | [1018-1038] | [1058-1075] |
| Single Sp | 19 | CCAGAGCUCCAGAAAUAAU | AUUAUUUCUGGAGCUCUGG | [44-62] | [44-62] | — | — |
| Single Sp | 19 | CAUCUUCGUGGUUGAAACU | AGUUUCAACCACGAAGAUG | [179-197] | [179-197] | — | — |
| Single Sp | 19 | GCCUGGACAACAUCAUCUU | AAGAUGAUGUUGUCCAGGC | [166-184] | [168-184] | — | — |
| Single Sp | 19 | CAGGCCUGGACAACAUCAU | AUGAUGUUGUCCAGGCCUG | [163-1811 | [163-181] | — | — |
| Single Sp | 19 | CCAGAAAUAAUGUGCGGCA | UGCCGCACAUUAUUUCUGG | [52-70] | [52-70] | — | — |
| Single Sp | 19 | AGAGCUCCAGAAAUAAUGU | ACAUUAUUUCUGGAGCUCU | [48-64] | [48-64] | — | — |
| Single Sp | 19 | CAGAGCUCCAGAAAUAAUG | CAUUAUUUCUGGAGCUCUG | [45-63] | [45-63] | — | — |
| Single Sp | 19 | ACCUCCAGAGCUCCAGAAA | UUCUGGAGCUCUGGAGGU | [40-68] | [40-581 | [119-132] | [135-148] |
| Single Sp | 19 | CAACAUCAUCUUCGUGGUU | AACCACGAAGAUGAUGUUG | [173-191] | [173-191] | — | — |
| Single Sp | 19 | GGAGAACAUCAUCUUCGUG | CACGAAGAUGAUGUUGUCC | [170-188] | [170-188] | — | — |
| Single Sp | 19 | CUGUACUUCAGGAACCGGA | UCCGGUUCCUGAAGUACAG | [135-153] | [135-153] | — | — |
| Single Sp | 19 | CUCACCCUGUACUUCAGGA | UCCUGAAGUACAGGGUGAG | [129-147] | [129-147] | [209-221] | [225-237] |
| Single Sp | 19 | CAACCUCACCCUGUACUUC | GAAGUACAGGGUGAGGUUG | [125-143] | [125-143] | [209-221] | [225-237] |
| Single Sp | 19 | UUCAACCUCACCCUGUACU | AGUACAGGGUGAGGUUGAA | [123-141] | [123-141] | [209-220] | [225-236] |
| Single Sp | 19 | CAGAAAUAAUGUGCGGCAC | GUGCCGCACAUUAUUUCUG | [53-71] | [53-71] | — | — |
| Single Sp | 19 | CUCCAGAAAUAAUGUGCGG | CCGCACAUUAUUUCUGGAG | [50-68] | [50-68] | — | — |
| Single Sp | 19 | UAGAAGUGGCCCUCACAGA | UCUGUGAGGGCCACUUCUA | [22-40] | [22-40] | [101-119] | [117-135] |
| Single Sp | 19 | AAUAAUGUGCGGCACCACA | UGUGGUGCCGCACAUUAUU | [57-75] | [57-75] | — | — |
| Single Sp | 19 | AGAAAUAAUGUGCGGCACC | GGUGCCGCACAUUAUUUCU | [54-72] | [54-72] | — | — |
| Single Sp | 19 | UGUACUUCAGGAACCGGAG | CUCCGGUUCCUGAAGUACA | [136-154] | [136-154] | — | — |
| Single Sp | 19 | ACCCUGUACUUCAGGAACC | GGUUCCUGAAGUACAGGGU | [132-150] | [132-150] | [211-221] | [227-237] |
| Single Sp | 19 | AAAUAAUGUGCGGCACCAC | GUGGUGCCGCACAUUAUUU | [56-74] | [56-74] | — | — |
| Single Sp | 19 | CCACACGGAGGAGAUCACU | AGUGAUCUCCUCCGUGUGG | [71-89] | [71-89] | — | — |
| Single Sp | 19 | CCAGGCCUGGACAACAUCA | UGAUGUUGUCCAGGCCUGG | [162-180] | [162-180] | — | — |
| Single Sp | 19 | UGGCCCAAGGGCUAGAAGU | ACUUCUAGCCCUUGGGCCA | [10-28] | [10-28] | — | — |
| Single Sp | 19 | CUGUGGACCACCUGCUUGU | ACAAGCAGGUGGUCCACAG | [86-106] | [88-106] | — | — |
| Single Sp | 19 | CACUGUGGACCACCUGCUU | AAGCAGGUGGUCCACAGUG | [86-104] | [86-104] | — | — |
| Single Sp | 19 | GACCUCCAGAGCUCCAGAA | UUCUGGAGCUCUGGAGGUC | [39-871 | [39-57] | [118-132] | [134-148] |
| Single Sp | 19 | GCUAGAAGUGGCCCUCACA | UGUGAGGGCCACUUCUAGC | [20-38] | [20-38] | [199-117] | [116-133] |
| Single Sp | 19 | GAUCACUGUGGACCACCUG | CAGGUGGUCCACAGUGAUC | [83-101] | [83-101] | — | — |
| Single Sp | 19 | AUGGCCCAAGGGCUAGAAG | CUUCUAGCCCUUGGGCCAU | [9-27] | [9-27] | — | [89-99] |
| Single Sp | 19 | GGAGGAGAUCACUGUGGAC | GUCCACAGUGAUCUCCUCC | [77-95] | [77-96] | — | — |
| Single Sp | 19 | ACCACACGGAGGAGAUCAC | GUGAUCUCCUCCGUGUGGU | [70-88] | [70-88] | — | — |
| Single Sp | 19 | UACCAUGGCCCAAGGGCUA | UAGCCCUUGGGCCAUGGUA | [5-23] | [5-23] | — | [86-99] |
| Single Sp | 19 | AGACCUCCAGAGCUCCAGA | UCUGGAGCUCUGGAGGUCU | [38-56] | [38-56] | [117-1321 | [133-148] |
| Single Sp | 19 | ACAGACCUCCAGAGCUCCA | UGGAGCUCUGGAGGUCUGU | [36-54] | [36-54] | [115-132] | [131-148] |
| Single Sp | 19 | UCAGGAACCGGAGCUUCCA | UGGAAGCUCCGGUUCCUGA | [142-160] | [142-160] | — | — |
| Single Sp | 19 | GUACUUCAGGAACCGGAGC | GCUCCGGUUCCUGAAGUAC | [137-155] | [137-155] | — | — |
| Single Sp | 19 | CCCUGUACUUCAGGAACCG | CGGUUCCUGAAGUACAGGG | [133-151] | [133-161] | — | — |
| Single Sp | 19 | AGGCCUUCAACCUUCACCU | AGGGUGAGGUUGAAGGCCU | [118-136] | [118-136] | — | — |
| Single Sp | 19 | UUCAGGAACCGGAGCUUCC | GGAAGCUCCGGUUCCUGAA | [141-159] | [141-159] | — | — |

TABLE G
TGase 5:21-mers

| | Oligo | | | human | human | rat | mouse |
|---|---|---|---|---|---|---|---|
| Source | Length | Sense siRNA | AntiSense siRNA | 42518071 | 4759229 | 34856752 | 27229193 |
| Single | 21 | CCAUGUCCCUGGAACUAUGGA | UCCAUAGUUCCAGGGACAUGG | [540-560] | [294-314] | [601-614] | [635-654] |
| Sp | | | | | | | |
| Single | 21 | GCAGGAGUAUGUCAUGAAUGA | UCAUUCAUGACAUACUCCUGC | [479-499] | [233-253] | [534-545] | [574-585] |
| Sp | | | | | | | |
| Single | 21 | GGAAGGAAGUGGCCUCUUCAA | UUGAAGAGGCCACUUCCUUCC | [1979-1999] | [1733-1753] | [2031-2048] | [2071-2088] |
| Sp | | | | | | | |
| Single | 21 | CCAUACAGGUGAUAUUUUCAA | UUGAAAAUAUCACCUGUAUGG | [1918-1938] | [1733-1692] | — | — |
| Sp | | | | | | | |
| Single | 21 | CCAGUGAUGUGGUGCAAGUCU | AGACUUGCACCACAUCACUGG | [1510-1530] | [1264-1284] | — | — |
| Sp | | | | | | | |
| Single | 21 | GAACUUCCAUGUCUGGAAUGA | UCAUUCCAGACAUGGAAGUUC | [1010-1030] | [764-784] | — | — |
| Sp | | | | | | | |
| Single | 21 | GAAACCUGAUCAUAGAUGAGU | ACUCAUCUAUGAUCAGGUUUC | [937-957] | [691-711] | [994-1012] | [1034-1052] |
| Sp | | | | | | | |
| Single | 21 | CGAUACAGAUGGAAACCUGAU | AUCAGGUUUCCAUCUGUAUCG | [928-946] | [680-700] | — | — |
| Sp | | | | | | | |
| Single | 21 | CUACCCGUGUGAUCACCAACU | AGUUGGUGAUCACACGGGUAG | [892-912] | [646-666] | [949-967] | [989-1007] |
| Sp | | | | | | | |
| Single | 21 | CAACAGCAAUGAUGAUAAUGG | CCAUUAUCAUCAUUGCUGUUG | [695-715] | [449-469] | [751-764] | [791-804] |
| Sp | | | | | | | |
| Single | 21 | GAAUGAUUAUGGCUUCAUCUA | UAGAUGAAGCCAUAAUCAUUC | [494-514] | [248-2681] | [553-569] | [593-609] |
| Sp | | | | | | | |
| Single | 21 | AAAGGCAGAUCCAAGCUAAUA | UAUUAGCUUGGAUCUGCCUUU | [2086-2108] | [1840-1860] | [2138-2156] | [2178-2197] |
| Sp | | | | | | | |
| Single | 21 | GAAGUGGCCUCUUCAAGAAAC | GUUUCUUGAAGAGGCCACUUC | [1984-2004] | ]1738-1758] | — | — |
| Sp | | | | | | | |
| Single | 21 | ACAAGAUCAUCACCUUAUCUU | AAGAUAAGGUGAUGAUCUUGU | [1843-1863] | [1597-1617] | [1895-1910] | [1935-1950] |
| Sp | | | | | | | |
| Single | 21 | UGAACAAGAUCAUCACCUUAU | AUAAGGUGAUGAUCUUGUUCA | [1840-1860] | [1594-1614] | [1892-1910] | [1932-1950] |
| Sp | | | | | | | |
| Single | 21 | AGAAAAUCCUGGUGAACAAGA | UCUUGUUCACCAGGAUUUUCU | [1828-1848] | [1582-1602] | [1880-1900] | [1920-1940] |
| Sp | | | | | | | |
| Single | 21 | GAAAAGCAGUCCUGAGAAAAU | AUUUUCUCAGGACUGCUUUUC | [1814-1834] | [1568-1588] | [1870-1886] | [1912-1926] |
| Sp | | | | | | | |
| Cross | 21 | ACAUCAUCUUCGUGGUUGAAA | UUUCAACCACGAAGAUGAUGU | [175-195] | [175-195] | — | — |
| Sp | | | | | | | |
| Single | 21 | CUACAAGUAUGAAGAAGGAUC | GAUCCUUCUUCAUACUUGUAG | [1337-1357] | [1091-1111] | [1392-1409] | [1432-1449] |
| Sp | | | | | | | |
| Single | 21 | AUAAUGGGGUGCUCAAUGGAA | UUCCAUUGAGCACCCCAUUAU | [709-729] | [463-483] | [772-782] | [812-822] |
| Sp | | | | | | | |
| Single | 21 | UCAAGAGCCUGCACUUCCAGA | UCUGGAAGUGCAGGCUCUUGU | [604-624] | [358-378] | — | — |
| Sp | | | | | | | |
| Single | 21 | ACAUCUGCCUGAAGCUGCUAG | CUAGCAGCUUCAGGCAGAUGU | [583-603] | [1337-357] | — | — |
| Sp | | | | | | | |
| Single | 21 | AUAGACAUCUGCCUGAAGCUG | CAGCUUCAGGCAGAUGUCUAU | [579-599] | [333-353] | [634-654] | — |
| Sp | | | | | | | |
| Single | 21 | AGAGGAUGCUGUCUACUUGGA | UCCAAGUAGACAGCAUCCUCU | [443-463] | [198-217] | — | — |
| Sp | | | | | | | |
| Single | 21 | GAAUGUUUAUGUAGACUUUGC | GCAAAGUCUACAUAAACAUUC | [2144-2164] | [1898-1918] | — | — |
| Sp | | | | | | | |
| Single | 21 | AAAUCCUGGUGAACAAGAUCA | UGAUCUUGUUCACCAGGAUUU | [1831-1851] | [1585-1605] | [1883-1903] | [1923-1943] |
| Sp | | | | | | | |
| Cross | 21 | UCAUCUUCGUGGUUGAAACUG | CAGUUUCAACCACGAAGAUGA | [178-198] | 1[78-198] | — | — |
| Sp | | | | | | | |
| Cross | 21 | ACAACAUCAUCUUCGUGGUUG | CAACCACGAAGAUGAUGUUGU | [172-192] | [172-192] | — | — |
| Sp | | | | | | | |
| Single | 21 | GCAUCACACUCUCUCCUAAAG | CUUUAGGAGAGAGUGUGAUGA | [1699-1719] | [1453-1473] | — | — |
| Sp | | | | | | | |
| Single | 21 | ACACAGCGUUCAUCACACUCU | AGAGUGUGAUGAACGCUGUGU | [1690-1710] | [1444-1464] | [1750-1760] | [1782-1800] |
| Sp | | | | | | | |
| Single | 21 | UCAAGGACCUCAAAGUGAACC | GGUUCACUUUGAGGUCCUUGA | [1615-1635] | [1369-1389] | — | — |
| Sp | | | | | | | |
| Single | 21 | GGAAGGCUCUGCAGAAGCUGA | UCAGCUUCUGCAGAGCCUUCA | [1381-1401] | [1135-1155] | [1141-1454] | [1478-1494] |
| Sp | | | | | | | |
| Single | 21 | AGAGGCAGGUGUUUCUGAAGG | CCUUCAGAAACACCUGCCUCU | [1366-1386] | [1120-1140] | [1421- 1436] | — |
| Sp | | | | | | | |
| Single | 21 | UCAGAGCCAUCAAAGAAGGAG | CUCCUUCUUUGAUGGCUCUGA | [1141-1161] | [895-915] | [1200-1216] | [1240-1256] |
| Sp | | | | | | | |
| Cross | 21 | UCACAGACCUCCAGAGCUCCA | UGGAGCUCUGGAGGUCUGUGA | [34-54] | [34-54] | [113-132] | [129-148] |
| Sp | | | | | | | |
| Single | 21 | GAAGGAUACUAUCUGGAACUU | AAGUUCCAGAUAGUAUCCUUC | [995-1015] | [749-769] | — | — |
| Sp | | | | | | | |
| Single | 21 | GGAAUAAGAAGAAGGAUACUA | UAGUAUCCUUCUUCUUAUUCC | [985-1005] | [739-759] | [1041-1059] | — |
| Sp | | | | | | | |
| Single | 21 | GGAUUUUGGGGAAUAAGAAGA | UCUUCUUAUUCCCCAAAAUCC | υ976-996] | [730-750] | — | — |
| Sp | | | | | | | |
| Single | 21 | GCAGGAUUUUGGGGAAUAAGA | UCUUAUUCCCCAAAAUCCUGC | [973-993] | [727-747] | — | — |
| Sp | | | | | | | |
| Single | 21 | CCACGAUACAGAUGGAAACCU | AGGUUUCCAUCUGUAUCGUGG | [923-943] | [677-697] | — | — |
| Sp | | | | | | | |
| Single | 21 | GCCUGAAGCUGCUAGACAAGA | UCUUGUCUAGCAGCUUCAGGC | [589-609] | [343-363] | — | — |
| Sp | | | | | | | |
| Single | 21 | CUGGAACUAUGGACAGUUUGA | UCAAACUGUCCAUAGUUCCAG | [548-568] | [302-322] | [603-623] | [643-663] |
| Sp | | | | | | | |
| Single | 21 | CCCAGAGGAUGCUGUCUACUU | AAGUAGACAGCAUCCUCUGGG | [440-460] | [198-214] | — | — |
| Sp | | | | | | | |
| Cross | 21 | CUCCAGAGCUCCAGAAAUAAU | AUUAUUUCUGGAGCUCUGGAG | [42-62] | [42-62] | [121-132] | [137-148] |
| Sp | | | | | | | |
| Single | 21 | GGGAGUUCAUCCUGCUUUUCA | UGAAAAGCAGGAUGAACUCCC | [409-429] | — | [464-484] | — |
| Sp | | | | | | | |
| Single | 21 | GCAACAAGUUUAAGGACAUUA | UAAUGUCCUUAAACUUGUUGC | [2113-2133 | [1886-1887] | — | — |
| Sp | | | | | | | |
| Single | 21 | CAACACCAAGCAAGCAUCAUU | AAUGAUGCUUGCUUGGUGUUG | [2037-2057] | [1791-1811] | — | — |
| Sp | | | | | | | |
| Single | 21 | CCAACACCAAGCAAGCAUCAU | AUGAUGCUUGCUUGGUGUUGG | [2036-2056] | [1790-1810] | — | — |
| Sp | | | | | | | |
| Single | 21 | GAACCAGCCACUCUCCAUACA | UGUAUGGAGAGUGGCUGGUUC | [1904-1924] | [1658-1678] | — | — |
| Sp | | | | | | | |
| Single | 21 | CCAAGCAUCACGAUUAAUGUU | UACAUUAAUCGUGAUGCUUGG | [1866-1886] | [1620-1640] | — | — |
| Sp | | | | | | | |
| Single | 21 | CUGAGAAAAUCCUGGUGAACA | UGUUCACCAGGAUUUUCUCAG | [1825-1845] | [1579-1599] | [1877-1897] | [1917-1937] |
| Sp | | | | | | | |
| Single | 21 | GCAAAGACCUACCCCUGCAAA | UUUGCAGGGGUAGGUCUUUGC | [1722-1742] | [1476-1496] | [776-794] | — |
| Sp | | | | | | | |
| Single | 21 | CCAGUUCAAGGACCUCAAAGU | ACUUUGAGGUCCUUGAACUGG | [1610-1630] | [1364-1384] | [1663-1680] | [1703-1720] |
| Sp | | | | | | | |
| Single | 21 | CCAACAUGGGCCAGGAUAUAU | AUAUAUCCUGGCCCAUGUUGG | [1558-1578] | [1312-1332] | — | — |
| Sp | | | | | | | |
| Single | 21 | GAAGCUGAAGGCUAGAAGCUU | AAGCUUCUAGCCUUCAGCUUC | [1394-1414] | [1148-1168] | — | — |
| Sp | | | | | | | |
| Single | 21 | GGGAUGACAUCACAGAGAACU | AGUUCUCUGUGAUGUCAUCCC | [1318-1338] | [1072-1092] | — | [1415-1430] |
| Sp | | | | | | | |
| Single | 21 | GCAAUUUUAUCAGCACAAAGA | UCUUUGUGCUGAUAAAAUUGC | [1279-1299] | [1033-1053] | [1336-1354] | [1376-1394] |
| Sp | | | | | | | |
| Single | 21 | GAGUUCUGUUGGCAAUUUUAU | AUAAAAUUGCCAACAGAACUC | [1268-1288] | [1022-1042] | — | — |
| Sp | | | | | | | |
| Single | 21 | CACGAGUUCUGUUGGCAAUUU | AAAUUGCCAACAGAACUCGUG | [1265-1285] | [1019-1039] | — | — |
| Sp | | | | | | | |
| Cross | 21 | CCUUCAACCUCACCCUGUACU | AGUACAGGGUGAGGUUGAAGG | [121-141] | [121-141] | [209-220] | [225-236] |
| Sp | | | | | | | |
| Single | 21 | GAUACUAUCUGGAACUUCCAU | AUGGAAGUUCCAGAUAGUAUC | [999-1019] | [753-773] | — | — |
| Sp | | | | | | | |
| Single | 21 | GAAGAAGGAUACUAUCUGGAA | UUCCAGAUAGUAUCCUUCUUC | [992-1012] | [746-766] | [1047-1067] | — |
| Sp | | | | | | | |
| Single | 21 | UGACAACACAGGCAGGAUUUU | AAAAUCCUGCCUGUGUUGUCA | [962-982] | [716-736] | [1017-1037] | [1058-1075] |
| Sp | | | | | | | |
| Single | 21 | UACGGGCAAUGCUGGGUCUUU | AAAGACCCAGCAUUGCCCGUA | [831-851] | [585-605] | — | [929-946] |
| Sp | | | | | | | |
| Single | 21 | AAUGGAAACUGGAGUGAGAAU | AUUCUCACUCCAGUUUCCAUU | [723-743] | [477-497] | [778-798] | [818-838] |
| Sp | | | | | | | |
| Single | 21 | AUGGGGUGCUCAAUGGAAACU | AGUUUCCAUUGAGCACCCCAU | [712-732] | [466-486] | [772-787] | [812-827] |
| Sp | | | | | | | |
| Single | 21 | CAGCAAUGAUGAUAAUGGGGU | ACCCCAUUAUCAUCAUUGCUG | [698-718] | [452-472] | [753-764] | [793-804] |
| Sp | | | | | | | |
| Single | 21 | UGAUCAACAGCAAUGAUGAUA | UAUCAUCAUUGCUGUUGAUCA | [691-711] | [445-465] | [746-764] | [788-804] |
| Sp | | | | | | | |
| Single | 21 | GUGCCAUGAUCAACAGCAAUG | CAUUGCUGUUGAUCAUGGCAC | [685-705] | [439-459] | — | — |
| Sp | | | | | | | |
| Single | 21 | AGUGGUGUGUGCCAUGAUCAA | UUGAUCAUGGCACACACCACU | [677-697] | [431-451] | [736-749] | [776-789] |
| Sp | | | | | | | |
| Single | 21 | CUAGACAAGAGCCUGCACUUC | GAAGUGCAGGCUCUUGUCUAG | [600-620] | [354-374] | — | — |
| Sp | | | | | | | |
| Single | 21 | GAAGCUGCUAGACAAGAGCCU | AGGCUCUUGUCUAGCAGCUUC | [593-613] | [347-367] | — | — |
| Sp | | | | | | | |
| Single | 21 | UGGACAGUUUGAAGACAAAAU | AUUUUGUCUUCAAACUGUCCA | [557-577] | [311-331] | [816-626] | [656-666] |
| Sp | | | | | | | |
| Single | 21 | AGGAGUAUGUCAUGAAUGAUU | AAUCAUUCAUGACAUACUCCU | [481-501] | [235-255] | — | — |
| Sp | | | | | | | |
| Single | 21 | AGAGGCAGGAGUAUGUCAUGA | UCAUGACAUACUCCUGCCUCU | [475-495] | [229-249] | [530-545] | [570-585] |
| Sp | | | | | | | |
| Single | 21 | CUGUCUACUUGGACAGUGAAC | GUUCACUGUCCAAGUAGACAG | [451-471] | [205-225] | — | — |
| Sp | | | | | | | |
| Single | 21 | UAGGGGAGUUCAUCCUGCUUU | AAAGCAGGAUGAACUCCCCUA | [406-426] | — | [463-479] | — |
| Sp | | | | | | | |
| Single | 21 | CUAGGGGAGUUCAUCCUGCUU | AAGCAGGAUGAACUCCCCUAG | [405-425] | — | [463-479] | — |
| Sp | | | | | | | |
| Single | 21 | GGUACCUCUUGAAAAUCCACA | UGUGGAUUUUCAAGAGGUACC | [349-369] | — | — | — |
| Sp | | | | | | | |
| Single | 21 | GUCGGUACCUCUUGAAAAUCC | GGAUUUUCAAGAGGUACCGAC | [346-366] | — | — | — |
| Sp | | | | | | | |
| Single | 21 | ACAGGAAUGUUUAUGUAGACU | AGUCUACAUAAACAUUCCUGU | [2140-2160] | [1894-1914] | — | — |
| Sp | | | | | | | |
| Single | 21 | GGACAUUAAGGGUUACAGGAA | UUCCUGUAACCCUUAAUGUCC | [2126-2146] | [1880-1900] | — | — |
| Sp | | | | | | | |
| Single | 21 | CAAGUUUAAGGACAUUAAGGG | CCCUUAAUGUCCUUAAACUUG | [2117-2137] | [1871-1891] | — | — |
| Sp | | | | | | | |
| Single | 21 | GAAGCAACAAGUUUAAGGACA | UGUCCUUAAACUUGUUGCUUC | [2110-2130] | [1864-1884] | — | — |
| Sp | | | | | | | |
| Single | 21 | GCAGAUCCAAGCUAAUAUGAG | CUCAUAUUAGCUUGGAUCUGC | [2090-2110] | [1844-1864] | [2143-2156] | [2182-2202] |
| Sp | | | | | | | |
| Single | 21 | CAAGAGUGGACAAAGGCAGAU | AUCUGCCUUUGUCCACUCUUG | [2075-2095] | [1829-1849] | — | [2167-2187] |
| Sp | | | | | | | |
| Single | 21 | CUUCAAGAGUGGACAAAGGCA | UGCCUUUGUCCACUCUUGAAG | [2072-2092] | [1826-1846] | [2125-2141] | [2164-2184] |
| Sp | | | | | | | |
| Single | 21 | UCAAGAAACAGCAGAAAGUCU | AGACUUUCUGCUGUUUCUUGA | [1996-2016] | [1750-1770] | — | — |
| Sp | | | | | | | |
| Single | 21 | GUGGCCUCUUCAAGAAACAGC | GCUGUUUCUUGAAGAGGCCAC | [1987-2007] | [1741-1761] | — | — |
| Sp | | | | | | | |
| Single | 21 | GAAGGAAGUGGCCUCUUCAAG | CUUGAAGAGGCCACUUCCUUC | [1980-2000] | [1734-1754] | [2032-2048] | 12072-2088] |
| Sp | | | | | | | |
| Single | 21 | GCAUCACGAUUAAUGUUCUAG | CUAGAACAUUAAUCGUGAUGC | [1870-1890] | [1624-1644] | — | [1962-1982] |
| Sp | | | | | | | |
| Single | 21 | CAAGCAUCACGAUUAAUGUUC | GAACAUUAAUCGUGAUGCUUG | [1867-1887] | [1621-1641] | — | 11962-1979j |
| Sp | | | | | | | |
| Single | 21 | UCAUCACCUUAUCUUAUCCAA | UUGGAUAAGAUAAGGUGAUGA | [1849-1869] | [1603-1623] | — | — |
| Sp | | | | | | | |
| Single | 21 | CAGACAAGCUGAUCCGCAUCA | UGAUGCGGAUCAGCUUGUCUG | [1777-1797] | [1531-1551] | — | — |
| Sp | | | | | | | |
| Single | 21 | CAACAGACAAGCUGAUCCGCA | UGCGGAUCAGCUUGUCUGUUG | [1774-1794] | [1528-1548] | [1828-1838] | — |
| Sp | | | | | | | |
| Single | 21 | CAGUACCUGUCAACAGACAAG | CUUGUCUGUUGACAGGUACUG | [1764-1784] | [1518-1538] | [1816-1836] | — |
| Sp | | | | | | | |
| Single | 21 | GCCAGUACCUGUCAACAGACA | UGUCUGUUGACAGGUACUGGC | [1762-1782] | [1518-1538] | [1814-1834] | — |
| Sp | | | | | | | |
| Single | 21 | GCAAAAUCUCCUAUUCCCAGU | ACUGGGAAUAGGAGAUUUUGC | [1738-1758] | [1492-1512] | — | — |
| Sp | | | | | | | |
| Single | 21 | CCUGCAAAAUCUCCUAUUCCC | GGGAAUAGGAGAUUUUGCAGG | [1735-1755] | [1489-1509] | — | — |
| Sp | | | | | | | |
| Single | 21 | AAAGACCUACCCCUGCAAAAU | AUUUUGCAGGGGUAGGUCUUU | [1724-1744] | [1478-1498] | [1776-1796] | — |
| Sp | | | | | | | |
| Single | 21 | CAAAGACCUACCCCUGCAAAA | UUUUGCAGGGGUAGGUCUUUG | [1723-1743] | [1477-1497] | [1776-1795] | — |
| Sp | | | | | | | |
| Single | 21 | CACACUCUCUCCUAAAGAAGC | GCUUCUUUAGGAGAGAGUGUG | [1703-1723] | [1457-1477] | — | — |
| Sp | | | | | | | |
| Single | 21 | AUCACACUCUCUCCUAAAGAA | UUCUUUAGGAGAGAGUGUGAU | [1701-1721] | [1447-1475] | — | — |
| Sp | | | | | | | |
| Single | 21 | CAGCGUUCAUCACACUCUCUC | GAGAGAGUGUGAUGAACGCUG | [1693-1713] | [1447-1467] | [1750-1760] | [1785-1800] |
| Sp | | | | | | | |
| Single | 21 | UCAACAUGUCCUCCCAGUUCA | UGAACUGGGAGGACAUGUUGA | [1597-1617] | [1351-1371] | — | — |
| Sp | | | | | | | |
| Single | 21 | CUCAACAUGUCCUCCCAGUUC | GAACUGGGAGGACAUGUUGAG | [1596-1616] | [1350-1370] | — | — |
| Sp | | | | | | | |
| Single | 21 | GCAUGGGCCAGGAUAUAUGCU | AGCAUAUAUCCUGGCCCAUGU | [1561-1581] | [1315-1335] | — | — |
| Sp | | | | | | | |
| Single | 21 | GUGCAAGUCUCCCUGAAAUUC | GAAUUUCAGGGAGACUUGCAC | [1521-1541] | [1275-1295] | — | — |
| Sp | | | | | | | |
| Single | 21 | UGGUGCAAGUCUCCCUGAAAU | AUUUCAGGGAGACUUGCACCA | [1519-1539] | [1273-1293] | — | — |
| Sp | | | | | | | |
| Single | 21 | GAGCAGAGUUGCAACCUUCCA | UGGAAGGUUGCAACUCUGCUC | [1432-1452] | [1186-1206] | — | — |
| Sp | | | | | | | |
| Single | 21 | CCAAAGAGGAGCAGAGUUGCA | UGCAACUCUGCUCCUCUUUGG | [1424-1444] | [1178-1198] | — | — |
| Sp | | | | | | | |
| Single | 21 | UGCAGAAGCUGAAGGCUAGAA | UUCUAGCCUUCAGCUUCUGCA | [1390-1410] | [1144-1164] | — | — |
| Sp | | | | | | | |
| Single | 21 | GCAGGUGUUUCUGAAGGCUCU | AGAGCCUUCAGAAACACCUGC | [1370-1390] | [1124-1144] | [1426-1445] | — |
| Sp | | | | | | | |
| Single | 21 | CAAGUAUGAAGAAGGAUCCCU | AGGGAUCCUUCUUCAUACUUG | [1340-1360] | [1094-1114] | [1395-1415] | [1435-1455] |
| Sp | | | | | | | |
| Single | 21 | ACAGAGAACUACAAGUAUGAA | UUCAUACUUGUAGUUCUCUGU | [1329-1349] | [1063-1103] | [1387-1404] | [1427-1444] |
| Sp | | | | | | | |
| Single | 21 | ACAUCACAGAGAACUACAAGU | ACUUGUAGUUCUCUGUGAUGU | [1324-1344] | [1078-1098] | [1382-1399] | — |
| Sp | | | | | | | |
| Single | 21 | GCACAAAGAGCAUCCAGAGUG | CACUCUGGAUGCUCUUUGUGC | [1291-1311] | [1045-1065] | [1016-1366] | [1386-1406] |
| Sp | | | | | | | |
| Single | 21 | GGACACGAGUUCUGUUGGCAA | UUGCCAACAGAACUCGUGUCC | [1262-1282] | [1016-1036] | — | — |
| Sp | | | | | | | |
| Cross | 21 | UCAACCUCACCCUGUACUUCA | UGAAGUACAGGGUGAGGUUGA | [124-144] | [124-144] | [209-221] | [225-237] |
| Sp | | | | | | | |
| Single | 21 | GGUGAAUGCUGACUGCAUGUC | GACAUGCAGUCAGCAUUCACC | [1202-1222] | [956-976] | [1257-1277] | [1297-1317] |
| Sp | | | | | | | |
| Single | 21 | GAUGGUGAAUGCUGACUGCAU | AUGCAGUCAGCAUUCACCAUC | [1199-1219] | [953-973] | [1255-1274] | [1295-1314] |
| Sp | | | | | | | |
| Single | 21 | UUUGUGUUUUCGAUGGUGAAU | AUUCACCAUCGAAAACACAAA | [1188-1208] | [942-962] | — | — |
| Sp | | | | | | | |
| Single | 21 | ACACGCCCUUUGUGUUUUCGA | UCGAAAACACAAAGGGCGUGU | [1180-1200] | [934-954] | — | — |
| Sp | | | | | | | |
| Single | 21 | CUAUGACACGCCCUUUGUGUU | AACACAAAGGGCGUGUCAUAG | [1175-1195] | [929-949] | — | — |
| Sp | | | | | | | |
| Single | 21 | CCUGAACUAUGACACGCCCUU | AAGGGCGUGUCAUAGUUCAGG | [1169-1189] | [923-943] | [1225-1235] | [1268-1278] |
| Sp | | | | | | | |
| Single | 21 | AAGUGGACCUGAACUAUGACA | UGUCAUAGUUCAGGUCCACUU | [1162-1182] | [916-936] | [1225-1235] | — |
| Sp | | | | | | | |
| Single | 21 | AAGGAGAAGUGGACCUGAACU | AGUUCAGGUCCACUUCUCCUU | [1156-1176] | [910-930] | — | — |
| Sp | | | | | | | |
| Single | 21 | CUAUCUGGAACUUCCAUGUCU | AGACAUGGAAGUUCCAGAUAG | [1003-1023] | [757-777] | — | — |
| Sp | | | | | | | |
| Single | 21 | AUACUAUCUGGAACUUCCAUG | pAUGGAAGUUCCAGAUAGUAU | [1000-1020] | [754-774] | — | — |
| Sp | | | | | | | |
| Single | 21 | UAAGAAGAAGGAUACUAUCUG | CAGAUAGUAUCCUUCUUCUUA | [989-1009] | [743-763] | [1047-1064] | — |
| Sp | | | | | | | |
| Single | 21 | ACACAGGCAGGAUUUUGGGGA | UCCCCAAAAUCCUGCCUGUGU | [967-987] | [721-741] | — | [1062-1075] |
| Sp | | | | | | | |
| Single | 21 | AGAUGAGUAUUAUGACAACAC | GUGUUGUCAUAAUACUCAUCU | [950-970] | [704-724] | [1006-1025] | [1046-1065] |
| Sp | | | | | | | |
| Single | 21 | AUGGAAACCUGAUCAUAGAUG | CAUCUAUGAUCAGGUUUCCAU | [934-954] | [688-708] | [89-1009] | [1029-1049] |
| Sp | | | | | | | |
| Single | 21 | UGAUCACCAACUUCGACUCUG | CAGAGUCGAAGUUGGUGAUCA | [901-921] | [655-675] | [956-976] | [996-1016] |
| Sp | | | | | | | |
| Single | 21 | GAAUUACACAGACGGCGCCAA | UUGGCGCCGUCUGUGUAAUUC | [740-760] | [494-514] | — | — |
| Sp | | | | | | | |
| Single | 21 | AAACUGGAGUGAGAAUUACAC | GUGUAAUUCUCACUCCAGUUU | [728-748] | [482-502] | [784-800] | [824-840] |
| Sp | | | | | | | |
| Single | 21 | GGUGCUCAAUGGAAACUGGAG | CUCCAGUUUCCAUUGAGCACC | [716-736] | [470-490] | [772-791] | [812-831] |
| Sp | | | | | | | |
| Single | 21 | UAAUGGGGUGCUCAAUGGAAA | UUUCCAUUGAGCACCCCAUUA | [710-730] | [464-484] | [772-782] | [812-822] |
| Sp | | | | | | | |
| Single | 21 | UGAUGAUAAUGGGGUGCUCAA | UUGAGCACCCCAUUAUCAUCA | [704-724] | [458-478] | — | — |
| Sp | | | | | | | |
| Single | 21 | CAAUGAUGAUAAUGGGGUGCU | AGCACCCCAUUAUCAUCAUUG | [701-721] | [455-475] | — | — |
| Sp | | | | | | | |
| Single | 21 | UGUGUGCCAUGAUCAACAGCA | UGCUGUUGAUCAUGGCACACA | [682-702] | [436-456] | [737-757] | [777-797] |
| Sp | | | | | | | |
| Single | 21 | AGAGUGGUGUGUGCCAUGAUC | GAUCAUGGCACACACCACUCU | [675-695] | [429-449] | [730-749] | [770-789] |
| Sp | | | | | | | |
| Single | 21 | UCAUAGACAUCUGCCUGAAGC | GCUUCAGGCAGAUGUCUAUGA | [577-597] | [331-351] | [632-645] | — |
| Sp | | | | | | | |
| Single | 21 | CAAAAUCAUAGACAUCUGCCU | AGGCAGAUGUCUAUGAUUUUG | [572-592] | [326-346] | [631-645] | — |
| Sp | | | | | | | |
| Cross | 21 | GAAAUAAUGUGCGGCACCACA | UGUGGUGCCGCACAUUAUUUC | [55-75] | [55-75] | — | — |
| Sp | | | | | | | |
| Single | 21 | AUGGCUUCAUCUACCAAGGCA | UGCCUUGGUAGAUGAAGCCAU | [502-522] | [256-276] | [557-575] | [597-615] |
| Sp | | | | | | | |
| Single | 21 | UGUCAUGAAUGAUUAUGGCUU | AAGCCAUAAUCAUUCAUGACA | [488-508] | [242-262] | [548-563] | [588-603] |
| Sp | | | | | | | |
| Single | 21 | AGGAUGCUGUCUACUUGGACA | UGUCCAAGUAGACAGCAUCCU | [445-465] | [199-219] | — | — |
| Sp | | | | | | | |
| Single | 21 | UCCUGCUUUUCAAUCCCUGGU | ACCAGGGAUUGAAAAGCAGGA | [418-438] | — | [473-493] | [513-533] |
| Sp | | | | | | | |
| Single | 21 | UCAUCCUGCUUUUCAAUCCCU | AGGGAUUGAAAAGCAGGAUGA | [415-435] | — | — | — |
| Sp | | | | | | | |
| Single | 21 | AGUUCAUCCUGCUUUUCAAUC | GAUUGAAAAGCAGGAUGAACU | [412-432] | — | [467-487] | — |
| Sp | | | | | | | |
| Single | 21 | UGAAAAUCCACAUCGACUCCU | AGGAGUCGAUGUGGAUUUUCA | [358-378] | — | — | — |
| Sp | | | | | | | |
| Single | 21 | UCUUGAAAAUCCACAUCGACU | AGUCGAUGUGGAUUUUCAAGA | [355-375] | — | — | — |
| Sp | | | | | | | |
| Single | 21 | UUAAGGACAUUAAGGGUUACA | UGUAACCCUUAAUGUCCUUAA | [2122-2142] | [1876-1896] | — | — |
| Sp | | | | | | | |
| Single | 21 | ACAAGUUUAAGGACAUUAAGG | CCUUAAUGUCCUUAAACUUGU | [2116-2136] | [1870-1890] | — | — |
| Sp | | | | | | | |
| Single | 21 | UGAGAAGCAACAAGUUUAAGG | CCUUAAACUUGUUGCUUCUCA | [2107-2127] | [1861-1881] | — | — |
| Sp | | | | | | | |
| Single | 21 | CAAGCAUCAUUCUGGAGACCG | CGGUCUCCAGAAUGAUGCUUG | [2047-2067] | [1801-1821] | — | — |
| Sp | | | | | | | |
| Single | 21 | AAGCAAGCAUCAUUCUGGAGA | UCUCCAGAAUGAUGCUUGCUU | [2044-2064] | [1798-1818] | — | — |
| Sp | | | | | | | |
| Single | 21 | CAAACCCCAACACCAAGCAAG | CUUGCUUGGUGUUGGGGUUUG | [2030-2050] | [1784-1804] | — | — |
| Sp | | | | | | | |
| Single | 21 | AGUCUUCCUUGGAGUCCUCAA | UUGAGGACUCCAAGGAAGACU | [2012-2032] | [1766-1786] | [2072-0084] | — |
| Sp | | | | | | | |
| Single | 21 | GAAAGUCUUCCUUGGAGUCCU | AGGACUCCAAGGAAGACUUUC | [2009-2029] | [1763-1783] | — | — |
| Sp | | | | | | | |
| Single | 21 | AGCAGAAAGUCUUCCUUGGAG | CUCCAAGGAAGACUUUCUGCU | [2005-2025] | [1759-1779] | — | — |
| Sp | | | | | | | |
| Single | 21 | AGAAACAGCAGAAAGUCUUCC | GGAAGACUUUCUGCUGUUUCU | [1999-2019] | [17-53-1773] | — | — |
| Sp | | | | | | | |
| Single | 21 | UGCUGACUGUGGAAGGAAGUG | CACUUCCUUCCACAGUCAGCA | [1969-1989] | [1723-1743] | [2021-2041] | [2061-2081] |
| Sp | | | | | | | |
| Single | 21 | ACUGUGUGCUGACUGUGGAAG | CUUCCACAGUCAGCACACAGU | [1963-1983] | [1717-1737] | — | [2055-2067] |
| Sp | | | | | | | |
| Single | 21 | UGAGGACUGUGUGCUGACUGU | ACAGUCAGCACACAGUCCUCA | [1958-1978] | [1712-17321 | — | [2050-2067] |
| Sp | | | | | | | |
| Single | 21 | GUGAUAUUUUCAAACCCCCUC | GAGGGGGUUUGAAAAUAUCAC | [1926-1946] | [1680-1700] | [1984-1994] | — |
| Sp | | | | | | | |
| Single | 21 | CGAUUAAUGUUCUAGGAGCAG | CUGCUCCUAGAACAUUAAUCG | [1876-1896] | [1630-1650] | [1929-1946] | [1969-1986] |
| Sp | | | | | | | |
| Single | 21 | CACGAUUAAUGUUCUAGGAGC | GCUCCUAGAACAUUAAUCGUG | [1874-1894] | [1628-1648] | [1929-1946] | [1969-1986] |
| Sp | | | | | | | |
| Single | 21 | AUCCAAGCAUCACGAUUAAUG | CAUUAAUCGUGAUGCUUGGAU | [1864-1884] | [1618-1638] | — | — |
| Sp | | | | | | | |
| Single | 21 | CUUAUCUUAUCCAAGCAUCAC | GUGAUGCUUGGAUAAGAUAAG | [1856-1876 | [1610-1630] | — | — |
| Sp | | | | | | | |
| Single | 21 | AGAUCAUCACCUUAUCUUAUC | GAUAAGAUAAGGUGAUGAUCU | [1846-1866] | [1600-1620] | [1898-1910] | [1938-1950] |
| Sp | | | | | | | |
| Single | 21 | GCAGUCCUGAGAAAAUCCUGG | CCAGGAUUUUCUCAGGACUGC | [1819-1839] | [1573-1593] | [1871-1891] | [1912-1931] |
| Sp | | | | | | | |
| Single | 21 | CUUCGUGGUUGAAACUGGACC | GGUCCAGUUUCAACCACGAAG | [182-202] | [182-198] | — | — |
| Sp | | | | | | | |
| Single | 21 | GAAGAGAAAAGCAGUCCUGAG | CUCAGGACUGCUUUUCUCUUC | [1809-1829] | [1563-1583] | [1861-1881] | — |
| Sp | | | | | | | |
| Single | 21 | UGCCCUGGGUGAAGAGAAAAG | CUUUUCUCUUCACCCAGGGCA | [1799-1819] | [1553-1573] | [1851-1868] | [1891-1908] |
| Sp | | | | | | | |
| Single | 21 | ACAAGCUGAUCCGCAUCAGUG | CACUGAUGCGGAUCAGCUUGU | [1780-1800] | [1534-1554] | — | [1872-1892] |
| Sp | | | | | | | |
| Single | 21 | ACAGCCAGUACCUGUCAACAG | CUGUUGACAGGUACUGGCUGU | [1759-1779] | [1513-1533] | [1811-1831] | — |
| Sp | | | | | | | |
| Single | 21 | AUCUCCUAUUCCCAGUACAGC | GCUGUACUGGGAAUAGGAGAU | [1743-1763] | [1497-1517] | — | — |
| Sp | | | | | | | |
| Single | 21 | ACCUACCCCUGCAAAAUCUCC | GGAGAUUUUGCAGGGGUAGGU | [1728-1748] | [1482-1502] | — | — |
| Sp | | | | | | | |
| Single | 21 | ACACUCUCUCCUAAAGAAGCA | UGCUUCUUUAGGAGAGAGUGU | [1704-1724 | [1458-1478] | — | — |
| Sp | | | | | | | |
| Single | 21 | ACCUCAAAGUGAACCUGAGUG | CACUCAGGUUCACUUUGAGGU | [1621-1641] | [1375-1395] | — | — |
| Sp | | | | | | | |
| Single | 21 | ACAUGUCCUCCCAGUUCAAGG | CCUUGAACUGGGAGGACAUGU | [1600-1620] | [1354-1374] | — | — |
| Sp | | | | | | | |
| Single | 21 | AACAUGUCCUCCCAGUUCAAG | CUUGAACUGGGAGGACAUGUU | [1599-1619] | [1353-1373] | — | — |
| Sp | | | | | | | |
| Single | 21 | CAGGAUAUAUGCUUUGUCCUG | CAGGACAAAGCAUAUAUCCUG | [1569-1589] | [1353-1343] | — | — |
| Sp | | | | | | | |
| Single | 21 | CCCUGAAAUUCAAGCUGCUCG | CGAGCAGCUUGAAUUUCAGGG | [1531-1551] | [1285-1305] | — | — |
| Sp | | | | | | | |
| Single | 21 | AAGUCUCCCUGAAAUUCAAGC | GCUUGAAUUUCAGGGAGACUU | [1525-1545] | [1279-1299] | — | — |
| Sp | | | | | | | |
| Single | 21 | UGAUGUGGUGCAAGUCUCCCU | AGGGAGACUUGCACCACAUCA | [1514-1534] | [1268-1288] | — | — |
| Sp | | | | | | | |
| Single | 21 | AUACACCUUCCCUUCGACCCA | UGGGUCGAAGGGAAGGUGUAU | [1492-1512] | [1246-1266] | [1545-1564] | [1587-1604] |
| Sp | | | | | | | |
| Single | 21 | UUCCAUGGCUCCCAAAGAGGA | UCCUCUUUGGGAGCCAUGGAA | [1413-1433] | [1167-1187] | — | — |
| Sp | | | | | | | |
| Single | 21 | UGAGAAGCAACAAGUUUAAGG | CCUUAAACUUGUUGCUUCUCA | [2107-2127] | [1861-1881] | — | — |
| Sp | | | | | | | |
| Single | 21 | CAAGCAUCAUUCUGGAGACCG | CGGUCUCCAGAAUGAUGCUUG | [2047-2067] | [1801-1821] | — | — |
| Sp | | | | | | | |
| Single | 21 | AAGCAAGCAUCAUUCUGGAGA | UCUCCAGAAUGAUGCUUGCUU | [2044-2064] | [1798-1818] | — | — |
| Sp | | | | | | | |
| Single | 21 | CAAACCCCAACACCAAGCAAG | CUUGCUUGGUGUUGGGGUUUG | [2030-2050] | [1784-1804] | — | — |
| Sp | | | | | | | |
| Single | 21 | AGUCUUCCUUGGAGUCCUCAA | UUGAGGACUCCAAGGAAGACU | [2012-2032] | [1766-1786] | [2072-2084] | — |
| Sp | | | | | | | |
| Single | 21 | GAAAGUCUUCCUUGGAGUCCU | AGGACUCCAAGGAAGACUUUC | [2009-2029] | [1763-1783] | — | — |
| Sp | | | | | | | |
| Single | 21 | AGCAGAAAGUCUUCCUUGGAG | CUCCAAGGAAGACUUUCUGCU | [2005-2025] | [1759-1779] | — | — |
| Sp | | | | | | | |
| Single | 21 | AGAAACAGCAGAAAGUCUUCC | GGAAGACUUUCUGCUGUUUCU | [1999-2019] | [1753-1773] | — | — |
| Sp | | | | | | | |
| Single | 21 | UGCUGACUGUGGAAGGAAGUG | CACUUCCUUCCACAGUCAGCA | [1969-1989] | [1723-1743] | [2021-2041] | [2061-2081] |
| Sp | | | | | | | |
| Single | 21 | ACUGUGUGCUGACUGUGGAAG | CUUCCACAGUCAGCACACAGU | [1963-1983] | [1717-1737] | — | [2055-2057] |
| Sp | | | | | | | |
| Single | 21 | UGAGGACUGUGUGCUGACUGU | ACAGUCAGCACACAGUCCUCA | [1958-1978] | [1717-1732] | — | [2050-2067] |
| Sp | | | | | | | |
| Single | 21 | GUGAUAUUUUCAAACCCCCUC | GAGGGGGUUUGAAAAUAUCAC | [1926-1946] | [1680-1700] | [1984-1994] | — |
| Sp | | | | | | | |
| Single | 21 | CGAUUAAUGUUCUAGGAGCAG | CUGCUCCUAGAACAUUAAUCG | [1876-1896] | [1630-1650] | [1929-1946] | [1969-1986] |
| Sp | | | | | | | |
| Single | 21 | CACGAUUAAUGUUCUAGGAGC | GCUCCUAGAACAUUAAUCGUG | [1874-1894] | [1628-1648] | [1929-1946] | [1969-1986] |
| Sp | | | | | | | |
| Single | 21 | AUCCAAGCAUCACGAUUAAUG | CAUUAAUCGUGAUGCUUGGAU | [1864-1884] | [1618-1638] | — | — |
| Sp | | | | | | | |
| Single | 21 | CUUAUCUUAUCCAAGCAUCAC | GUGAUGCUUGGAUAAGAUAAG | [1856-1876] | [1610-1630] | — | — |
| Sp | | | | | | | |
| Single | 21 | AGAUCAUCACCUUAUCUUAUC | GAUAAGAUAAGGUGAUGAUCU | [1846-1866] | [1600-1620] | [1898-1910] | [1938-1950] |
| Sp | | | | | | | |
| Single | 21 | GCAGUCCUGAGAAAAUCCUGG | CCAGGAUUUUCUCAGGACUGC | [1819-1839] | [1573-1593] | [1871-1891] | [1912-1931] |
| Sp | | | | | | | |
| Single | 21 | CUUCGUGGUUGAAACUGGACC | GGUCCAGUUUCAACCACGAAG | [182-202] | [182-198] | — | — |
| Sp | | | | | | | |
| Single | 21 | GAAGAGAAAAGCAGUCCUGAG | CUCAGGACUGCUUUUCUCUUC | [1809-1829] | [1563-1583] | [1861-1881] | — |
| Sp | | | | | | | |
| Single | 21 | UGCCCUGGGUGAAGAGAAAAG | CUUUUCUCUUCACCCAGGGCA | [1799-1819] | [1553-1573] | [1861-1868] | [1891-1908] |
| Sp | | | | | | | |
| Single | 21 | ACAAGCUGAUCCGCAUCAGUG | CACUGAUGCGGAUCAGCUUGU | [1780-1800] | [1553-1554] | — | [1872-1892] |
| Sp | | | | | | | |
| Single | 21 | ACAGCCAGUACCUGUCAACAG | CUGUUGACAGGUACUGGCUGU | [1759-1779] | [1513-1533] | [1811-1831] | — |
| Sp | | | | | | | |
| Single | 21 | AUCUCCUAUUCCCAGUACAGC | GCUGUACUGGGAAUAGGAGAU | [1743-1763] | [1497-1517] | — | — |
| Sp | | | | | | | |
| Single | 21 | ACCUACCCCUGCAAAAUCUCC | GGAGAUUUUGCAGGGGUAGGU | [1728-1748] | [1482-1502] | — | — |
| Sp | | | | | | | |
| Single | 21 | ACACUCUCUCCUAAAGAAGCA | UGCUUCUUUAGGAGAGAGUGU | [1704-1724] | [1482-1478] | — | — |
| Sp | | | | | | | |
| Single | 21 | ACCUCAAAGUGAACCUGAGUG | CACUCAGGUUCACUUUGAGGU | [1621-1641] | [1375-1395] | — | — |
| Sp | | | | | | | |
| Single | 21 | ACAUGUCCUCCCAGUUCAAGG | CCUUGAACUGGGAGGACAUGU | [1600-1620] | [1354-1374] | — | — |
| Sp | | | | | | | |
| Single | 21 | AACAUGUCCUCCCAGUUCAAG | CUUGAACUGGGAGGACAUGUU | [1599-1619] | [1353-1373] | — | — |
| Sp | | | | | | | |
| Single | 21 | CAGGAUAUAUGCUUUGUCCUG | CAGGACAAAGCAUAUAUCCUG | [1569-1589] | [1323-1343] | — | — |
| Sp | | | | | | | |
| Single | 21 | CCCUGAAAUUCAAGCUGCUCG | CGAGCAGCUUGAAUUUCAGGG | [1531-1551] | [1285-1305] | — | — |
| Sp | | | | | | | |
| Single | 21 | AAGUCUCCCUGAAAUUCAAGC | GCUUGAAUUUCAGGGAGACUU | [1525-1545] | [1279-1299] | — | — |
| Sp | | | | | | | |
| Single | 21 | UGAUGUGGUGCAAGUCUCCCU | AGGGAGACUUGCACCACAUCA | [1514-1534] | [1268-1288] | — | — |
| Sp | | | | | | | |
| Single | 21 | AUACACCUUCCCUUCGACCCA | UGGGUCGAAGGGAAGGUGUAU | [1492-1512] | [1246-1266] | [1545-1564] | [1587-1604] |
| Sp | | | | | | | |
| Single | 21 | UUCCAUGGCUCCCAAAGAGGA | UCCUCUUUGGGAGCCAUGGAA | [1413-1433] | [1167-1187] | — | — |
| Sp | | | | | | | |

TABLE H
TGase 7:19-mers

| | Oligo | | | human | mouse | rat |
|---|---|---|---|---|---|---|
| Source | Length | Sense siRNA | AntiSense iIRNA | 16445034 | 51712629 | 34857657 |
| Single Sp | 19 | GGAACGACGUGGUGUAUGU | ACAUACACCACGUCGUUCC | [652-670] | [2210-2222] | [802-818] |
| Single Sp | 19 | GAACUUUCAUCCUACUUUU | AAAAGUAGGAUGAAAGUUC | [409-4271 | — | — |
| Single Sp | 19 | CCAUGCUGGUCCUAAAAGA | UCUUUUAGGACCAGCAUGG | [1792-1810] | [3349-3362] | [1933-1946] |
| Single Sp | 19 | GGAUGAACCUGGACUUUGG | CCAAAGUCCAGGUUCAUCC | [1660-1678] | — | — |
| Single Sp | 19 | ACAAGAGCCUGUAUCACUU | AAGUGAUACAGGCUCUUGU | [604-622] | — | [752-765] |
| Single Sp | 19 | AUAGACAUCUGCUUUGAGA | UCUCAAAGCAGAUGUCUAU | [579-597] | [2137-21491 | [733-745] |
| Single Sp | 19 | CAAGGGUCAUGAAAGAUUC | GAAUCUUUCAUGACCCUUG | [515-533] | — | — |
| Single Sp | 19 | UUACAAGGGUCAUGAAAGA | UCUUUCAUGACCCUUGUAA | [512-530] | [2065-20751 | [585-596] |
| Single Sp | 19 | CCAACUCUCUCCAAGUUUC | GAAACUUGGAGAGAGUUGG | [298-316] | [1851-1865] | [372-386] |
| Single Sp | 19 | GCAACGAGGUCAAGGAGAU | AUCUCCUUGACCUCGUUGC | [2074-2092] | [3787-3800] | [2215-2228] |
| Single Sp | 19 | UGAGUGCCAUGAUCAACAG | CUGUUGAUCAUGGCACUCA | [682-700] | [2239-2252] | — |
| Single Sp | 19 | AAAGACUGUUCCCAGCGGA | UCCGCUGGGAACAGUCUUU | [636-654] | — | — |
| Single Sp | 19 | UCACCUUUGUGGCUGAGAC | GUCUCAGCCACAAAGGUGA | [178-496] | [1730-1748] | [251-269] |
| Single Sp | 19 | UCAAGGAGAUCAAAGGCUA | UAGCCUUUGAUCUCCUUGA | [2083-2101] | [3791-3809] | [2219-2237] |
| Single Sp | 19 | ACACCUUAAUGGUGGCUCU | AGAGCCACCAUUAAGGUGU | [1900-1918] | — | — |
| Single Sp | 19 | CUAUUGAGGUGUCUGAGAG | CUCUCAGACACCUCAAUAG | [1837-1855] | [3389-3400] | [1973-1984] |
| Single Sp | 19 | AAAGAUUCAUCACCUCCUG | CAGGAGGUGAUGAAUCUUU | [526-544] | — | — |
| Single Sp | 19 | UUAUGGCUUUGUUUACAAG | CUUGUAAACAAAGCCAUAA | [500-518] | — | [574-591] |
| Single Sp | 19 | AAAUACUGCUGCAGGAGUA | UACUCCUGCAGCAGUAUUU | [469-487] | — | — |
| Single Sp | 19 | AUAGAGAUCUCUCAGGGCC | GGCCCUGAGAGAUCUCUAU | [363-381] | [1916-1935] | — |
| Single Sp | 19 | ACAUAUUCGUCACUGUGGC | GCCACAGUGACGAAUAUGU | [2107-2125] | — | — |
| Single Sp | 19 | AAAUUCAACUGGACCUCUA | UAGAGGUCCAGUUGAAUUU | [2011-2029] | — | [2148-2165] |
| Single Sp | 19 | AAAGAUAUCUGUCUGGAGC | GCUCCAGACAGAUAUCUUU | [1806-1824] | — | — |
| Single Sp | 19 | AAAUAGAGAUCUCUCAGGG | CCCUGAGAGAUCUCUAUUU | [361-379] | [1916-1931] | — |
| Single Sp | 19 | AUAUCUGUCUGGAGCCUCC | GGAGGCUCCAGACAGAUAU | [1810-1828] | [3369-3380] | [1953-1964] |
| Single Sp | 19 | CAACAGCAACGAUGACAAU | AUUGUCAUCGUUGCUGUUG | [695-713] | — | — |
| Single Sp | 19 | GCUUUGAGAUCCUGAACAA | UUGUUCAGGAUCUCAAAGC | [589-607] | [2141-2159] | [737-755] |
| Single Sp | 19 | GGAACUACGGGCAGUUUGA | UCAAACUGCCCGUAGUUCC | [550-568] | [2102-2120] | [823-636] |
| Single Sp | 19 | CGAGAUUAUGGCUUUGUUU | AAACAAAGCCAUAAUCUCG | [495-513] | — | [574-586] |
| Single Sp | 19 | GCGAGAUUAUGGCUUUGUU | AACAAAGCCAUAAUCUCGC | [494-512] | — | [574-585] |
| Single Sp | 19 | CCAUUACACUCUGAAAAUA | UAUUUUCAGAGUGUAAUGG | [347-365] | — | — |
| Single Sp | 19 | GCAGUUAUUGGCCAUUACA | UGUAAUGGCCAAUAACUGC | [336-354] | — | — |
| Single Sp | 19 | CCAAGUUUCCCUUUUCACA | UGUGAAAAGGGAAACUUGG | [308-326] | — | — |
| Single Sp | 19 | CUCCAAGUUUCCCUUUUCA | UGAAAAGGGAAACUUGGAG | [306-324] | — | — |
| Single Sp | 19 | CUCCAACUCUCUCCAAGUU | AACUUGGAGAGAGUUGGAG | [296-314] | [1851-1865] | [372-386] |
| Single Sp | 19 | CUUCUGAUUUCACCAUUGA | UCAAUGGUGAAAUCAGAAG | [277-295] | — | [612-698] |
| Single Sp | 19 | GACACACCCUCCAAAUUCA | UGAAUUUGGAGGGUGUGUC | [1999-2017] | — | — |
| Single Sp | 19 | CUCCCCACUUGUCUAUUGA | UCAAUAGACAAGUGGGGAG | [1825-1843] | [3377-33951 | [1961-1979] |
| Single Sp | 19 | GGUCCAUGCUGGUCCUAAA | UUUAGGACCAGCAUGGACC | [1789-1807] | [3349-3359] | [1933-1943] |
| Single Sp | 19 | CAAGCUAACGGACGAAAAG | CUUUUCGUCCGUUAGCUUG | [1727-1744] | — | — |
| Single Sp | 19 | ACAAGCUAACGGACGAAAA | UUUUCGUCCGUUAGCUUGU | [1726-1744] | — | — |
| Single Sp | 19 | CUACAGCAAUUACAGAAAC | GUUUCUGUAAUUGCUGUAG | [1709-1727] | — | [1845-1859] |
| Single Sp | 19 | CAGCAACGAUGACAAUGGC | GCCAUUGUCAUCGUUGCUG | [1698-7161] | — | — |
| Single Sp | 19 | UCAACAGCAACGAUGACAA | UUGUCAUCGUUGCUGUUGA | [694-712] | — | — |
| Single Sp | 19 | GUGCCAUGAUCAACAGCAA | UUGCUGUUGAUCAUGGCAC | [685-703] | [2239-2255] | [827-839] |
| Single Sp | 19 | GAGCCUGUAUCACUUAAAG | CUUUAAGUGAUACAGGCUC | [608-626] | — | |
| Single Sp | 19 | UGAACAAGAGCCUGUAUCA | UGAUACAGGCUCUUGUUCA | [601-619] | [2153-2165] | [749-765] |
| Single Sp | 19 | UCAUAGACAUCUGCUUUGA | UCAAAGCAGAUGUCUAUGA | [577-595] | [2137-2147] | [733-743] |
| Single Sp | 19 | GGACAUCAUAGACAUCUGC | GCAGAUGUCUAUGAUGUCC | [572-590] | — | — |
| Single Sp | 19 | AAGAGGACAUCAUAGACAU | AUGUCUAUGAUGUCCUCUU | [568-586] | [2120-2130] | [716-726] |
| Single Sp | 19 | GGAACAACAAGGAGCACCA | UGGUGCUCCUUGUUGUUCC | [58-76] | [1610-1628] | [131-149] |
| Single Sp | 19 | CCAGGAACAACAAGGAGCA | UGCUCCUUGUUGUUCCUGG | [55-73] | [1607-1622] | [128-143] |
| Single Sp | 19 | GUAUAUCAUGCGAGAUUAU | AUAAUCUCGCAUGAUAUAC | [485-503] | — | — |
| Single Sp | 19 | GCAGGAGUAUAUCAUGCGA | UCGCAUGAUAUACUCCUGC | [479-497] | [2034-2046] | [555-567] |
| Single Sp | 19 | GUGAAAUACUGCUGCAGGA | UCCUGCAGCAGUAUUUCAC | [466-484] | — | |
| Single Sp | 19 | CAAGUGAAAUACUGCUGCA | UGCAGCAGUAUUUCACUUG | [463-481] | — | — |
| Single Sp | 19 | CCUGCCAAGUGAAAUACUG | CAGUAUUUCACUUGGCAGG | [458-476] | [2010-2023] | — |
| Single Sp | 19 | ACGUCUACCUGCCAAGUGA | UCACUUGGCAGGUAGACGU | [451-469] | [2005-2021] | [526-536] |
| Single Sp | 19 | AGAGCUCCAGGAACAACAA | UUGUUGUUCCUGGAGCUCU | [49-67] | [1607-1619] | [128-140] |
| Single Sp | 19 | GUCACAGUGUGACUUACCC | GGGUAAGUCACACUGUGAC | [385-403] | [1943-1955] | — |
| Single Sp | 19 | CCAAUGCAGUUAUUGGCCA | UGGCCAAUAACUGCAUUGG | [331-349] | — | — |
| Single Sp | 19 | ACCAGCCAAUGCAGUUAUU | AAUAACUGCAUUGGCUGGU | [326-344] | — | — |
| Single Sp | 19 | CAACUCUCUCCAAGUUUCC | GGAAACUUGGAGAGAGUUG | [299-317] | [1851-1865] | [372-386] |
| Single Sp | 19 | GACUCCAACUCUCUCCAAG | CUUGGAGAGAGUUGGAGUC | [294-312] | [1851-1864] | [372-385] |
| Single Sp | 19 | CCAUUGACUCCAACUCUCU | AGAGAGUUGGAGUCAAUGG | [289-307] | — | |
| Single Sp | 19 | GAGCGCUUCUGAUUUCACC | GGUGAAAUCAGAAGCGCUC | [272-290] | — | [682-693] |
| Single Sp | 19 | CGAGCCACAUUCUUCCUCA | OGAGGAAGAAUGUGGCUCG | [228-246] | — | — |
| Single Sp | 19 | ACGACCACAUCACCUUUGU | ACAAAGGUGAUGUGGUCGU | [169-187] | — | — |
| Single Sp | 19 | GAAUGAAGCUCUGUUAGAA | UUCUAACAGAGCUUCAUUC | [2251-2269] | — | — |
| Single Sp | 19 | GCCUGGGAAUGAAUGAAGC | GCUUCAUUCAUUCCCAGGC | [2241-2259] | — | — |
| Single Sp | 19 | CUACUCCUGGCUAUGUCGU | ACGACAUAGCCAGGAGUAG | [2191-2209] | — | — |
| Single Sp | 19 | CCUUUCUACUCCUGGCUAU | AUAGCCAGGAGUAGAAAGG | [2186-2204] | — | — |
| Single Sp | 19 | GGACAUAUUCGUCACUGUG | CACAGUGACGAAUAUGUCC | [2105-2123] | — | — |
| Single Sp | 19 | GGAGAUCAAAGGCUACAAG | CUUGUAGCCUUUGAUCUCC | [2087-2105] | [3795-3813] | [2223-2241] |
| Single Sp | 19 | ACACACCCUCCAAAUUCAA | UUGAAUUUGGAGGGUGUGU | [2000-2018] | — | — |
| Single Sp | 19 | UCAAUGGGCAGAUAGCAAA | UUUGCUAUCUGCCCAUUGA | [1957-1975] | [3509-3521] | [2093-2105] |
| Single Sp | 19 | CCUUAAUGGUGGCUCUGAG | CUCAGAGCCACCAUUAAGG | [1903-1921] | — | — |
| Single Sp | 19 | UCACCAACACCUUAAUGGU | ACCAUUAAGGUGUUGGUGA | [1894-1912] | [3446-3456] | [2030-2048] |
| Single Sp | 19 | CCCCACUUGUCUAUUGAGG | CCUCAAUAGACAAGUGGGG | [1827-1845] | [3379-3397] | [1967-1981] |
| Single Sp | 19 | UGGAUCAGGUGGCAACCUU | AAGGUUGCCACCUGAUCCA | [7-25] | — | — |
| Single Sp | 19 | GAAACAAGCUAACGGACGA | UCGUCCGUUAGCUUGUUUC | [1723-1741] | — | — |
| Single Sp | 19 | ACAGAAACAAGCUAACGGA | UCCGUUAGCUUGUUUCUGU | [1720-1738] | — | — |
| Single Sp | 19 | UGAACCUGGACUUUGGGAA | UUCCCAAAGUCCAGGUUCA | [1663-1681] | — | — |
| Single Sp | 19 | ACACAGUGCGGAUGAACCU | AGGUUCAUCCGCACUGUGU | [1651-1669] | — | — |
| Single Sp | 19 | UGAUCAACAGCAACGAUGA | UCAUCGUUGCUGUUGAUCA | [691-709] | [2243-2255] | [827-839] |
| Single Sp | 19 | ACGACGUGGUGUAUGUGUG | CACACAUACACCACGUCGU | [655-673] | [2210-2225] | [803-818] |
| Single Sp | 19 | AAAGAACCCGGCCAAAGAC | GUCUUUGGCCGGGUUCUUU | [623-641] | — | — |
| Single Sp | 19 | UAAAGAACCCGGCCAAAGA | UCUUUGGCCGGGUUCUUUA | [622-640] | — | — |
| Single Sp | 19 | AGAUCCUGAACAAGAGCCU | AGGCUCUUGUUCAGGAUCU | [595-613] | [2147-2165] | [743-761] |
| Single Sp | 19 | UUGAGAUCCUGAACAAGAG | CUCUUGUUCAGGAUCUCAA | [592-610] | [2144-2162] | — |
| Single Sp | 19 | ACAUCUGCUUUGAGAUCCU | AGGAUCUCAAAGCAGAUGU | [583-601] | [2137-2153] | [733-747] |
| Single Sp | 19 | ACAUCAUAGACAUCUGCUU | AAGCAGAUGUCUAUGAUGU | [574-592] | — | — |
| Single Sp | 19 | AGGAGUAUAUCAUGCGAGA | UCUCGCAUGAUAUACUCCU | [481-499] | [2034-2046] | [555-567] |
| Single Sp | 19 | UCAUCCUACUUUUUAACCC | GGGUUAAAAAGUAGGAUGA | [415-433] | [1975-1985] | [496-506] |
| Single Sp | 19 | UGGGAACUUUCAUCCUACU | AGUAGGAUGAAAGUUCCCA | [406-424] | — | — |
| Single Sp | 19 | UUACCCGCUGGGAACUUUC | GAAAGUUCCCAGCGGGUAA | [398-416] | — | — |
| Single Sp | 19 | AGAGAUCUCUCAGGGCCAA | UUGGCCCUGAGAGAUCUCU | [365-383] | [1917-1934] | — |
| Single Sp | 19 | UGAAAAUAGAGAUCUCUCA | UGAGAGAUCUCUAUUUUCA | [358-376] | [1916-1928] | — |
| Single Sp | 19 | UGCAGUUAUUGGCCAUUAC | GUAAUGGCCAAUAACUGCA | [335-353] | — | — |
| Single Sp | 19 | CAAUGCAGUUAUUGGCCAU | AUGGCCAAUAACUGCAUUG | [332-350] | — | — |
| Single Sp | 19 | UCACACCAGCCAAUGCAGU | ACUGCAUUGGCUGGUGUGA | [322-340] | — | — |
| Single Sp | 19 | ACCAUUGACUCCAACUCUC | bAGAGUUGGAGUCAAUGGU | [288-306] | — | — |
| Single Sp | 19 | UCACCAUUGACUCCAACUC | GAGUUGGAGUCAAUGGUGA | [286-304] | — | — |
| Single Sp | 19 | UUUCACCAUUGACUCCAAC | GUUGGAGUCAAUGGUGAAA | [284-302] | — | — |
| Single Sp | 19 | UUGAGUCUGUCGACCUGCA | UGCAGGUCGACAGACUCAA | [31-49] | — | — |
| Single Sp | 19 | GAGCCACAUUCUUCCUCAC | GUGAGGAAGAAUGUGGCUC | [229-247] | — | — |
| Single Sp | 19 | ACAUCACCUUUGUGGCUGA | UCAGCCACAAAGGUGAUGU | [175-193] | [1730-1745] | [251-266] |
| Single Sp | 19 | AGAACGACCACAUCACCUU | AAGGUGAUGUGGUCGUUCU | [166-184] | — | — |
| Single Sp | 19 | ACACCGUGUGCUUUGGGAA | UUCCCAAAGCACACGGUGU | [2270-2288] | — | — |
| Single Sp | 19 | AACACCGUGUGCUUUGGGA | OCCCAAAGCACACGGUGUU | [2269-2287] | — | — |
| Single Sp | 19 | GAAACACCGUGUGCUUUGG | CCAAAGCACACGGUGUUUC | [2267-2285] | — | — |
| Single Sp | 19 | AUAGAAACACCGUGUGCUU | AAGCACACGGUGUUUCUAA | [2264-2282] | — | — |
| Single Sp | 19 | AUGAAGCUCUGUUAGAAAC | GUUUCUAACAGAGCUUCAU | [2253-2271] | — | — |
| Single Sp | 19 | GGGAAUGAAUGAAGCUCUG | CAGAGCUUCAUUCAUUCCC | [2245-2263] | — | — |
| Single Sp | 19 | UCUGUCCUCUCUCUAGCCU | AGGCUAGAGAGAGGACAGA | [2222-2240] | — | — |
| Single Sp | 19 | UCCUGGCUAUGUCGUCUUG | CAAGACGACAUAGCCAGGA | [2195-2213] | — | — |
| Single Sp | 19 | UUCUACUCCUGGCUAUGUC | GACAUAGCCAGGAGUAGAA | [2189-2207] | — | — |
| Single Sp | 19 | AGGACAUAUUCGUCACUGU | ACAGUGACGAAUAUGUCCU | [2104-2122] | — | — |
| Single Sp | 19 | UCAAAGGCUACAAGGACAU | AUGUCCUUGUAGCCUUUGA | [2092-2110] | [3800-3818] | [2228-2246] |
| Single Sp | 19 | AGAUCAAAGGCUACAAGGA | UCCUUGUAGCCUUUGAUCU | [2089-2107] | [3797-3815] | [2225-2243] |
| Single Sp | 19 | UCAGCAGCAACGAGGUCAA | UUGACCUCGUUGCUGCUGA | [2068-2086] | — | — |
| Single Sp | 19 | UCAUCAGCAGCAACGAGGU | ACCUCGUUGCUGCUGAUGA | [2065-2083] | [3773-3784] | [2201-2212] |
| Single Sp | 19 | CCUCCAAAUUCAACUGGAC | GUCCAGUUGAAUUUGGAGG | [2006-2024] | — | [2148-2159] |
| Single Sp | 19 | AAAGGACCUUGGGACUCUG | CAGAGUCCCAAGGUCCUUU | [1973-1991] | [3687-3699] | [2113-2127] |
| Single Sp | 19 | UCAUCAAUGGGCAGAUAGC | GCUAUCUGCCCAUUGAUGA | [1954-1972] | [3506-3521] | [2090-2105] |
| Single Sp | 19 | CCAACACCUUAAUGGUGGC | GCCACCAUUAAGGUGUUGG | [1897-1915] | — | — |
| Single Sp | 19 | UCACCCUCACCAACACCUU | AAGGUGUUGGUGAGGGUGA | [1888-1906] | [3440-3456] | [2024-2040] |
| Single Sp | 19 | AUGUCACCCUCACCAACAC | GUGUUGGUGAGGGUGACAU | [1885-1903] | [1440-3455] | [2024-2039] |
| Single Sp | 19 | CCACUUGUCUAUUGAGGUG | CACCUCAAUAGACAAGUGG | [1829-1847] | [13381-3399] | [1967-1983] |
| Single Sp | 19 | AGAUAUCUGUCUGGAGCCU | AGGCUCCAGACAGAUAUCU | [1808-1826] | — | — |
| Single Sp | 19 | AAAGCUCAUCCGCGUGUCU | AGACACGCGGAUGAGCUUU | [1742-1760] | — | |
| Single Sp | 19 | ACGAAAAGCUCAUCCGCGU | ACGCGGAUGAGCUUUUCGU | [1738-1756] | — | — |
| Single Sp | 19 | ACAACAAGGAGCACCACAC | GUGUGGUGCUCCUUGUUGU | [61-79] | — | — |
| Single Sp | 19 | AGAUUCAUCACCUCCUGGC | UCCAGGAGGUGAUGAAUCU | [528-546] | — | — |
| Single Sp | 19 | AUGAAAGAUUCAUCACCUC | GAGGUGAUGAAUCUUUCAU | [523-541] | — | — |
| Single Sp | 19 | UUUCCCUUUUCACACCAGC | GCUGGUGUGAAAAGGGAAA | [313-331] | — | — |
| Single Sp | 19 | UGGCUCCUUUCUACUCCUG | CAGGAGUAGAAAGGAGCCA | [2181-2199] | — | — |
| Single Sp | 19 | ACAAGGACAUAUUCGUCAC | GUGACGAAUAUGUCCUUGU | [2101-2119] | — | — |
| Single Sp | 19 | UCCAGGUUCUCAUCAGCAG | CUGCUGAUGAGAACCUGGA | [2056-2074] | [3772-3782] | [2200-2210] |
| Single Sp | 19 | AGAUAGCAAAGGACCUUGG | CCAAGGUCCUUUGCUAUCU | [1966-1984] | — | — |
| Single Sp | 19 | UUAAUGGUGGCUCUGAGCA | UGCUCAGAGCCACCAUUAA | [1905-1923] | — | — |
| Single Sp | 19 | UAAAAGAUAUCUGUCUGGA | UCCAGACAGAUAUCUUUUA | [1180-1822] | — | — |
| Single Sp | 19 | UUUAACCCUUGGAGUCCAG | CUGGACUCCAAGGGUUAAA | [426-444] | — | — |
| Single Sp | 19 | AAAAGCUCAUCCGCGUGUC | GACACGCGGAUGAGCUUUU | [1741-1759] | — | — |
| Single Sp | 19 | CUGCCCUACAGCAAUUACA | UGUAAUUGCUGUAGGGCAG | [1704-1722] | [3256-3274] | [1840-1858] |
| Single Sp | 19 | CUCCUGCCCUACAGCAAUU | AAUUGCUGUAGGGCAGGAG | [1701-1719] | [3256-3268] | [1840-1855] |
| Single Sp | 19 | GUGGUGAGUGCCAUGAUCA | UGAUCAUGGCACUCACCAC | [678-696] | — | — |
| Single Sp | 19 | GCCUGUAUCACUUAAAGAA | UUCUUUAAGUGAUACAGGC | [610-628] | — | — |
| Single Sp | 19 | CAAGAGCCUGUAUCACUUA | UAAGUGAUACAGGCUCUUG | [605-623] | — | [753-765] |
| Single Sp | 19 | GAAGAGGACAUCAUAGACA | UGUCUAUGAUGUCCUCUUC | [567-585] | [2119-2130] | [715-726] |
| Single Sp | 19 | GAACUACGGGCAGUUUGAA | UUCAAACUGCCCGUAGUUC | [551-569] | [2103-2121] | [824-836] |
| Single Sp | 19 | GGUCAUGAAAGAUUCAUCA | UGAUGAAUCUUUCAUGACC | [519-537] | — | — |
| Single Sp | 19 | GAGAUUAUGGCUUUGUUUA | UAAACAAAGCCAUAAUCUC | [496-514] | — | [574-587] |
| Single Sp | 19 | CUACCUGCCAAGUGAAAUA | UAUUUCACUUGGCAGGUAG | [455-473] | [2007-2023] | — |
| Single Sp | 19 | GUCUACCUGCCAAGUGAAA | UUUCACUUGGCAGGUAGAC | [453-471] | [2005-2023] | [526-536] |
| Single Sp | 19 | CGUCUACCUGCCAAGUGAA | UUCACUUGGCAGGUAGACG | [452-470] | [2005-2022] | [526-536] |
| Single Sp | 19 | CAGAGCUCCAGGAACAACA | UGUUGUUCCUGGAGCUCUG | [48-66] | [1607-1618] | [128-139] |
| Single Sp | 19 | GGAACUUUCAUCCUACUUU | AAAGUAGGAUGAAAGUUCC | [408-426] | — | — |
| Single Sp | 19 | GCUGGGAACUUUCAUCCUA | OAGGAUGAAAGUUCCCAGC | [404-422] | — | — |
| Single Sp | 19 | CAAGGUCACAGUGUGACUU | AAGUCACACUGUGACCUUG | [381-399] | — | — |
| Single Sp | 19 | GGCCAUUACACUCUGAAAA | UUUUCAGAGUGUAAUGGCC | [345-363] | — | — |
| Single Sp | 19 | CUCUCCAAGUUUCCCUUUU | AAAAGGGAAACUUGGAGAG | [304-322] | — | — |
| Single Sp | 19 | CGCUUCUGAUUUCACCAUU | AAUGGUGAAAUCAGAAGCG | [275-293] | — | [682-693] |
| Single Sp | 19 | GUCUGGAGCGCUUCUGAUU | AAUCAGAAGCGCUCCAGAC | [267-285] | — | — |
| Single Sp | 19 | GAACGACCACAUCACCUUU | AAAGGUGAUGUGGUCGUUC | [167-185] | — | — |
| Single Sp | 19 | GAAGAGACAAUAAAGAUGU | ACAUCUUUAUUGUCUCUUC | [2286-2304] | — | — |
| Single Sp | 19 | CGUGUGCUUUGGGAAGAGA | UCUCUUCCCAAAGCACACG | [2274-2292] | — | — |
| Single Sp | 19 | CACCUCUGUCCUCUCUCUA | UAGAGAGAGGACAGAGGUG | [2218-2236] | — | — |
| Single Sp | 19 | CACCUGGCUCCUUUCUACU | AGUAGAAAGGAGCCAGGUG | [2177-2195] | — | — |
| Single Sp | 19 | GCUACAAGGACAUAUUCGU | ACGAAUAUGUCCUUGUAGC | [2098-2116] | [3806-3818] | [2234-2246] |
| Single Sp | 19 | CGAGGUCAAGGAGAUCAAA | UUUGAUCUCCUUGACCUCG | [2078-2096] | [3787-3804] | [2215-2232] |
| Single Sp | 19 | CGGACACACCCUCCAAAUU | AAUUUGGAGGGUGUGUCCG | [1997-2015] | — | — |
| Single Sp | 19 | CCUCAUCAAUGGGCAGAUA | UAUCUGCCCAUUGAUGAGG | [1952-1970] | [1504-3521] | [2088-2105] |
| Single Sp | 19 | CCCUCACCAACACCUUAAU | AUUAAGGUGUUGGUGAGGG | [1891-1909] | [1443-3456] | [2027-2045] |
| Single Sp | 19 | GGUCCUAAAAGAUAUCUGU | ACAGAUAUCUUUUAGGACC | [1799-1817] | [3351-3363] | [1935-1946] |
| Single Sp | 19 | CAUGCUGGUCCUAAAAGAU | AUCUUUUAGGACCAGCAUG | [1793-1811] | [3349-3363] | [1933-1946] |
| Single Sp | 19 | GUCCAUGCUGGUCCUAAAA | IJUUUAGGACCAGCAUGGAC | [1790-1808] | [3349-3360] | [1933-1944] |
| Single Sp | 19 | GCUAACGGACGAAAAGCUC | GAGCUUUUCGUCCGUUAGC | [1730-1748] | — | — |
| Single Sp | 19 | AGCUAACGGACGAAAAGCU | AGCUUUUCGUCCGUUAGCU | [1729-1747] | — | — |
| Single Sp | 19 | CAAUUACAGAAACAAGCUA | UAGCUUGUUUCUGUAAUUG | [1715-1733] | — | — |
| Single Sp | 19 | GCAAUUACAGAAACAAGCU | AGCUUGUUUCUGUAAUUGC | [1714-1732] | — | — |
| Single Sp | 19 | ACAGCAAUUACAGAAACAA | UUGUUUCUGUAAUUGCUGU | [1711-1729] | — | [1847-1859] |
| Single Sp | 19 | UACAGCAAUUACAGAAACA | UGUUUCUGUAAUUGCUGUA | [1710-1728] | — | [1846-1859] |
| Single Sp | 19 | CUGGACUUUGGGAAGGAGA | UCUCCUUCCCAAAGUCCAG | [1668-1686] | — | — |
| Single Sp | 19 | GAUGAACCUGGACUUUGGG | CCCAAAGUCCAGGUUCAUC | [1661-1679] | — | — |
| Single Sp | 19 | CCAUGAUCAACAGCAACGA | 1UCGUUGCUGUUGAUCAUGG | [688-706] | [2240-2255] | [827-839] |
| Single Sp | 19 | UGGUGAGUGCCAUGAUCAA | UUGAUCAUGGCACUCACCA | [679-697] | [2239-2249] | — |
| Single Sp | 19 | GAACGACGUGGUGUAUGUG | CACAUACACCACGUCGUUC | [653-671] | [2210-2223] | [802-818] |
| Single Sp | 19 | AAGAACCCGGCCAAAGACU | AGUCUUUGGCCGGGUUCUU | [624-642] | — | — |
| Single Sp | 19 | CUGUAUCACUUAAAGAACC | GGUUCUUUAAGUGAUACAG | [612-630] | — | — |
| Single SP | 19 | AGAGCCUGUAUCACUUAAA | UUUAAGUGAUACAGGCUCU | [607-625] | — | [55-76] |
| Single Sp | 19 | AAGAGCCUGUAUCACUUAA | UUAAGUGAUACAGGCUCUU | [606-624] | — | [754-765] |
| Single Sp | 19 | GAACAAGAGCCUGUAUCAC | GUGAUACAGGCUCUUGUUC | [602-620] | [2154-2165] | [750-765] |
| Single Sp | 19 | CUGAACAAGAGCCUGUAUC | GAUACAGGCUCUUGUUCAG | [600-618] | [2152-2165] | [749-765] |
| Single Sp | 19 | CAUCUGCUUUGAGAUCCUG | CAGGAUCUCAAAGCAGAUG | [584-602] | [2137-2154] | [733-747] |
| Single Sp | 19 | UAGACAUCUGCUUUGAGAU | AUCUCAAAGCAGAUGUCUA | [580-598] | [2137-2150] | [733-746] |
| Single Sp | 19 | UUGAAGAGGACAUCAUAGA | UCUAUGAUGUCCUCUUCAA | [565-583] | [2117-2130] | [713-726] |
| Single Sp | 19 | GUUUGAAGAGGACAUCAUA | UAUGAUGUCCUCUUCAAAC | [563-581] | [2115-2130] | [711-726] |
| Single Sp | 19 | AGUUUGAAGAGGACAUCAU | AUGAUGUCCUCUUCAAACU | [562-580] | [2114-2130] | [710-726] |
| Single Sp | 19 | GCAGUUUGAAGAGGACAUC | GAUGUCCUCUUCAAACUGC | [560-578] | [2112-2130] | [710-726] |
| Single Sp | 19 | CUACGGGCAGUUUGAAGAG | CUCUUCAAACUGCCCGUAG | [554-572] | [2110-2124] | [710-720] |
| Single Sp | 19 | ACUACGGGCAGUUUGAAGA | UCUUCAAACUGCCCGUAGU | [553-571] | [2105-2123] | [626-636] |
| Single Sp | 19 | CAUGAAAGAUUCAUCACCU | AGGUGAUGAAUCUUUCAUG | [522-540] | — | — |
| Single Sp | 19 | ACAAGGGUCAUGAAAGAUU | AAUCUUUCAUGACCCUUGU | [514-532] | — | — |
| Single Sp | 19 | GUUUACAAGGGUCAUGAAA | UUUCAUGACCCUUGUAAAC | [510-528] | [2065-2075] | [583-596] |
| Single Sp | 19 | UGUUUACAAGGGUCAUGAA | UUCAUGACCCUUGUAAACA | [509-527] | [2065-2075] | [582-596] |
| Single Sp | 19 | GCUUUGUUUACAAGGGUCA | UGACCCUUGUAAACAAAGC | [505-523] | [2065-2075] | [578-596] |
| Single Sp | 19 | CAUGCGAGAUUAUGGCUUU | AAAGCCAUAAUCUCGCAUG | [491-509] | — | — |
| Single Sp | 19 | AGUAUAUCAUGCGAGAUUA | UAAUCUCGCAUGAUAUACU | [484-502] | [2036-2046] | [557-567] |
| Single Sp | 19 | CUGCAGGAGUAUAUCAUGC | GCAUGAUAUACUCCUGCAG | [477-495] | [2029-2046] | [550-567] |
| Single Sp | 19 | UGCUGCAGGAGUAUAUCAU | AUGAUAUACUCCUGCAGCA | [475-493] | [2029-2045] | [548-566] |
| Single Sp | 19 | UACUGCUGCAGGAGUAUAU | AUAUACUCCUGCAGCAGUA | [472-490] | — | — |
| Single Sp | 19 | GCUCCAGGAACAACAAGGA | UCCUUGUUGUUCCUGGAGC | [52-70] | [1607-1622] | [128-143] |
| Single Sp | 19 | CUACUUUUUAACCCUUGGA | UCCAAGGGUUAAAAAGUAG | [420-438] | [1975-1990] | [496-511] |
| Single Sp | 19 | CAUCCUACUUUUUAACCCU | AGGGUUAAAAAGUAGGAUG | [416-434] | [1975-1985] | [496-506] |
| Single Sp | 19 | CUGGGAACUUUCAUCCUAC | GUAGGAUGAAAGUUCCCAG | [405-423] | — | — |
| Single Sp | 19 | UACCCGCUGGGAACUUUCA | UGAAAGUUCCCAGCGGGUA | [399-417] | — | — |
| Single Sp | 19 | CUUACCCGCUGGGAACUUU | AAAGUUCCCAGCGGGUAAG | [397-415] | — | — |
| Single Sp | 19 | CCAAGGUCACAGUGUGACU | AGUCACACUGUGACCUUGG | [380-398] | — | — |
| Single Sp | 19 | CUCUGAAAAUAGAGAUCUC | GAGAUCUCUAUUUUCAGAG | [355-373] | — | — |
| Single Sp | 19 | ACACUCUGAAAAUAGAGAU | AUCUCUAUUUUCAGAGUGU | [352-370] | — | — |
| Single Sp | 19 | UUGGCCAUUACACUCUGAA | UUCAGAGUGUAAUGGCCAA | [343-361] | — | — |
| Single Sp | 19 | AGUUAUUGGCCAUUACACU | AGUGUAAUGGCCAAUAACU | [338-356] | — | — |
| Single Sp | 19 | CAGUUAUUGGCCAUUACAC | GUGUAAUGGCCAAUAACUG | [337-355] | — | — |
| Single Sp | 19 | CCAGCCAAUGCAGUUAUUG | CAAUAACUGCAUUGGCUGG | [327-345] | — | — |
| Single Sp | 19 | UUUCACACCAGCCAAUGCA | UGCAUUGGCUGGUGUGAAA | [320-338] | — | — |
| Single Sp | 19 | CCUUUUCACACCAGCCAAU | AUUGGCUGGUGUGAAAAGG | [317-335] | — | — |
| Single Sp | 19 | CAAGUUUCCCUUUUCACAC | GUGUGAAAAGGGAAACUUG | [309-327] | — | — |
| Single Sp | 19 | CUCUCUCCAAGUUUCCCUU | AAGGGAAACUUGGAGAGAG | [302-320] | [1854-1865] | [375-386] |
| Single Sp | 19 | CUGAUUUCACCAUUGACUC | GAGUCAAUGGUGAAAUCAG | [280-298] | — | [683-698] |
| Single Sp | 19 | UCUGAUUUCACCAUUGACU | AGUCAAUGGUGAAAUCAGA | [279-2971 | — | [682-698] |
| Single Sp | 19 | GCUUCUGAUUUCACCAUUG | CAAUGGUGAAAUCAGAAGC | [276-294] | — | [682-693] |
| Single Sp | 19 | GGAGCGCUUCUGAUUUCAC | GUGAAAUCAGAAGCGCUCC | [271-289] | — | [682-692] |
| Single Sp | 19 | UGGAGCGCUUCUGAUUUCA | UGAAAUCAGAAGCGCUCCA | [270-288] | — | — |
| Single Sp | 19 | CUGGAGCGCUUCUGAUUUC | GAAAUCAGAAGCGCUCCAG | [269-287] | — | — |
| Single Sp | 19 | UGUCUGGAGCGCUUCUGAU | AUCAGAAGCGCUCCAGACA | [266-284] | — | — |
| Single Sp | 19 | AUGUCUGGAGCGCUUCUGA | UCAGAAGCGCUCCAGACAU | [265-283] | — | — |
| Single Sp | 19 | CCACAUCACCUUUGUGGCU | AGCCACAAAGGUGAUGUGG | [173-191] | [1730-1743] | [246-264] |
| Single Sp | 19 | GACCACAUCACCUUUGUGG | CCACAAAGGUGAUGUGGUC | [171-189] | [1730-1741] | [244-262] |
| single Sp | 19 | CGACCACAUCACCUUUGUG | CACAAAGGUGAUGUGGUCG | [170-188] | [1730-1740] | [244-261] |
| Single Sp | 19 | GGAAGAGACAAUAAAGAUG | CAUCUUUAUUGUCUCUUCC | [2285-2303] | — | — |
| Single Sp | 19 | UGGGAAGAGACAAUAAAGA | UCUUUAUUGUCUCUUCCCA | [2283-2301] | — | — |
| Single Sp | 19 | UUUGGGAAGAGACAAUAAA | UUUAUUGUCUCUUCCCAAA | [2281-2299] | — | — |
| Single Sp | 19 | GUGUGCUUUGGGAAGAGAC | GUCUCUUCCCAAAGCACAC | [2275-2293] | — | — |
| Single Sp | 19 | GAAGCUCUGUUAGAAACAC | GUGUUUCUAACAGAGCUUC | [2255-2273] | — | — |
| Single Sp | 19 | UGAAGCUCUGUUAGAAACA | UGUUUCUAACAGAGCUUCA | [2254-2272] | — | — |
| Single Sp | 19 | UGAAUGAAGCUCUGUUAGA | UCUAACAGAGCUUCAUUCA | [2250-2268] | — | — |
| Single Sp | 19 | AAUGAAUGAAGCUCUGUUA | UAACAGAGCUUCAUUCAUU | [2248-2266] | — | — |
| Single Sp | 19 | GAAUGAAUGAAGCUCUGUU | AACAGAGCUUCAUUCAUUC | [2247-2265] | — | — |
| Single Sp | 19 | AGCCUGCCUGGGAAUGAAU | AUUCAUUCCCAGGCAGGCU | [2236-2254] | — | — |
| Single Sp | 19 | UAGCCUGCCUGGGAAUGAA | UUCAUUCCCAGGCAGGCUA | [2235-2253] | — | — |
| Single Sp | 19 | CUCCUUUCUACUCCUGGCU | AGCCAGGAGUAGAAAGGAG | [2184-2202] | — | — |
| Single Sp | 19 | CUGGCUCCUUUCUACUCCU | AGGAGUAGAAAGGAGCCAG | [2180-2198] | — | — |
| Single Sp | 19 | CAAGGACAUAUUCGUCACU | AGUGACGAAUAUGUCCUUG | [2102-2120] | — | — |
| Single Sp | 19 | CUACAAGGACAUAUUCGUC | GACGAAUAUGUCCUUGUAG | [2099-2117] | [3807-3818] | [2235-2246] |
| Single Sp | 19 | AAGGCUACAAGGACAUAUU | AAUAUGUCCUUGUAGCCUU | [2095-2113] | [3803-3818] | [2231-2246] |
| Single Sp | 19 | GAGAUCAAAGGCUACAAGG | CCUUGUAGCCUUUGAUCUC | [2088-2106] | [3796-3814] | [2224-2242] |
| Single Sp | 19 | ACGAGGUCAAGGAGAUCAA | UUGAUCUCCUUGACCUCGU | [2077-2095] | [3787-3803] | [2215-2231] |
| Single Sp | 19 | CCCUCCAAAUUCAACUGGA | UCCAGUUGAAUUUGGAGGG | [2005-2023] | — | [2148-2159] |
| Single Sp | 19 | ACACCCUCCAAAUUCAACU | AGUUGAAUUUGGAGGGUGU | [2002-2020] | — | — |
| Single Sp | 19 | GGACACACCCUCCAAAUUC | GAAUUUGGAGGGUGUGUCC | [1998-2016] | — | — |
| Single Sp | 19 | AUGGGCAGAUAGCAAAGGA | UCCUUUGCUAUCUGCCCAU | [1960-1978] | — | — |
| Single Sp | 19 | CUCAUCAAUGGGCAGAUAG | CUAUCUGCCCAUUGAUGAG | [1953-1971] | [3505-3521] | [2089-2105] |
| Single Sp | 19 | AGGAAGCGGCCUCAUCAAU | AUUGAUGAGGCCGCUUCCU | [1943-1961] | [3502-3513] | [2086-2097] |
| Single Sp | 19 | AAGGAAGCGGCCUCAUCAA | UUGAUGAGGCCGCUUCCUU | [1942-1960] | [3502-3512] | [2086-2096] |
| Single Sp | 19 | CUCACCAACACCUUAAUGG | CCAUUAAGGUGUUGGUGAG | [1893-1911] | [3445-3456] | [2029-2047] |
| Single Sp | 19 | CUUGUCUAUUGAGGUGUCU | AGACACCUCAAUAGACAAG | [1832-1850] | [3384-3400] | [1968-1984] |
| Single Sp | 19 | CCUCCCCACUUGUCUAUUG | CAAUAGACAAGUGGGGAGG | [18241842] | [3376-3394] | [1960-1978] |
| Single Sp | 19 | AGCCUCCCCACUUGUCUAU | AUAGACAAGUGGGGAGGCU | [1822-1840] | [3374-3392] | — |
| Single Sp | 19 | CUAAAAGAUAUCUGUCUG | CAGACAGAUAUCUUUUAGG | [1802-1820] | — | — |
| Single Sp | 19 | UGCUGGUCCUAAAAGAUAU | AUAUCUUUUAGGACCAGCA | [11795-1813] | [3349-3363] | [1933-1946] |

TABLE I
TGase7:21-mers

| | Oligo | | | human | mouse | rat | |
|---|---|---|---|---|---|---|---|
| Source | Length | Sense siRNA | AntiSense siRNA | 16445034 | 51712629 | 34857657 | |
| Single Sp | 21 | GGAACGACGUGGUGUAUGUGU | ACACAUACACCACGUCGUUCC | [652-672] | [2210-2224] | [802-818] | |
| Single Sp | 21 | CAAGGGUCAUGAAAGAUUCAU | AUGAAUCUUUCAUGACCCUUG | [515-535] | — | — | |
| Single Sp | 21 | GCAACGAGGUCAAGGAGAUCA | UGAUCUCCUUGACCUCGUUGC | [2074-2094] | [3787-3802] | [2215-2230] | |
| Single Sp | 21 | CCAUGCUGGUCCUAAAAGAUA | UAUCUUUUAGGACCAGCAUGG | [1792-1812] | [3349-3363] | [1933-1946] | |
| Single Sp | 21 | GGAGAGAGCUGUCUUCAUGAA | UUCAUGAAGACAGCUCUCUCC | [1367-1387] | — | — | |
| Single Sp | 21 | AUAGGAACUUGACCAUCGAUA | UAUCGAUGGUCAAGUUCCUAU | [934-954] | [2491-2504] | [1073-1088] | |
| Single Sp | 21 | ACAAGAGCCUGUAUCACUUAA | UUAAGUGAUACAGGCUCUUGU | [604-624] | — | [752-765] | |
| Single Sp | 21 | UUACAAGGGUCAUGAAAGAUU | AAUCUUUCAUGACCCUUGUAA | [512-532] | [2065-2075] | [585-598] | |
| Single Sp | 21 | CCAACUCUCUCCAAGUUUCCC | GGGAAACUUGGAGAGAGUUGG | [298-318] | [1851-1865] | [372-386] | |
| Single Sp | 21 | GGAUGAACCUGGACUUUGGGA | UCCCAAAGUCCAGGUUCAUCC | [1660-1680] | — | — | |
| Single Sp | 21 | GAACGCCGAUGAAGUCAUUUG | CAAAUGACUUCAUCGGCGUUC | [1205-1225] | — | — | |
| Single Sp | 21 | GGAAAGAUCUCCCACCAGGAU | AUCCUGGUGGGAGAUCUUUCC | [1045-1065] | — | — | |
| Single Sp | 21 | UGACCGAAAUGCCGAGAUGCU | AGCAUCUCGGCAUUUCGGUCA | [962-982] | — | — | |
| Single Sp | 21 | GUAAUGAGAUGCUUAGGUGUU | AACACCUAAGCAUCUCAUUAC | [870-890] | [2422-2442] | [1006-1026] | |
| Single Sp | 21 | UGAGUGCCAUGAUCAACAGCA | UGCUGUUGAUCAUGGCACUCA | [682-702] | [2239-2254] | [827-838] | |
| Single Sp | 21 | AUAGACAUCUGCUUUGAGAUC | GAUCUCAAAGCAGAUGUCUAU | [579-599] | [2137-2151] | [733-747] | |
| Single Sp | 21 | AAAUACUGCUGCAGGAGUAUA | UAUACUCCUGCAGCAGUAUUU | [469-489] | — | — | |
| Single Sp | 21 | AUAGAGAUCUCUCAGGGCCAA | UUGGCCCUGAGAGAUCUCUAU | [363-383] | [1916-1934] | — | |
| Single Sp | 21 | UCAAGGAGAUCAAAGGCUACA | UGUAGCCUUUGAUCUCCUUGA | [2083-2103] | [3791-3811] | [2219-2239] | |
| Single Sp | 21 | ACACCUUAAUGGUGGCUCUGA | UCAGAGCCACCAUUAAGGUGU | [1900-1920] | — | — | |
| Single Sp | 21 | AAAGAUAUCUGUCUGGAGCCU | AGGCUCCAGACAGAUAUCUUU | [1806-1826] | — | — | |
| Single Sp | 21 | AGAGAGCUGUCUUCAUGAAGG | CCUUCAUGAAGACAGCUCUCU | [1369-1389] | — | — | |
| Single Sp | 21 | GAAGUCAUUUGGCUCCUUGGO | CCCAAGGAGCCAAAUGACUUC | [1215-1235] | — | — | |
| Single Sp | 21 | AAAGACUGUUCCCAGCGGAAC | GUUCCGCUGGGAACAGUCUUU | [636-656] | — | — | |
| Single Sp | 21 | AAAGAUUCAUCACCUCCUGGC | GCCAGGAGGUGAUGAAUCUUU | [526-546] | — | — | |
| Single Sp | 21 | ACAUAUUCGUCACUGUGGCUG | CAGCCACAGUGACGAAUAUGU | [2107-2127] | — | — | |
| Single Sp | 21 | AAAUUCAACUGGACCUCUACC | GGUAGAGGUCCAGUUGAAUUU | [2011-2031] | — | [2148-2167] | |
| Single Sp | 21 | CUAUUGAGGUGUCUGAGAGGG | CCCUCUCAGACACCUCAAUAG | [1837-1857] | [3389-3409] | [1973-1984] | |
| Single Sp | 21 | CAAAGAGCUUCUUUGCCCUUC | GAAGGGCAAAGAAGCUCUUUG | [1413-1433] | — | — | |
| Single Sp | 21 | UCAUUUGGCUCCUUGGGGAUG | CAUCCCCAAGGAGCCAAAUGA | [1219-1239] | — | — | |
| Single Sp | 21 | UUAUGGCUUUGUUUACAAGGG | CCCUUGUAAACAAAGCCAUAA | [500-520] | — | [574-593] | |
| Single Sp | 21 | AAAUAGAGAUCUCUCAGGGCC | GGCCCUGAGAGAUCUCUAUUU | [361-381] | [1916-1933] | — | |
| Single Sp | 21 | GAGAUGCUGUCAACUCAGAAA | UUUCUGAGUUGACAGCAUCUC | [975-995] | — | — | |
| Single Sp | 21 | GGAACUUGACCAUCGAUACGU | ACGUAUCGAUGGUCAAGUUCC | [937-957] | [2491-2504] | [1073-1088] | |
| Single Sp | 21 | GCACAACGUGGAUAGGAACUU | AAGUUCCUAUCCACGUUGUGC | [923-943] | — | — | |
| Single Sp | 21 | CAACCCGUGUUGUUUCCAAUU | AAUUGGAAACAACACGGGUUG | [892-912] | — | — | |
| Single Sp | 21 | GGUGGUGAGUGCCAUGAUCAA | UUGAUCAUGGCACUCACCACC | [677-697] | [2229-2249] | — | |
| Single Sp | 21 | GAGCCUGUAUCACUUAAAGAA | UUCUUUAAGUGAUACAGGCUC | [608-628] | — | — | |
| Single Sp | 21 | GCUUUGAGAUCCUGAACAAGA | UCUUGUUCAGGAUCUCAAAGC | [589-609] | [2141-2161] | [737-757] | |
| Single Sp | 21 | GCGAGAUUAUGGCUUUGUUUA | UAAACAAAGCCAUAAUCUCGC | [494-514] | — | [574-587] | |
| Single Sp | 21 | CCAUUACACUCUGAAAAUAGA | UCUAUUUUCAGAGUGUAAUGG | [347-367] | — | — | |
| Single Sp | 21 | GCAGUUAUUGGCCAUUACACU | AGUGUAAUGGCCAAUAACUGC | [336-356] | — | — | |
| Single Sp | 21 | CUCCAAGUUUCCCUUUUCACA | UGUGAAAAGGGAAACUUGGAG | [306-326] | — | — | |
| Single Sp | 21 | GACUCCAACUCUCUCCAAGUU | AACUUGGAGAGAGUUGGAGUC | [294-314] | [1851-1865] | [372-386] | |
| Single Sp | 21 | CUUCUGAUUUCACCAUUGACU | AGUCAAUGGUGAAAUCAGAAG | [277-297] | — | [682-698] | |
| Single Sp | 21 | GGGAAUGAAUGAAGCUCUGUU | AACAGAGCUUCAUUCAUUCCC | [2245-2265] | — | — | |
| Single Sp | 21 | CCUUUCUACUCCUGGCUAUGU | ACAUAGCCAGGAGUAGAAAGG | [2186-2206] | — | — | |
| Single Sp | 21 | GAAGGAAGCGGCCUCAUCAAU | AUUGAUGAGGCCGCUUCCUUC | [1941-1961] | [3502-3513] | [2086-2097] | |
| Single Sp | 21 | CAAGCUAACGGACGAAAAGCU | AGCUUUUCGUCCGUUAGCUUG | [1727-1747] | — | — | |
| Single Sp | 21 | CUACAGCAAUUACAGAAACAA | UUGUUUCUGUAAUUGCUGUAG | [1709-1729] | — | [1845-1859] | |
| Single Sp | 21 | GAAGGCUUCUCGGAAAAUGCU | AGCAUUUUCCGAGAAGCCUUC | [1385-1405] | — | — | |
| Single Sp | 21 | GCAUCACCAGCUCCUACAAGU | ACUUGUAGGAGCUGGUGAUGC | [1327-1347] | — | — | |
| Single Sp | 21 | GGAAGGAGAUCAGCACUAAGA | UCUUAGUGCUGAUCUCCUUCC | [1282-1302] | — | — | |
| Single Sp | 21 | GAACUUCCACGUCUGGAAUGA | UCAUUCCAGACGUGGAAGUUC | [1010-1030] | [2562-2582] | — | |
| Single Sp | 21 | GACAAAAUAUGGAACUUCCAC | GUGGAAGUUCCAUAUUUUGUC | [999-1019] | [2551-2571] | [1135-1154] | |
| Single Sp | 21 | AGAAACGAGACAAAAUAUGGA | UCCAUAUUUUGUCUCGUUUCU | [991-1011] | — | — | |
| Single Sp | 21 | CCGAGAUGCUGUCAACUCAGA | UCUGAGUUGACAGCAUCUCGG | [973-993] | — | — | |
| Single Sp | 21 | GAAAUGCCGAGAUGCUGUCAA | UUGACAGCAUCUCGGCAUUUC | [967-987] | — | — | |
| Single Sp | 21 | GUACUAUGACCGAAAUGCCGA | UCGGCAUUUCGGUCAUAGUAC | [956-976] | — | [1093-1105] | |
| Single Sp | 21 | CCAUCGAUACGUACUAUGACC | GGUCAUAGUACGUAUCGAUGG | [946-966] | — | — | |
| Single Sp | 21 | CGCACAACGUGGAUAGGAACU | AGUUCCUAUCCACGUUGUGCG | [922-942] | — | — | |
| Single Sp | 21 | CGUGUUGUUUCCAAUUUCCGU | ACGGAAAUUGGAAACAACACG | [897-917] | [2455-2467] | [1039-1051] | |
| Single Sp | 21 | AACCCGUGUUGUUUCCAAUUU | AAAUUGGAAACAACACGGGUU | [893-913] | [2445-2465] | [1029-1049] | |
| Single Sp | 21 | UCCAACCCGUGUUGUUUCCAA | UUGGAAACAACACGGGUUGGA | [890-910] | — | — | |
| Single Sp | 21 | GCACCGUAAUGAGAUGCUUAG | CUAAGCAUCUCAUUACGGUGC | [865-885] | [2417-2437] | [1001-1021] | |
| Single Sp | 21 | CAGCAACGAUGACAAUGGCGU | ACGCCAUUGUCAUCGUUGCUG | [698-718] | — | — | |
| Single Sp | 21 | CAACAGCAACGAUGACAAUGG | CCAUUGUCAUCGUUGCUGUUG | [695-715] | — | — | |
| Single Sp | 21 | UGAUCAACAGCAACGAUGACA | UGUCAUCGUUGCUGUUGAUCA | [691-711] | [2243-2255] | [827-839] | |
| Single Sp | 21 | AGAACCCGGCCAAAGACUGUU | AACAGUCUUUGGCCGGGUUCU | [625-645] | — | — | |
| Single Sp | 21 | UGAACAAGAGCCUGUAUCACU | AGUGAUACAGGCUCUUGUUCA | [601-621] | [2153-2165] | [749-765] | |
| Single Sp | 21 | UCAUAGACAUCUGCUUUGAGA | UCUCAAAGCAGAUGUCUAUGA | [577-597] | [2137-2149] | [733-745] | |
| Single Sp | 21 | GGACAUCAUAGACAUCUGCUU | AAGCAGAUGUCUAUGAUGUCC | [572-592] | — | — | |
| Single Sp | 21 | GGAACAACAAGGAGCACCACA | UGUGGUGCUCCUUGUUGUUCC | [58-78] | [1610-1630] | [131-151] | |
| Single Sp | 21 | AAGAGGACAUCAUAGACAUCU | AGAUGUCUAUGAUGUCCUCUU | [568-588] | [2120-2130] | [716-726] | |
| Single Sp | 21 | CGGGCAGUUUGAAGAGGACAU | AUGUCCUCUUCAAACUGCCCG | [557-577] | [2110-2129] | [710-725] | |
| Single Sp | 21 | GGAACUACGGGCAGUUUGAAG | CUUCAAACUGCCCGUAGUUCC | [550-570] | [2102-2122] | [623-636] | |
| Single Sp | 21 | CGAGAUUAUGGCUUUGUUUAC | GUAAACAAAGCCAUAAUCUCG | [495-515] | — | [574-588] | |
| Single Sp | 21 | AGGAGUAUAUCAUGCGAGAUU | AAUCUCGCAUGAUAUACUCCU | [481-501] | [2034-2046] | [555-567] | |
| Single Sp | 21 | GCAGGAGUAUAUCAUGCGAGA | UCUCGCAUGAUAUACUCCUGC | [479-499] | [2034-2046] | [555-567] | |
| Single Sp | 21 | GUGAAAUACUGCUGCAGGAGU | ACUCCUGCAGCAGUAUUUCAC | [468-486] | — | — | |
| Single Sp | 21 | CCUGCCAAGUGAAAUACUGCU | AGCAGUAUUUCACUUGGCAGG | [458-478] | [2010-2023] | — | |
| Single Sp | 21 | ACGUCUACCUGCCAAGUGAAA | UUUCACUUGGCAGGUAGACGU | [451-471] | [2005-2023] | [526-536] | |
| Single Sp | 21 | UGGGAACUUUCAUCCUACUUU | AAAGUAGGAUGAAAGUUCCCA | [406-426] | — | — | |
| Single Sp | 21 | UUACCCGCUGGGAACUUUCAU | AUGAAAGUUCCCAGCGGGUAA | [398-418] | — | — | |
| Single Sp | 21 | UGACUUACCCGCUGGGAACUU | AAGUUCCCAGCGGGUAAGUCA | [394-414] | — | — | |
| Single Sp | 21 | CAAUGCAGUUAUUGGCCAUUA | UAAUGGCCAAUAACUGCAUUG | [332-352] | — | — | |
| Single Sp | 21 | CCAAUGCAGUUAUUGGCCAUU | AAUGGCCAAUAACUGCAUUGG | [331-351] | — | — | |
| Single Sp | 21 | UCACACCAGCCAAUGCAGUUA | UAACUGCAUUGGCUGGUGUGA | [322-342] | — | — | |
| Single Sp | 21 | CAACUCUCUCCAAGUUUCCCU | AGGGAAACUUGGAGAGAGUUG | [299-319] | [1851-1665] | [372-386] | |
| Single Sp | 21 | CUCCAACUCUCUCCAAGUUUC | GAAACUUGGAGAGAGUUGGAG | [296-316] | [1851-1865] | [372-386] | |
| Single Sp | 21 | GAGCGCUUCUGAUUUCACCAU | AUGGUGAAAUCAGAAGCGCUC | [272-292] | — | [682-693] | |
| Single Sp | 21 | GCCUGGGAAUGAAUGAAGCUC | GAGCUUCAUUCAUUCCCAGGC | [2241-2261] | — | — | |
| Single Sp | 21 | CUACUCCUGGCUAUGUCGUCU | AGACGACAUAGCCAGGAGUAG | [2191-2211] | — | — | |
| Single Sp | 21 | UCAAAGGCUACAAGGACAUAU | AUAUGUCCUUGUAGCCUUUGA | [2092-2112] | [3800-3818] | [2228-2246] | |
| Single Sp | 21 | GGAGAUCAAAGGCUACAAGGA | UCCUUGUAGCCUUUGAUCUCC | [2087-2107] | [3795-3815] | [2223-2243] | |
| Single Sp | 21 | CCUCCAAAUUCAACUGGACCU | AGGUCCAGUUGAAUUUGGAGG | [2006-2026] | — | [2148-2159] | |
| Single Sp | 21 | ACACACCCUCCAAAUUCAACU | AGUUGAAUUUGGAGGGUGUGU | [2000-2020] | — | — | |
| Single Sp | 21 | GACACACCCUCCAAAUUCAAC | GUUGAAUUUGGAGGGUGUGUC | [1999-2019] | — | | |
| Single Sp | 21 | UCAUCAAUGGGCAGAUAGCAA | UUGCUAUCUGCCCAUUGAUGA | [1954-1974] | [3506-3521] | [2090-21051 | |
| Single Sp | 21 | CCUUAAUGGUGGCUCUGAGCA | UGCUCAGAGCCACCAUUAAGG | [1903-1923] | — | — | |
| Single Sp | 21 | UCACCCUCACCAACACCUUAA | UUAAGGUGUUGGUGAGGGUGA | [1888-1908] | [3440-3456] | [2024-2040] | |
| Single Sp | 21 | CUCCCCACUUGUCUAUUGAGG | CCUCAAUAGACAAGUGGGGAG | [1825-1845] | [3377-3397] | [1961-1981] | |
| Single Sp | 21 | UGUCCAUGCUGGUCCUAAAAG | CUUUUAGGACCAGCAUGGACC | [1789-1809] | [3349-3361] | [1933-1945] | |
| Single Sp | 21 | GAAACAAGCUAACGGACGAAA | UUUCGUCCGUUAGCUUGUUUC | [1723-1743] | — | — | |
| Single Sp | 21 | CCAGAACGACCACAUCACCUU | AAGGUGAUGUGGUCGUUCUGG | [164-184] | — | — | |
| Single Sp | 21 | CCAAAGAGCUUCUUUGCCCUU | AAGGGCAAAGAAGCUCUUUGG | [1412-1432] | — | — | |
| Single Sp | 21 | CUGUCUUCAUGAAGGCUUCUC | GAGAAGCCUUCAUGAAGACAG | [1375-1395] | — | — | |
| Single Sp | 21 | GAGCUGUCUUCAUGAAGGCUU | AAGCCUUCAUGAAGACAGCUC | [1372-1392] | — | — | |
| Single Sp | 21 | UGAGGAGAGAGCUGUCUUCAU | AUGAAGACAGCUCUCUCCUCA | [1364-1384] | — | — | |
| Single Sp | 21 | CUACAAGUACCCAGAAGGAUC | GAUCCUUCUGGGUACUUGUAG | [1340-1360] | [2892-2912] | [1476-1496] | |
| Single Sp | 21 | CAACACCAGUUCCAUCGGGAA | UUCCCGAUGGAACUGGUGUUG | [1265-1285] | — | — | |
| Single Sp | 21 | AGGAAAUCCUGGCCCACAACA | UGUUGUGGGCCAGGAUUUCCU | [1249-12691 | [2801-2821] | [1385-1405] | |
| Single Sp | 21 | CUAUGACACCCCUUUUGUGUA | UACACAAAAGGGGUGUCAUAG | [1175-1195] | [2727-2747] | [1311-1328] | |
| Single Sp | 21 | AUGUCCACCUGGCCUAUGACA | UGUCAUAGGCCAGGUGGACAU | [1162-1182] | — | — | |
| Single Sp | 21 | AGAUCUCCCACCAGGAUACAA | UUGUAUCCUGGUGGGAGAUCU | [1049-1069] | — | — | |
| Single Sp | 21 | GAAAGAUCUCCCACCAGGAUA | UAUCCUGGUGGGAGAUCUUUC | [1046-1066] | — | — | |
| Single Sp | 21 | GGAUGAUCCGGAAAGAUCUCC | GGAGAUCUUUCCGGAUCAUCC | [1036-1056] | — | — | |
| Single Sp | 21 | GGAACUUCCACGUCUGGAAUG | CAUUCCAGACGUGGAAGUUCC | [1009-1029] | [2561-2581] | — | |
| Single Sp | 21 | UCAGAAACGAGACAAAAUAUG | CAUAUUUUGUCUCGUUUCUGA | [989-1009] | — | — | |
| Single Sp | 21 | AUGCCGAGAUGCUGUCAACUC | GAGUUGACAGCAUCUCGGCAU | [970-990] | — | — | |
| Single Sp | 21 | UACGUACUAUGACCGAAAUGC | GCAUUUCGGUCAUAGUACGUA | [953-973] | — | [1093-1105] | |
| Single Sp | 21 | ACUUGACCAUCGAUACGUACU | AGUACGUAUCGAUGGUCAAGU | [940-960] | [2492-2504] | [1076-1088] | |
| Single Sp | 21 | CAAUUUCCGUUCCGCGCACAA | UUGUGCGCGGAACGGAAAUUG | [908-928] | — | — | |
| Single Sp | 21 | UUAGGUGUUCCAACCCGUGUU | AACACGGGUUGGAACACCUAA | [882-902] | [2434-2450] | [1018-1034] | |
| Single Sp | 21 | CUUAGGUGUUCCAACCCGUGU | ACACGGGUUGGAACACCUAAG | [881-901] | [2433-2450] | [1017-1034] | |
| Single Sp | 21 | UUAUGUGCACCGUAAUGAGAU | AUCUCAUUACGGUGCACAUAA | [859-879] | — | — | |
| Single Sp | 21 | UCUGUUAUGUGCACCGUAAUG | CAUUACGGUGCACAUAACAGA | [855-875] | [2407-2427] | [991-1011] | |
| Single Sp | 21 | CUUCGCCUCUGUUAUGUGCAC | GUGCACAUAACAGAGGCGAAG | [848-868] | [2407-2420] | [988-1004] | |
| Single Sp | 21 | UCAACAGCAACGAUGACAAUG | PAUUGUCAUCGUUGCUGUUGA | [694-714] | — | — | |
| Single Sp | 21 | GUGCCAUGAUCAACAGCAACG | CGUUGCUGUUGAUCAUGGCAC | [685-7051 | [2239-2255] | [827-839] | |
| Single Sp | 21 | ACGACGUGGUGUAUGUGUGCA | UGCACACAUACACCACGUCGU | [655-675] | [2210-22271 | [803-823] | |
| Single Sp | 21 | CAAAGACUGUUCCCAGCGGAA | UUCCGCUGGGAACAGUCUUUG | [635-655] | — | — | |
| Single Sp | 21 | AAAGAACCCGGCCAAAGACUG | CAGUCUUUGGCCGGGUUCUUU | [623-643] | — | — | |
| Single Sp | 21 | UAAAGAACCCGGCCAAAGACU | AGUCUUUGGCCGGGUUCUUUA | [622-642] | — | — | |
| Single Sp | 21 | AGAUCCUGAACAAGAGCCUGU | ACAGGCUCUUGUUCAGGAUCU | [595-615] | [2147-2165] | [743-763] | |
| Single Sp | 21 | ACAUCUGCUUUGAGAUCCUGA | UCAGGAUCUCAAAGCAGAUGU | [583-603] | [2137-2155] | [733-747] | |
| Single 5p | 21 | ACAUCAUAGACAUCUGCUUUG | CAAAGCAGAUGUCUAUGAUGU | [574-594] | — | — | |
| Single Sp | 21 | AUGAAAGAUUCAUCACCUCCU | AGGAGGUGAUGAAUCUUUCAU | [523-543] | — | — | |
| Single Sp | 21 | GUAUAUCAUGCGAGAUUAUGG | CCAUAAUCUCGCAUGAUAUAC | [485-505] | — | — | |
| Single Sp | 21 | AGAGCUCCAGGAACAACAAGG | CCUUGUUGUUCCUGGAGCUCU | [49-69] | [1607-1621] | [128-142] | |
| Single Sp | 21 | CAAGUGAAAUACUGCUGCAGG | CCUGCAGCAGUAUUUCACUUG | [463-483] | — | — | |
| Single Sp | 21 | UCAUCCUACUUUUUAACCCUU | GGGUUAAAAAGUAGGAUGA | [415-435] | [1975-1985] | [496-506] | |
| Single Sp | 21 | UGCAGUUAUUGGCCAUUACAC | UGUAAUGGCCAAUAACUGCA | [335-355] | — | — | |
| Single Sp | 21 | ACCAGCCAAUGCAGUUAUUGG | CAAUAACUGCAUUGGCUGGU | [326-346] | — | — | |
| Single Sp | 21 | UUUCCCUUUUCACACCAGCCA | GGCUGGUGUGAAAAGGGAAA | [313-333] | — | — | |
| Single Sp | 21 | CCAAGUUUCCCUUUUCACACC | GUGUGAAAAGGGAAACUUGG | [308-328] | — | — | |
| Single Sp | 21 | UUGAGUCUGUCGACCUGCAGA | CUGCAGGUCGACAGACUCAA | [31-51] | — | — | |
| Single Sp | 21 | AGAUGGAUCAGGUGGCAACCU | GGUUGCCACCUGAUCCAUCU | [4-24] | — | — | |
| Single Sp | 21 | CCAUUGACUCCAACUCUCUCC | GAGAGAGUUGGAGUCAAUGG | [289-309] | [1851-1861] | [372-382] | |
| Single Sp | 21 | ACCAUUGACUCCAACUCUCUC | GAGAGUUGGAGUCAAUGGU | [288-308] | — | — | |
| Single Sp | 21 | UCACCAUUGACUCCAACUCUC | AGAGUUGGAGUCAAUGGUGA | [286-306] | — | — | |
| Single Sp | 21 | UUUCACCAUUGACUCCAACUC | GUUGGAGUCAAUGGUGAAA | [84-304] | — | — | |
| Single Sp | 21 | ACACCGUGUGCUUUGGGAAGA | CUUCCCAAAGCACACGGUGU | [2270-2290] | — | — | |
| Single Sp | 21 | GAAACACCGUGUGCUUUGGGA | CCCAAAGCACACGGUGUUUC | [2267-2287] | — | — | — |
| Single Sp | 21 | UUAGAAACACCGUGUGCUUUG | CAAAGCACACGGUGUUUCUAA | [2264-2284] | — | — | |
| Single Sp | 21 | AUGAAGCUCUGUUAGAAACAC | UGUUUCUAACAGAGCUUCAU | [2253-2273] | — | — | |
| Single Sp | 21 | GAAUGAAGCUCUGUUAGAAAC | UUUCUAACAGAGCUUCAUUC | [2251-2271] | — | — | |
| Single Sp | 21 | UUCUACUCCUGGCUAUGUCGU | CGACAUAGCCAGGAGUAGAA | [2189-2209] | — | — | |
| Single Sp | 21 | GGACAUAUUCGUCACUGUGGC | CCACAGUGACGAAUAUGUCC | [2105-2125] | — | — | |
| Single Sp | 21 | ACAAGGACAUAUUCGUCACUG | AGUGACGAAUAUGUCCUUGU | [2101-2121] | — | — | |
| Single Sp | 21 | AGAUCAAAGGCUACAAGGACA | GUCCUUGUAGCCUUUGAUCU | [2089-2109] | [3797-3817] | [2225-2245] | |
| Single Sp | 21 | UCAUCAGCAGCAACGAGGUCA | UGACCUCGUUGCUGCUGAUGA | [2065-2085] | [3773-3793] | [2201-2221] | |
| Single Sp | 21 | UCCAGGUUCUCAUCAGCAGCA | GCUGCUGAUGAGAACCUGGA | [2056-2076] | [3772-3784] | [2200-2212] | |
| Single Sp | 21 | AAAGGACCUUGGGACUCUGGU | CCAGAGUCCCAAGGUCCUUU | [1973-1993] | [3687-3699] | [2113-2127] | |
| Single Sp | 21 | AGAUAGCAAAGGACCUUGGGA | CCCAAGGUCCUUUGCUAUCU | [1966-1986] | — | — | |
| Single Sp | 21 | UCAAUGGGCAGAUAGCAAAGG | CUUUGCUAUCUGCCCAUUGA | [1957-1977] | [3509-3521] | [2093-2105] | |
| Single Sp | 21 | CCAACACCUUAAUGGUGGCUC | GAGCCACCAUUAAGGUGUUGG | [1897-1917] | — | — | |
| Single Sp | 21 | AUGUCACCCUCACCAACACCU | GGUGUUGGUGAGGGUGACAU | [1885-1905] | [3440-3456] | [2024-2040] | |
| Single Sp | 21 | CCACUUGUCUAUUGAGGUGUC | ACACCUCAAUAGACAAGUGG | [1829-1849] | [3381-3400] | [1967-1984] | |
| Single Sp | 21 | CCCCACUUGUCUAUUGAGGUG | ACCUCAAUAGACAAGUGGGG | [1827-1847] | [3379-3399] | [1967-1983] | |
| Single Sp | 21 | ACAUCACCUUUGUGGCUGAGA | CUCAGCCACAAAGGUGAUGU | [175-195] | [1730-1747] | [251-268] | |
| Single Sp | 21 | ACGAAAAGCUCAUCCGCGUGU | CACGCGGAUGAGCUUUUCGU | [1738-1758] | — | — | |
| Single Sp | 21 | ACAAGCUAACGGACGAAAAGC | CUUUUCGUCCGUUAGCUUGU | [1726-1746] | — | — | |
| Single Sp | 21 | ACGACCACAUCACCUUUGUGG | CACAAAGGUGAUGUGGUCGU | [169-189] | [1730-1741] | [244-262] | |
| Single Sp | 21 | AGAACGACCACAUCACCUUUG | AAAGGUGAUGUGGUCGUUCU | [166-186] | — | — | |
| Single Sp | 21 | AGAGCUUCUUUGCCCUUCCUG | AGGAAGGGCAAAGAAGCUCU | [1416-1436] | — | — | |
| Single Sp | 21 | AAAAUGCUGGGCCCCCAAAGA | CUUUGGGGGCCCAGCAUUUU | [1398-1418] | — | — | |
| Single Sp | 21 | AUGAAGGCUUCUCGGAAAAUG | AUUUUCCGAGAAGCCUUCAU | [1383-1403] | — | — | |
| Single Sp | 21 | AGAGCUGUCUUCAUGAAGGCU | GCCUUCAUGAAGACAGCUCU | [1371-1391] | — | — | |
| Single Sp | 21 | UCACCAGCUCCUACAAGUACC | GUACUUGUAGGAGCUGGUGA | [1330-13501 | [2882-2900] | [1466-1484] | |
| Single Sp | 21 | AUCACCAGCUCCUACAAGUAC | UACUUGUAGGAGCUGGUGAU | [1329-1349] | [2881-2900] | [1465-1484] | |
| Single Sp | 21 | UCAGCACUAAGAUGGUGGGGU | CCCCACCAUCUUAGUGCUGA | [1291-1311] | — | — | |
| Single Sp | 21 | CCGAUGAAGUCAUUUGGCUCC | GAGCCAAAUGACUUCAUCGG | [1210-1230] | — | — | |
| Single Sp | 21 | UGACACCCCUUUUGUGUAUGC | CAUACACAAAAGGGGUGUCA | [1178-1198] | [2734-2750] | [1318-1328] | |
| Single Sp | 21 | AAAGAUCUCCCACCAGGAUAC | UAUCCUGGUGGGAGAUCUUU | [1047-1067] | — | — | |
| Single Sp | 21 | AGUGCUGGAUGAUCCGGAAAG | UUUCCGGAUCAUCCAGCACU | [1030-1050] | [2582-2602] | [1166-1186] | |
| Single Sp | 21 | UGGAAUGAGUGCUGGAUGAUC | AUCAUCCAGCACUCAUUCCA | [1023-1043] | [2575-2595] | [1159-1179] | |
| Single Sp | 21 | UAUGGAACUUCCACGUCUGGA | CCAGACGUGGAAGUUCCAUA | [1006-1026] | [2560-2578] | [1144-1162] | |
| Single Sp | 21 | CGAGAUGCUGUCAACUCAGAA | UUCUGAGUUGACAGCAUCUCG | [974-994] | — | — | |
| Single Sp | 21 | CUUGACCAUCGAUACGUACUA | AGUACGUAUCGAUGGUCAAG | [941-961] | [2493-2513] | [1077-1097] | |
| Single Sp | 21 | GAACUUGACCAUCGAUACGUA | UACGUAUCGAUGGUCAAGUUC | [938-9581 | [2491-2504] | [1074-1088] | |
| Single Sp | 21 | GGUGUUCCAACCCGUGUUGUU | AACAACACGGGUUGGAACACC | [885-9051 | [2437-2450] | [1021-1034] | |
| Single Sp | 21 | CUGUUAUGUGCACCGUAAUGA | UCAUUACGGUGCACAUAACAG | [856-8761 | [2408-2428] | [992-1012] | |
| Single Sp | 21 | GGGUCUUCGCCUCUGUUAUGU | ACAUAACAGAGGCGAAGACCC | [844-8641 | — | [980-1000] | |
| Single Sp | 21 | CUGGGUCUUCGCCUCUGUUAU | AUAACAGAGGCGAAGACCCAG | [842-862] | — | [979-998] | |
| Single Sp | 21 | CAAGAGCCUGUAUCACUUAAA | UUUAAGUGAUACAGGCUCUUG | [605-625] | — | [753-765] | |
| Single Sp | 21 | GAACAAGAGCCUGUAUCACUU | AAGUGAUACAGGCUCUUGUUC | [602-6221 | [2154-21651 | [750-765] | |
| Single Sp | 21 | GCAGUUUGAAGAGGACAUCAU | AUGAUGUCCUCUUCAAACUGC | [560-580] | [2112-2130] | [710-726] | |
| Single Sp | 21 | GAACUACGGGCAGU0UGAAGA | JCUUCAAACUGCCCGUAGUUC | [551-571] | [2103-2123] | [624-636] | |
| Single Sp | 21 | GAGAUUAUGGCUUUGUUUACA | UGUAAACAAAGCCAUAAUCUC | [496-516] | — | [574-589] | |
| Single Sp | 21 | CAUGCGAGAUUAUGGCUUUGU | ACAAAGCCAUAAUCUCGCAUG | [491-511] | — | [574-584] | |
| Single Sp | 21 | CUACCUGCCAAGUGAAAUACU | AGUAUUUCACUUGGCAGGUAG | [455-475] | [2007-2023] | — | |
| Single Sp | 21 | GUCUACCUGCCAAGUGAAAUA | AAUUUCACUUGGCAGGUAGAC | [453-473] | [2005-2023] | [526-536] | |
| Single Sp | 21 | CGUCUACCUGCCAAGUGAAAU | AUUUCACUUGGCAGGUAGACG | [452-472] | [2005-2023] | [526-536] | |
| Single Sp | 21 | OGAACUUUCAUCCUACUUUUU | AAAAAGUAGGAUGAAAGUUCC | [408-428] | — | — | |
| Single Sp | 21 | CUGGGAACUUUCAUCCUACUU | AAGUAGGAUGAAAGUUCCCAG | [405-425] | — | — | |
| Single Sp | 21 | CCGCUGGGAACUUUCAUCCUA | UAGGAUGAAAGUUCCCAGCGG | [402-422] | — | — | |
| Single Sp | 21 | CCAAGGUCACAGUGUGACUUA | AAAGUCACACUGUGACCUUGG | [380-400] | — | — | |
| Single Sp | 21 | 0GCCAUUACACUCUGAAAAUA | UAUUUUCAGAGUGUAAUGGCC | [345-365] | — | — | |
| Single Sp | 21 | CUCUCCAAGUUUCCCUUUUCA | UGAAAAGGGAAACUUGGAGAG | [304-324] | — | — | |
| Single Sp | 21 | UUCUCUCCAAGUUUCCCUUUU | AAAAGGGAAACUUGGAGAGAG | [302-322] | [854-1865] | [375-395] | |
| Single Sp | 21 | CGCUUCUGAUUUCACCAUUGA | UCAAUGGUGAAAUCAGAAGCG | [275-295] | — | [682-698] | |
| Single Sp | 21 | GAAGAGACAAUAAAGAUGUCU | AGACAUCUUUAUUGUCUCUUC | [2286-2306] | — | — | |
| Single Sp | 21 | GUGUGCUUUGGGAAGAGACAA | UUGUCUCUUCCCAAAGCACAC | [2275-2295] | — | — | |
| Single Sp | 21 | GCCUGCCUGGGAAUGAAUGAA | UUCAUUCAUUCCCAGGCAGGC | [2237-2257] | — | — | |
| Single Sp | 21 | CAAGGAGAUCAAAGGCUACAA | UUGUAGCCUUUGAUCUCCUUG | [2084-2104] | [3792-3812] | [2220-2240] | |
| Single Sp | 21 | GGACACACCCUCCAAAUUCAA | UUGAAUUUGGAGGGUGUGUCC | [1998-2018] | — | — | |
| Single Sp | 21 | CGGACACACCCUCCAAAUUCA | UGAAUUUGGAGGGUGUGUCCG | [1997-2017] | — | — | |
| Single Sp | 21 | CUCAUCAAUGGGCAGAUAGCA | UGCUAUCUGCCCAUUGAUGAG | [1953-1973] | [3505-3521] | [2089-2105] | |
| Single Sp | 21 | GUCACCCUCACCAACACCUUA | UAAGGUGUUGGUGAGGGUGAC | [1887-1907] | [3440-3456] | [2024-2040] | |
| Single Sp | 21 | pAUGCUGGUCCUAAAAGAUAU | AUAUCUUUUAGGACCAGCAUG | [1793-1813] | [3349-3363] | [1933-1946] | |
| Single Sp | 21 | GUCCAUGCUGGUCCUAAAAGA | UCUUUUAGGACCAGCAUGGAC | [1790-1810] | [3349-3362] | [1933-1946] | |
| Single Sp | 21 | GCAAUUACAGAAACAAGCUAA | UUAGCUUGUUUCUGUAAUUGC | [1714-1734] | — | — | |
| Single Sp | 21 | CUGCCCUACAGCAAUUACAGA | UCUGUAAUUGCUGUAGGGCAG | [1704-1724] | [3256-3275] | [1840-1859] | |
| Single Sp | 21 | GAACGACCACAUCACCUUUGU | ACAAAGGUGAUGUGGUCGUUC | [167-187] | — | — | |
| Single Sp | 21 | CAUGAAGGCUUCUCGGAAAAU | AUUUUCCGAGAAGCCUUCAUG | [1382-1402] | — | — | |
| Single Sp | 21 | CCUACAAGUACCCAGAAGGAU | AUCCUUCUGGGUACUUGUAGG | [1339-1359] | — | — | |
| Single Sp | 21 | GAAGGAGAUCAGCACUAAGAU | AUCUUAGUGCUGAUCUCCUUC | [1283-1303] | — | — | |
| Single Sp | 21 | UUUCCAUCGGGAAGGAGAUCA | UGAUCUCCUUCCCGAUGGAAC | [1273-1293] | — | — | |
| Single Sp | 21 | GGUGAACGCCGAUGAAGUCAU | AUGACUUCAUCGGCGUUCACC | [1202-1222] | — | — | |
| Single Sp | 21 | CCUAUGACACCCCUUUUGUGU | ACACAAAAGGGGUGUCAUAGG | [1174-1194] | [2726-2746] | [1310-1328] | |
| Single Sp | 21 | CGAGACAAAAUAUGGAACUUC | GAAGUUCCAUAUUUUGUCUCG | [996-1016] | — | — | |
| Single Sp | 21 | GAAACGAGACAAAAUAUGGAA | UUCCAUAUUUUGUCUCGUUUC | [992-1012] | — | — | |
| Single Sp | 21 | AACUCAGAAACGAGACAAAAO | AUUUUGUCUCGUUUCUGAGUU | [986-10061 | — | — | |
| Single Sp | 21 | UCAACUCAGAAACGAGACAAA | UUUGUCUCGUUUCUGAGUUGA | [984-1004] | — | — | |
| Single Sp | 21 | GCUGUCAACUCAGAAACGAGA | UCUCGUUUCUGAGUUGACAGC | [980-1000] | — | — | |
| Single Sp | 21 | AUGCUGUCAACUCAGAAACGA | UCGUUUCUGAGUUGACAGCAU | [978-998] | — | — | |
| Single Sp | 21 | CGAAAUGCCGAGAUGCUGUCA | UGACAGCAUCUCGGCAUUUCG | [966-986] | — | — | |
| Single Sp | 21 | CGAUACGUACUAUGACCGAAA | UUUCGGUCAUAGUACGUAUCG | [950-970] | — | [1093-1105] | |
| Single Sp | 21 | GAUAGGAACUUGACCAUCGAU | AUCGAUGGUCAAGUUCCUAUC | [933-953] | [2491-2504] | [1073-1088] | |
| Single Sp | 21 | CAACGUGGAUAGGAACUUGAC | GUCAAGUUCCUAUCCACGUUG | [926-946] | — | — | |
| Single Sp | 21 | GUGUUGUUUCCAAUUUCCGUU | AACGGAAAUUGGAAACAACAC | [898-918] | [2455-2467] | [1039-1051] | |
| Single Sp | 21 | CCGUGUUGUUUCCAAUUUCCG | CGGAAAUUGGAAACAACACGG | [896-916] | [2455-2467] | [1039-1051] | |
| Single Sp | 21 | CCCGUGUUGUUUCCAAUUUCC | GGAAAUUGGAAACAACACGGG | [895-915] | [2455-2467] | [1039-1051] | |
| Single Sp | 21 | 3AGAUGCUUAGGUGUUCCAAC | bUUGGAACACCUAAGCAUCUC | [875-895] | [2427-2447] | [1011-1031] | |
| Single Sp | 21 | UGAGAUGCUUAGGUGUUCCAA | bUGGAACACCUAAGCAUCUCA | [874-894] | [2426-2446] | [1010-1030] | |
| Single Sp | 21 | ACCGUAAUGAGAUGCUUAGGU | ACCUAAGCAUCUCAUUACGGU | [867-887] | [2422-2439] | [1006-1023] | |
| Single Sp | 21 | CCUCUGUUAUGUGCACCGUAA | UUACGGUGCACAUAACAGAGG | [853-873] | [2407-2425] | [1989-1009] | |
| Single Sp | 21 | GUCUUCGCCUCUGUUAUGUGC | GCACAUAACAGAGGCGAAGAC | [846-866] | [2398-2415] | [982-1002] | |
| Single Sp | 21 | CAACGAUGACAAUGGCGUGCU | AGCACGCCAUUGUCAUCGUUG | [701-721] | — | — | |
| Single Sp | 21 | CCAUGAUCAACAGCAACGAUG | CAUCGUUGCUGUUGAUCAUGG | [688-708] | [2240-2255] | [827-839] | |
| Single Sp | 21 | UGGUGAGUGCCAUGAUCAACA | UGUUGAUCAUGGCACUCACCA | [679-699] | [2239-2251] | — | |
| Single Sp | 21 | GUGGUGAGUGCCAUGAUCAAC | GUUGAUCAUGGCACUCACCAC | [678-698] | [2230-2250] | — | |
| Single Sp | 21 | GAACGACGUGGUGUAUGUGUG | CACACAUACACCACGUCGUUC | [653-673] | [2210-2225] | [802-818] | |
| Single Sp | 21 | GCCUGUAUCACUUAAAGAACC | GGUUCUUUAAGUGAUACAGGC | [610-630] | — | — | |
| Single Sp | 21 | AGAGCCUGUAUCACUUAAAGA | UCUUUAAGUGAUACAGGCUCU | [607-627] | — | [755-765] | |
| Single Sp | 21 | CUGAACAAGAGCCUGUAUCAC | GUGAUACAGGCUCUUGUUCAG | [600-620] | [2152-2165] | [749-765] | |
| Single Sp | 21 | UCUGCUUUGAGAUCCUGAACA | UGUUCAGGAUCUCAAAGCAGA | [586-606] | [2138-2158] | [734-754] | |
| Single Sp | 21 | CAUCUGCUUUGAGAUCCUGAA | UUCAGGAUCUCAAAGCAGAUG | [584-6041 | [2137-2156] | [733-7521 | |
| Single Sp | 21 | GAAGAGGACAUCAUAGACAUC | GAUGUCUAUGAUGUCCUCUUC | [567-5871 | [2119-2130] | [715-726] | |
| Single Sp | 21 | UGAAGAGGACAUCAUAGACAU | AUGUCUAUGAUGUCCUCUUCA | [566-586] | [2118-2130] | [714-726] | |
| Single Sp | 21 | UUGAAGAGGACAUCAUAGACA | UGUCUAUGAUGUCCUCUUCAA | [565-585] | [2117-21301 | [713-726] | |
| Single Sp | 21 | GUUUGAAGAGGACAUCAUAGA | UCUAUGAUGUCCUCUUCAAAC | [563-583] | [2115-2130] | [711-726] | |
| Single Sp | 21 | CUACGGGCAGUUUGAAGAGGA | UCCUCUUCAAACUGCCCGUAG | [554-574] | [2110-2126] | [710-722] | |
| Single Sp | 21 | UGGAACUACGGGCAGUUUGAA | UUCAAACUGCCCGUAGUUCCA | [549-569] | [2101-2121] | [622-636] | |
| Single Sp | 21 | GGUCAUGAAAGAUUCAUCACC | GGUGAUGAAUCUUUCAUGACC | [519-539] | — | — | |
| Single Sp | 21 | ACAAGGGUCAUGAAAGAUUCA | UGAAUCUUUCAUGACCCUUGU | [514-534] | — | — | |
| Single Sp | 21 | GUUUACAAGGGUCAUGAAAGA | UCUUUCAUGACCCUUGUAAAC | [510-530] | [2065-2075] | [583-596] | |
| Single Sp | 21 | GCUUUGUUUACAAGGGUCAUG | CAUGACCCUUGUAAACAAAGC | [505-525] | [2065-2075] | [578-596] | |
| Single Sp | 21 | GGCUUUGUUUACAAGGGUCAU | AUGACCCUUGUAAACAAAGCC | [504-524] | [2056-20751 | [577-596] | |
| Single Sp | 21 | CUGCAGGAGUAUAUCAUGCGA | UCGCAUGAUAUACUCCUGCAG | [477-497] | [2029-20461 | [550-567] | |
| Single Sp | 21 | UACUGCUGCAGGAGUAUAUCA | UGAUAUACUCCUGCAGCAGUA | [472-492] | [2029-2044] | [546-565] | |
| Single Sp | 21 | CAGAGCUCCAGGAACAACAAG | CUUGUUGUUCCUGGAGCUCUG | [48-68] | [1607-1620] | [128-1411 | |
| Single Sp | 21 | GAAAUACUGCUGCAGGAGUAU | AUACUCCUGCAGCAGUAUUUC | [468-488] | — | — | |
| Single Sp | 21 | CUACUUUUUAACCCUUGGAGU | ACUCCAAGGGUUAAAAAGUAG | [420-440] | [1975-1992] | [496-513] | |
| Single Sp | 21 | CCUACUUUUUAACCCUUGGAG | CUCCAAGGGUUAAAAAGUAGG | [419-439] | [1975-1991] | [496-512] | |
| Single Sp | 21 | GCUGGGAACUUUCAUCCUACU | AGUAGGAUGAAAGUUCCCAGC | [404-424] | — | — | |
| Single Sp | 21 | CUUACCCGCUGGGAACUUUCA | UGAAAGUUCCCAGCGGGUAAG | [397-417] | — | — | |
| Single Sp | 21 | CAAGGUCACAGUGUGACUUAC | GUAAGUCACACUGUGACCUUG | [381-401] | [1943-1953] | — | |
| Single Sp | 21 | CUCUGAAAAUAGAGAUCUCUC | GAGAGAUCUCUAUUUUCAGAG | [355-375] | [1916-1927] | — | |
| Single Sp | 21 | ACACUCUGAAAAUAGAGAUCU | AGAUCUCUAUUUUCAGAGUGU | [352-372] | — | — | |
| Single Sp | 21 | UUGGCCAUUACACUCUGAAAA | UUUUCAGAGUGUAAUGGCCAA | [343-363] | — | — | |
| Single Sp | 21 | AUUGGCCAUUACACUCUGAAA | UUUCAGAGUGUAAUGGCCAAU | [342-362] | — | — | |
| Single Sp | 21 | CAGUUAUUGGCCAUUACACUC | GAGUGUAAUGGCCAAUAACUG | [337-357] | — | — | |
| Single Sp | 21 | CCAGCCAAUGCAGUUAUUGGC | GCCAAUAACUGCAUUGGCUGG | [327-347] | — | — | |
| Single Sp | 21 | CCUUUUCACACCAGCCAAUGC | GCAUUGGCUGGUGUGAAAAGG | [317-337] | — | — | |
| Single Sp | 21 | CAAGUUUCCCUUUUCACACCA | UGGUGUGAAAAGGGAAACUUG | [309-329] | — | —— | |
| Single Sp | 21 | CUGAUUUCACCAUUGACUCCA | UGGAGUCAAUGGUGAAAUCAG | [280-300] | — | [683-698] | |
| Single Sp | 21 | GCUUCUGAUUUCACCAUUGAG | GUCAAUGGUGAAAUCAGAAGC | [276-295] | — | [682-698] | |
| Single Sp | 21 | GGAGCGCUUCUGAUUUCACCA | UGGUGAAAUCAGAAGCGCUCC | [271-291] | — | [682-693] | |
| Single Sp | 21 | CUGGAGCGCUUCUGAUUUCAC | GUGAAAUCAGAAGCGCUCCAG | [269-289] | — | [682-692] | |
| Single Sp | 21 | GUCUGGAGCGCUUCUGAUUUC | GAAAUCAGAAGCGCUCCAGAC | [267-287] | — | — | |
| Single Sp | 21 | UGUCUGGAGCGCUUCUGAUUU | AAAUCAGAAGCGCUCCAGACA | [266-286] | — | — | |
| Single Sp | 21 | AUGUCUGGAGCGCUUCUGAUU | AAUCAGAAGCGCUCCAGACAU | [265-285] | — | — | |
| Single Sp | 21 | GAAUGUCUGGAGCGCUUCUGA | UCAGAAGCGCUCCAGACAUUC | [263-283] | — | — | |
| Single Sp | 21 | GGAAGAGACAAUAAAGAUGUC | GACAUCUUUAUUGUCUCUUCC | [2285-2305] | — | — | |
| Single Sp | 21 | UUUGGGAAGAGACAAUAAAGA | UCUUUAUUGUCUCUUCCCAAA | [2281-2301] | — | — | |
| Single Sp | 21 | UGUGCUUUGGGAAGAGACAAU | AUUGUCUCUUCCCAAAGCACA | [2276-2296] | — | — | |
| Single Sp | 21 | CGUGUGCUUUGGGAAGAGACA | UGUCUCUUCCCAAAGCACACG | [2274-2294] | — | — | |
| Single Sp | 21 | CUGUUAGAAACACCGUGUGCU | AGCACACGGUGUUUCUAACAG | [2261-2281] | — | — | |
| Single Sp | 21 | UGAAUGAAGCUCUGUUAGAAA | UUUCUAACAGAGCUUCAUUCA | [2250-2270] | — | — | |
| Single Sp | 21 | AUGAAUGAAGCUCUGUUAGAA | UUCUAACAGAGCUUCAUUCAU | [2249-2269] | — | — | |
| Single Sp | 21 | AAUGAAUGAAGCUCUGUUAGA | UCUAACAGAGCUUCAUUCAUU | [2248-2268] | — | — | |
| Single Sp | 21 | AGCCUGCCUGGGAAUGAAUGA | UCAUUCAUUCCCAGGCAGGCU | [2236-2256] | — | — | |
| Single Sp | 21 | CUCCUUUCUACUCCUGGCUAU | AUAGCCAGGAGUAGAAAGGAG | [2184-2204] | — | — | |
| Single Sp | 21 | pCUCCUUUCUACUCCUGGCUA | UAGCCAGGAGUAGAAAGGAGC | [2183-2203] | — | — | |
| Single Sp | 21 | CAAGGACAUAUUCGUCACUGU | ACAGUGACGAAUAUGUCCUUG | [2102-2122] | — | — | |
| Single Sp | 21 | GCUACAAGGACAUAUUCGUCA | UGACGAAUAUGUCCUUGUAGC | [2098-2118] | [3806-3818] | [2234-2246] | |
| Single Sp | 21 | AGGCUACAAGGACAUAUUCGU | ACGAAUAUGUCCUUGUAGCCU | [2096-2116] | [3804-38181 | [2232-2246] | |
| Single Sp | 21 | AUCAAAGGCUACAAGGACAUA | UAUGUCCUUGUAGCCUUUGAU | [2091-2111] | [3799-3818] | [2227-2246] | |
| Single Sp | 21 | GAGAUCAAAGGCUACAAGGAC | GUCCUUGUAGCCUUUGAUCUC | [2088-2108] | [3796-3816] | [7774-2244] | |
| Single Sp | 21 | CGAGGUCAAGGAGAUCAAAGG | CCUUUGAUCUCCUUGACCUCG | [2078-2098] | [3787-3808] | [2215-2234] | |
| Single Sp | 21 | CAGCAACGAGGUCAAGGAGAU | AUCUCCUUGACCUCGUUGCUG | [2072-2092] | [3787-3800] | [2215-2228] | |
| Single Sp | 21 | GUUCUCAUCAGCAGCAACGAG | CUCGUUGCUGCUGAUGAGAAC | [2061-2081] | [3772-3784] | [2200-2212] | |
| Single Sp | 21 | CCAGGUUCUCAUCAGCAGCAA | UUGCUGCUGAUGAGAACCUGG | [2057-2077] | [3772-3784] | [2200-2212] | |
| Single Sp | 21 | CCAAAUUCAACUGGACCUCUA | UAGAGGUCCAGUUGAAUUUGG | [2009-2029] | — | [2148-2165] | |
| Single Sp | 21 | GGGCAGAUAGCAAAGGACCUU | AAGGUCCUUUGCUAUCUGCCC | [1962-1982] | — | — | |
| Single Sp | 21 | CAAUGGGCAGAUAGCAAAGGA | UCCUUUGCUAUCUGCCCAUUG | [1958-1978] | [3510-3521] | [2094-2105] | |
| Single Sp | 21 | CCUCAUCAAUGGGCAGAUAGC | GCUAUCUGCCCAUUGAUGAGG | [1952-1972] | [3504-3521] | [2088-2105] | |
| Single Sp | 21 | CUCACCAACACCUUAAUGGUG | CACCAUUAAGGUGUUGGUGAG | [1893-1913] | [3445-3465] | [2029-2049] | |
| Single Sp | 21 | CCCUCACCAACACCUUAAUGG | CCAUUAAGGUGUUGGUGAGGG | [1891-1911] | [3443-3456] | [2027-2047] | |
| Single Sp | 21 | CUUGUCUAUUGAGGUGUCUGA | UCAGACACCUCAAUAGACAAG | [1832-18521 | [3384-3400] | [1968-1984] | |
| Single Sp | 21 | CCUCCCCACUUGUCUAUUGAG | CUCAAUAGACAAGUGGGGAGG | [1824-1844] | [3376-3396] | [1960-1980] | |
| Single Sp | 21 | CCUAAAAGAUAUCUGUCUGGA | UCCAGACAGAUAUCUUUUAGG | [1802-18221 | — | — | |
| Single Sp | 21 | GGUCCUAAAAGAUAUCUGUCU | AGACAGAUAUCUUUUAGGACC | [1799-1819] | [3351-3363] | [1935-1946] | |
| Single Sp | 21 | UGCUGGUCCUAAAAGAUAUCU | AGAUAUCUUUUAGGACCAGCA | [1795-1815] | [3349-3363] | [1933-1946] | |
| Single Sp | 21 | CAUCACCUUUGUGGCUGAGAC | GUCUCAGCCACAAAGGUGAUG | [176-196] | [1730-1748] | [251-269] | |
| Single Sp | 21 | GCUAACGGACGAAAAGCUCAU | AUGAGCUUUUCGUCCGUUAGC | [1730-1750] | — | — | |
| Single Sp | 21 | CAGAAACAAGCUAACGGACGA | UCGUCCGUUAGCUUGUUUCUG | [1721-1741] | — | — | |
| Single Sp | 21 | CCACAUCACCUUUGUGGCUGA | UCAGCCACAAAGGUGAUGUGG | [173-193] | [1730-1745] | [246-266] | |
| Single Sp | 21 | AGCAAUUACAGAAACAAGCUA | UAGCUUGUUUCUGUAAUUGCU | [1713-1733] | — | [1849-1869] | |
| Single Sp | 21 | UGCCCUACAGCAAUUACAGAA | UUCUGUAAUUGCUGUAGGGCA | [1705-1725] | [3257-3275] | [1841-1859] | |
| Single Sp | 21 | CUCCUGCCCUACAGCAAUUAC | GUAAUUGCUGUAGGGCAGGAG | [1701-1721] | [3256-3273] | [1840-1857] | |
| Single Sp | 21 | GACCACAUCACCUUUGUGGCU | AGCCACAAAGGUGAUGUGGUC | [171-191] | [1730-1743] | [244-264] | |
| Single Sp | 21 | UCCUCCUGGCCUACAGCAAUU | AAUUGCUGUAGGGCAGGAGGA | [1699-1719] | [3251-3268] | [1835-1855] | |
| Single Sp | 21 | CUGGACUUUGGGAAGGAGACA | UGUCUCCUUCCCAAAGUCCAG | [1668-1688] | — | — | |
| Single Sp | 21 | GAACCUGGACUUUGGGAAGGA | UCCUUCCCAAAGUCCAGGUUC | [1664-1684] | — | — | |
| Single Sp | 21 | GAUGAACCUGGACUUUGGGAA | UUCCCAAAGUCCAGGUUCAUC | [1661-1681] | — | — | |
| Single Sp | 21 | AGUCCCAGAACGACCACAUCA | UGAUGUGGUCGUUCUGGGACU | [160-180] | — | — | |
| Single Sp | 21 | CUUCUUUGCCCUUCCUGGAUC | GAUCCAGGAAGGGCAAAGAAG | [1420-1440] | — | — | |
| Single Sp | 21 | GCUUCUUUGCCCUUCCUGGAU | AUCCAGGAAGGGCAAAGAAGC | [1419-1439] | — | — | |
| Single Sp | 21 | UCAUGAAGGCUUCUCGGAAAA | UUUUCCGAGAAGCCUUCAUGA | [1381-1401] | — | — | |
| Single Sp | 21 | CUUCAUGAAGGCUUCUCGGAA | UUCCGAGAAGCCUUCAUGAAG | [1379-1399] | — | — | |
| Single Sp | 21 | GCUCCUACAAGUACCCAGAAG | CUUCUGGGUACUUGUAGGAGC | [1336-1356] | [2890-2908] | 1474-1494 | |
| Single Sp | 21 | GAGAUCAGCACUAAGAUGGUG | CACCAUCUUAGUGCUGAUCUC | [1287-1307] | — | | |
| Single Sp | 21 | GGAGAUCAGCACUAAGAUGGU | ACCAUCUUAGUGCUGAUCUCC | [1286-1306] | — | — | |
| Single Sp | 21 | CGAUGAAGUCAUUUGGCUCCU | AGGAGCCAAAUGACUUCAUCG | [1211-1231] | — | — | |
| Single Sp | 21 | CGCCGAUGAAGUCAUUUGGCU | AGCCAAAUGACUUCAUCGGCG | [1208-1228] | — | — | |
| Single Sp | 21 | UGAACGCCGAUGAAGUCAUUU | AAAUGACUUCAUCGGCGUUCA | [1204-1224] | — | — | |
| Single Sp | 21 | AGGUGAACGCCGAUGAAGUCA | UGACUUCAUCGGCGUUCACCU | [1201-1221] | — | — | |
| Single Sp | 21 | CACCCCUUUUGUGUAUGCCGA | UCGGCAUACACAAAAGGGGUG | [1181-1201] | [2734-2750] | [1318-1328] | |
| Single Sp | 21 | UAUGACACCCCUUUUGUGUAU | AUACACAAAAGGGGUGUCAUA | [1176-1196] | [2728-2748] | [1312-1328] | |
| Single Sp | 21 | GCCUAUGACACCCCUUUUGUG | CACAAAAGGGGUGUCAUAGGC | [1173-1193] | [2726-2745] | [1310-1328] | |
| Single Sp | 21 | GCUGGAUGAUCCGGAAAGAUC | GAUCUUUCCGGAUCAUCCAGC | [1033-1053] | [2585-2603] | [1169-1187] | |
| Single Sp | 21 | 0GAAUGAGUGCUGGAUGAUCC | GGAUCAUCCAGCACUCAUUCC | [1024-1044] | [2576-2595] | [1160-1179] | |
| Single Sp | 21 | CGUCUGGAAUGAGUGCUGGAU | AUCCAGCACUCAUUCCAGACG | [1019-1039] | [2571-2591] | [1158-1175] | |
| Single Sp | 21 | ACUUCCACGUCUGGAAUGAGU | ACUCAUUCCAGACGUGGAAGU | [1012-1032] | [2564-2584] | — | |
| Single Sp | 21 | AAACGAGACAAAAUAUGGAAC | GUUCCAUAUUUUGUCUCGUUU | [993-1013] | — | — | |
| Single Sp | 21 | UGUCAACUCAGAAACGAGACA | UGUCUCGUUUCUGAGUUGACA | [982-1002] | — | — | |
| Single Sp | 21 | AGAUGCUGUCAACUCAGAAAC | GUUUCUGAGUUGACAGCAUCU | [976-996] | — | — | |
| Single Sp | 21 | ACCGAAAUGCCGAGAUGCUGU | ACAGCAUCUCGGCAUUUCGGU | [964-984] | — | — | |
| Single Sp | 21 | CUAUGACCGAAAUGCCGAGAU | AUCUCGGCAUUUCGGUCAUAG | [959-979] | — | [1095-1105] | |
| Single Sp | 21 | AUACGUACUAUGACCGAAAUG | CAUUUCGGUCAUAGUACGUAU | [952-972] | — | [1095-1105] | |
| Single Sp | 21 | UCGAUACGUACUAUGACCGAA | UUCGGUCAUAGUACGUAUCGA | [949-969] | — | [1093-1105] | |
| Single Sp | 21 | AUCGAUACGUACUAUGACCGA | UCOGUCAUAGUACGUAUCGAU | [948-968] | — | [1084-1104] | |
| Single Sp | 21 | ACCAUCGAUACGUACUAUGAC | GUCAUAGUACGUAUCGAUGGU | [945-965] | — | — | |
| Single Sp | 21 | UGACCAUCGAUACGUACUAUG | CAUAGUACGUAUCGAUGGUCA | [943-963] | — | — | |
| Single Sp | 21 | AGGAACUUGACCAUCGAUACG | CGUAUCGAUGGUCAAGUUCCU | [936-956] | [2491-2504] | [1073-1088] | |
| Single Sp | 21 | ACGUGGAUAGGAACUUGACCA | UGGUCAAGUUCCUAUCCACGU | [928-948] | — | [1073-1084] | |
| Single Sp | 21 | AACGUGGAUAGGAACUUGACC | GGUCAAGUUCCUAUCCACGUU | [927-947] | — | [1073-1083] | |
| Single Sp | 21 | UUCCAAUUUCCGUUCCGCGCA | UGCGCGGAACGGAAAUUGGAA | [905-925] | [2457-2467] | [1041-1051] | |
| Single Sp | 21 | UGUUCCAACCCGUGUUGUUUC | GAAACAACACGGGUUGGAACA | [887-907] | [2439-2459] | [1023-1043] | |
| Single Sp | 21 | UGUGCACCGUAAUGAGAUGCU | AGCAUCUCAUUACGGUGCACA | [862-982] | [2414-2434] | [998-1018] | |
| Single Sp | 21 | UAUGUGCACCGUAAUGAGAUG | CAUCUCAUUACGGUGCACAUA | [860-880] | — | [2412-2432] | [996-1016] |
| Single Sp | 21 | UGGGUCUUCGCCUCUGUUAUG | CAUAACAGAGGCGAAGACCCA | [843-863] | — | [979-999] | |
| Single Sp | 21 | AGCCUGUGAAGUACGGACAGU | ACUGUCCGUACUUCACAGGCU | [820-840] | — | — | |
| Single Sp | 21 | AGGACUACUCCAAAGGGGUCA | GGACCCCUUUGGAGUAGUCCU | [739-759] | — | — | |
| Single Sp | 21 | AUGAUCAACAGCAACGAUGAC | GUCAUCGUUGCUGUUGAUCAU | [690-710] | [2242-2255] | [827-839] | |
| Single Sp | 21 | AAGAACCCGGCCAAAGACUGU | ACAGUCUUUGGCCGGGUUCUU | [624-644] | — | — | |
| Single Sp | 21 | ACUUAAAGAACCCGGCCAAAG | CUUUGGCCGGGUUCUUUAAGU | [616-638] | — | — | |
| Single Sp | 21 | UCACUUAAAGAACCCGGCCAA | UUGGCCGGGUUCUUUAAGUGA | [617-637] | — | — | |
| Single Sp | 21 | AUCACUUAAAGAACCCGGCCA | UGGCCGGGUUCUUUAAGUGAU | [616-636] | — | — | |
| Single Sp | 21 | CUGUAUCACUUAAAGAACCCG | CGGGUUCUUUAAGUGAUACAG | [612-632] | — | — | |
| Single Sp | 21 | AAGAGCCUGUAUCACUUAAAG | CUUUAAGUGAUACAGGCUCUU | [606-626] | — | [754-765] | |
| Single Sp | 21 | UGAGAUCCUGAACAAGAGCCU | AGGCUCUUGUUCAGGAUCUCA | [593-613] | [2145-2165] | [741-761] | |

### Example 2

### Testing the siRNA Compounds for Inhibition of TGase Activity

I. Preparation of Working Solutions of siRNAs (Double-Stranded Oligonucleotides)

Lyophilized oligonucleotides were dissolved in RNAse-free double-distilled water to produce a final concentration of 100 uM. The diluted oligonucleotides were kept at room temperature for 15 min and immediately frozen in liquid nitrogen.

The oligonucleotides were stored at −80° C. and diluted before use with PBS.

II. Activity Assay for siRNA Against TGase in vitro.

The enzymatic activity of TGase is measured. The activity of siRNA against TGase polypeptide is manifested by reduction in TGase enzymatic activity in transfected cells as compared to control cells.

III. Transfection by siRNA Oligonucleotides Using Lipofectamine2000 Reagent

2×10⁵ cells are seeded per well in 6 well-plates. After 24 hrs, the cells are transfected with TGase specific siRNA oligonucleotides with or without TGase 1 or TGase 3 or TGase 5 or TGase 7 expression plasmid using Lipofectamine2000 reagent (Invitrogen) according to the following procedure:
1. Before transfection, the cell medium is replaced with 1500 ul of fresh medium without antibiotics.
2. In a sterile plastic tube, Lipofectamine2000 reagent (the amount is calculated according to 5 ul per well) is added to 250 ul of serum-free medium, and incubated for 5 min at room temperature.
3. In another tube, the siRNA oligonucleotides. (varying amounts to fit the desired final concentration per well) with or without a tested expression plasmid are added to 250 ul of serum-free medium.
4. Lipofectamine2000 complex is combined with the siRNA solution and incubated for 20 min at room temperature.
5. The resulting mixture is added dropwise to the cells, and the cells are incubated at 37° C. until analysis of siRNA activity.

### Example 3

### Animal Model Systems of Kidney Fibrosis

Testing the active siRNA may be done in the following systems which have been studied as described below. The models are systems for testing the therapeutic efficacy of the inhibitors.

A. ZDF Rats

Samples of 9-month-old ZDF rats (Zucker diabetic fatty rats) presented hydronephrotic kidneys with dilated calyces. Microscopically these samples presented a picture of glomerulosclerosis and tubulointerstitial fibrosis. In accordance with these morphological changes, the expression of marker genes as measured by in situ hybridization (osteopontin (OPN), transforming growth factor β1 (TGF-β1) and procollagen α1(I) (Col1)) was significantly changed when compared to normal kidneys. Strong OPN expression was detectable in all tubular structures in both cortex and medulla. The TGF-β1 expression was widespread throughout interstitial cells. Some epithelial cells also showed TGF-β1 expression. Col1 expression was detectable by in situ hybridization in most interstitial cells within the medulla, while cortical expression was “focal”.

B. Aged fa/fa (Obese Zucker) Rats

Samples of 12-month-old fa/fa rats presented strong glomerulosclerosis and diffuse tubulointerstitial fibrosis throughout the cortex and the medulla. The pattern of marker gene expression corresponded to morphological changes. OPN was expressed by tubular structures in the cortex and the medulla. Multiple interstitial cells expressed TGF-β1. Significantly, multiple foci and single interstitial cells showed strong Col1 expression in both cortex and medulla so that the number of Col1-expressing cells appeared to be higher in fa/fa samples than in ZDF samples.

Interestingly, Col1 expression was not detected in glomeruli of either ZDF or fa/fa rats in spite of the prominent accumulation of collagen, as revealed by Sirius Red staining. This suggested a low steady state level of Col1 mRNA in glomerular cells.

C. Aged SD (Normal) Rats

Samples of aged SD rats showed increased accumulation of collagen in glomeruli and interstitial space and increased expression of the marker genes. Significantly, the intensity of fibrotic change varied among samples so that one of four samples studied displayed very few changes compared with young animals; fibrotic change in another sample was confined to “polar” regions, and two samples showed uniform accumulation of collagen and elevated expression of marker genes throughout the sections.

D. Goto Kakizaki (GK)/Wistar (Normal) 48-Week-Old Rats

Samples of both GK and Wistar 48-week-old rats showed an accumulation of collagen in glomeruli and interstitial space. This accumulation was more pronounced in the GK samples. Two samples were used for mRNA isolation: C9 and GK9. Both were hybridized to the probe specific for IGFBP4. The in situ hybridization results showed that the GK sample demonstrated elevated expression of this gene.

E. Permanent UUO

Another known animal model in which mainly kidney fibrosis is evident, but without a background of diabetes, is unilateral ureteral obstruction (UUO) in which interstitial fibrosis is rapid and occurs within days following the obstruction.

A known model for fibrosis was employed—unilateral urether occlusion (UUO). One of the urethers was occluded (see below) and animals were sacrificed 1,5,10,15,20 and 25 days following occlusion.

Permanent UUO resulted in rapid activation (5 days of UUO) of collagen synthesis by interstitial cells in both medulla and cortex. By 20-25 days of UUO, significant amounts of interstitial collagen were deposited in the interstitial space while glomerular accumulation of collagen was confined to the outer capsule. Thus, permanent UUO samples provided an acute model of tubulointerstitial renal fibrosis without prominent glomerulosclerotic changes.

F. 5/6 Nephrectomy

5/6 nephrectomy is another useful animal model for chronic renal insufficiency (CRI) in which fibrosis is evident.

1. A compound having the structure:
5′ (N)ₓ—Z 3′ antisense strand
3′ Z′—(N′)_{y}5′ sense strand
wherein each N and N′ is a ribonucleotide which may be modified or unmodified in its sugar residue and (N)ₓ and (N′)y is an oligomer in which each consecutive N or N′ is joined to the next N or N′ by covalent bond;
wherein each of x and y is an integer between 17 and 40;
wherein each of Z and Z′ may be present or absent, but if present is dTdT and is covalently attached at the 3′ terminus;
and wherein the sequence of (N)ₓ comprises any one of the antisense sequences present in Table A.

2. The compound of claim 1, wherein the covalent bond is a phosphodiester bond.

3. The compound of claim 2, wherein x=y.

4. The compound of claim 2, wherein x=y=19.

5. The compound of claim 1, wherein Z and Z′ are both absent.

6. The compound of claim 1, wherein Z or Z′ is present.

7. The compound of claim 1, wherein all of the ribonucleotides are unmodified in their sugar residues.

8. The compound of claim 1, wherein at least one ribonucleotide is modified in its sugar residue.

9. The compound of claim 8, wherein the modification of the sugar residue comprises a modification at the 2′ position.

10. The compound of claim 9, wherein the modification at the 2′ position is selected from the group comprising amino, fluoro, methoxy, alkoxy and allcyl.

11. The compound of claim 10, wherein the modification at the 2′ position is methoxy (2′-0-methyl).

12. The compound of claim 1, wherein alternating ribonucleotides are modified in both the antisense and the sense strands.

13. The compound of claim 1 wherein the ribonucleotides at the 5′ and 3′ termini of the antisense strand are modified in their sugar residues, and the ribonucleotides at the 5′ and 3′ termini of the sense strand are unmodified in their sugar residues.

14. The compound of claim 1 wherein the antisense strand is phophorylated at the 5′terminus, and may or may not be phophorylated at the 3′terminus; and wherein the sense strand may or may not be phophorylated at the 5′terminus and at the 3′terminus

15. A vector capable of expressing the compound of claim 1.

16. A composition comprising the compound of claim 1 and a carrier.

17. A composition of claim 16, wherein the carrier comprises a pharmaceutically acceptable carrier.

18. A composition comprising a carrier and the compound of claim 1 in an amount effective to down-regulate expression in a cell of a TGase gene which comprises a sequence substantially complementary to the sequence of (N)ₓ.

19. (canceled)

20. A method of down-regulating the expression of a gene of the TGase family by at least 50% as compared to a control comprising contacting an mRNA transcript of the gene with a compound of claim 1.

21. A method of treating a patient suffering from fibrosis-related pathology comprising administering to the patient a composition of claim 16 in a therapeutically effective dose so as to thereby treat the patient.

22. Use of a therapeutically effective amount of a compound of claim 1 for the preparation of a composition for promoting recovery in a patient suffering from kidney or liver fibrosis, ocular scarring, cataract or glaucoma.

23. A method of treating a patient suffering from a fibrosis-related pathology comprising administering to the patient a composition comprising an inhibitor of the TGase family in a therapeutically effective dose so as to thereby treat the patient.

24. A method of claim 23, wherein the inhibitor is an siRNA.

25. A method of claim 23, wherein the inhibitor is an antibody

26. A method of claim 23, wherein the TGase is TGase 1, TGase 3, TGase 5 or TGase7.

27. A method of claim 26, wherein the inhibitor is an siRNA which specifically inhibits expression of TGase.

28. A method of claim 23, wherein the inhibitor is an antibody which specifically inhibits the activity of a TGase polypeptide.


SEQUENCE LISTING (30 sequences)

SEQ ID NO: 1 - DNA, 2064 bp, Homo sapiens
  CDS (1)..(2064)
atggccgaggagctggtcttagagaggtgtgatctggagctggagaccaatggccgagaccaccacacggccgacctgtgccgggagaagctggtggtgcgacggggccagcccttctggctgaccctgcactttgagggccgcaactacgaggccagtgtagacagtctcaccttcagtgtcgtgaccggcccagcccctagccaggaggccgggaccaaggcccgttttccactaagagatgctgtggaggagggtgactggacagccaccgtggtggaccagcaagactgcaccctctcgctgcagctcaccaccccggccaacgcccccatcggcctgtatcgcctcagcctggaggcctccactggctaccagggatccagctttgtgctgggccacttcattttgctcttcaacgcctggtgcccagcggatgctgtgtacctggactcggaagaggagcggcaggagtatgtcctcacccagcagggctttatctaccagggctcggccaagttcatcaagaacataccttggaattttgggcagtttgaagatgggatcctagacatctgcctgatccttctagatgtcaaccccaagttcctgaagaacgccggccgtgactgctcccgccgcagcagccccgtctacgtgggccgggtggtgagtggcatggtcaactgcaacgatgaccagggtgtgctgctgggacgctgggacaacaactacggggacggcgtcagccccatgtcctggatcggcagcgtggacatcctgcggcgctggaagaaccacggctgccagcgcgtcaagtatggccagtgctgggtcttcgccgccgtggcctgcacagtgctgaggtgcctgggcatccctacccgcgtcgtgaccaactacaactcggcccatgaccagaacagcaaccttctcatcgagtacttccgcaatgagtttggggagatccagggtgacaagagcgagatgatctggaacttccactgctgggtggagtcgtggatgaccaggccggacctgcagccggggtacgagggctggcaggccctggacccaacgccccaggagaagagcgaagggacgtactgctgtggcccagttccagttcgtgccatcaaggagggcgacctgagcaccaagtacgatgcgccctttgtctttgcggaggtcaatgccgacgtggtagactggatccagcaggacgatgggtctgtgcacaaatccatcaaccgttccctgatcgttgggctgaagatcagcactaagagcgtgggccgagacgagcgggaggatatcacccacacctacaaatacccagaggggtcctcagaggagagggaggccttcacaagggcgaaccacctgaacaaactggccgagaaggaggagacagggatggccatgcggatccgtgtgggccagagcatgaacatgggcagtgactttgacgtctttgcccacatcaccaacaacaccgctgaggagtacgtctgccgcctcctgctctgtgcccgcaccgtcagctacaatgggatcttggggcccgagtgtggcaccaagtacctgctcaacctcaacctggagcctttctctgagaagagcgttcctctttgcatcctctatgagaaataccgtgactgccttacggagtccaacctcatcaaggtgcgggccctcctcgtggagccagttatcaacagctacctgctggctgagagggacctctacctggagaatccagaaatcaagatccggatccttggggagcccaagcagaaacgcaagctggtggctgaggtgtccctgcagaacccgctccctgtggccctggaaggctgcaccttcactgtggagggggccggcctgactgaggagcagaagacggtggagatcccagaccccgtggaggcaggggaggaagttaaggtgagaatggacctgctgccgctccacatgggcctccacaagctggtggtgaacttcgagagcgacaagctgaaggctgtgaagggcttccggaatgtcatcattggccccgcctaa

SEQ ID NO: 2 - protein, 687 aa, Homo sapiens
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

SEQ ID NO: 3 - RNA, 19 nt, artificial
  RNA Oligomer
gcaggaguauguucuuaau

SEQ ID NO: 4 - RNA, 19 nt, artificial
  RNA Oligomer
gcagcaaacccaaugugua

SEQ ID NO: 5 - RNA, 19 nt, artificial
  RNA Oligomer
cucccuggaugacaaugga

SEQ ID NO: 6 - RNA, 19 nt, artificial
  RNA Oligomer
gaacucccuggaugacaau

SEQ ID NO: 7 - RNA, 19 nt, artificial
  RNA Oligomer
ggagauccugcuuagcuac

SEQ ID NO: 8 - RNA, 19 nt, artificial
  RNA Oligomer
uggagauccugcuuagcua

SEQ ID NO: 9 - RNA, 19 nt, artificial
  RNA Oligomer
guggagauccugcuuagcu

SEQ ID NO: 10 - RNA, 19 nt, artificial
  RNA Oligomer
ggcagcaaacccaaugugu

SEQ ID NO: 11 - RNA, 19 nt, artificial
  RNA Oligomer
ccaugcugcucaaugucuc

SEQ ID NO: 12 - RNA, 19 nt, artificial
  RNA Oligomer
gugacaagguguacuggca

SEQ ID NO: 13 - RNA, 19 nt, artificial
  RNA Oligomer
ggugaauagugacaaggug

SEQ ID NO: 14 - RNA, 19 nt, artificial
  RNA Oligomer
ccauggacaucuacuuuga

SEQ ID NO: 15 - RNA, 19 nt, artificial
  RNA Oligomer
cuggaacuauggccaguuu

SEQ ID NO: 16 - RNA, 19 nt, artificial
  RNA Oligomer
ccaucauuggcaaauucca

SEQ ID NO: 17 - RNA, 19 nt, artificial
  RNA Oligomer
auuaagaacauacuccugc

SEQ ID NO: 18 - RNA, 19 nt, artificial
  RNA Oligomer
uacacauuggguuugcugc

SEQ ID NO: 19 - RNA, 19 nt, artificial
  RNA Oligomer
uccauugucauccagggag

SEQ ID NO: 20 - RNA, 19 nt, artificial
  RNA Oligomer
auugucauccagggaguuc

SEQ ID NO: 21 - RNA, 19 nt, artificial
  RNA Oligomer
guagcuaagcaggaucucc

SEQ ID NO: 22 - RNA, 19 nt, artificial
  RNA Oligomer
uagcuaagcaggaucucca

SEQ ID NO: 23 - RNA, 19 nt, artificial
  RNA Oligomer
agcuaagcaggaucuccac

SEQ ID NO: 24 - RNA, 19 nt, artificial
  RNA Oligomer
acacauuggguuugcugcc

SEQ ID NO: 25 - RNA, 19 nt, artificial
  RNA Oligomer
gagacauugagcagcaugg

SEQ ID NO: 26 - RNA, 19 nt, artificial
  RNA Oligomer
ugccaguacaccuugucac

SEQ ID NO: 27 - RNA, 19 nt, artificial
  RNA Oligomer
caccuugucacuauucacc

SEQ ID NO: 28 - RNA, 19 nt, artificial
  RNA Oligomer
ucaaaguagauguccaugg

SEQ ID NO: 29 - RNA, 19 nt, artificial
  RNA Oligomer
aaacuggccauaguuccag

SEQ ID NO: 30 - RNA, 19 nt, artificial
  RNA Oligomer
uggaauuugccaaugaugg